(19) United States
(12) Patent Application Publication
(10) Pub. No.: US 2021/0155664 A1
(43) Pub. Date: May 27, 2021

(54) REGENERATING FUNCTIONAL NEURONS FOR TREATMENT OF NEUROLOGICAL DISORDERS

(71) Applicant: The Penn State Research Foundation, University Park, PA (US)

(72) Inventor: Gong Chen, State College, PA (US)

(21) Appl. No.: 17/072,946

(22) Filed: Oct. 16, 2020

Related U.S. Application Data
(60) Provisional application No. 62/916,702, filed on Oct. 17, 2019.

(51) Int. Cl. C07K 14/47; C12N 15/86; A61P 25/28

(52) U.S. Cl. CPC C07K 14/4705; C12N 2750/14141; A61P 25/28; C12N 15/86

(57) ABSTRACT

This document provides methods and materials involved in treating mammals having a neurological disorder in the brain (e.g., Alzheimer's disease). For example, methods and materials for administering a composition including exogenous nucleic acid encoding a NeuroD1 polypeptide to a mammal having a neurological disorder in the brain are provided.

### CROSS REFERENCE TO RELATED APPLICATIONS

This application claims priority to U.S. Application Ser. No. 62/916,702, filed on Oct. 17, 2019, the contents of this aforementioned application are fully incorporated herein by reference.

### GOVERNMENT SUPPORT

This invention was made with government support under Grant No. AG045656 awarded by the National Institutes of Health. The Government has certain rights in the invention.

### BACKGROUND

### 1. Technical Field

This document relates to methods and materials involved in treating mammals having a neurological disorder in the brain (e.g., Alzheimer's disease). For example, this document provides methods and materials for administering a composition containing exogenous nucleic acid encoding a NeuroD1 polypeptide (or a biologically active fragment thereof) to a mammal having a neurological disorder in the brain.

### 2. Background Information

Neurological disorders, including Alzheimer's disease (AD), are characterized by cognitive dysfunction and memory deficits (Querfurth and Laferla, New Engl. J. Med., 362:329-344 (2010)). Studies on postmortem tissue discovered that the brains of AD patients were both lighter and physically smaller compared to a healthy brain, indicating severe neuron and tissue loss in AD progression (Gomez-Isla et al., J. Neuroscience, 16:4491-4500 (1996)). Astrocytes play a role in transporting neurotrophic factors and metabolic cytokines, maintaining the interactions between neurons and other types of cells and supporting the neuronal circuits (Burda and Sofroniew, Neuron, 81:229-248 (2014)). Disruption of these functions promote the neuroinflammation and leads to a more severe AD pathological progression (Qin and Benveniste, Methods Mol. Biol., 814:235-249 (2012)). The down-stream effects of this gradual and progressive disturbance at the cellular and molecular level are the impairment of the learning and memory systems and behavioral abnormalities (Huang and Mucke, Cell, 148:1204-1222 (2012); and Kunz et al., Science, 350:430-433 (2015)). Astrocytes are star-shaped glial cells distributed in the brain and spinal cord. In the normal brain, astrocytes maintain the resting state with normal morphology and minimal GFAP immunoreactivity. Microglia are the brain-resident macrophages distributed ubiquitously in the brain. They are developmentally distinct from other tissue-resident macrophage populations (Ginhoux et al., Science, 330:841-845 (2010); and Sheng et al., Immunity, 43:382-393 (2015)). There is emerging evidence highlighting the importance of microglia's role in the AD pathology. In fact, a novel insight suggested that the microglia can form and secrete apoptosis-associated speck-like protein containing C-terminal caspase recruitment domain (ASC) specks once it detects the extracellular stimulus; such ASC specks facilitate the seeding and progression of amyloid deposition in the brain of AD (Venegas et al., Nature, 552:355-361 (2017)). There are currently no effective therapeutic approaches for those suffering from AD.

### SUMMARY

This document relates to methods and materials involved in treating mammals having a neurological disorder (e.g., Alzheimer's disease). For example, this document provides methods and materials for administering a composition containing exogenous nucleic acid encoding a NeuroD11 polypeptide (or a biologically active fragment thereof) to a mammal having a neurological disorder in the brain.

In general, one aspect of this document features a method for treating a mammal having a neurological disorder in the brain. The method comprises (or consists essentially of or consists of) administering a composition comprising exogenous nucleic acid encoding a Neurogenic Differentiation 1 (NeuroD1) polypeptide or a biologically active fragment thereof to the brain of the mammal. The mammal can be a human. The neurological disorder can be Alzheimer's disease. The administering step can comprise delivering an expression vector comprising a nucleic acid encoding NeuroD1 to the brain. The administering step can comprise delivering a recombinant viral expression vector comprising a nucleic acid encoding NeuroD1 to the brain. The administering step can comprise delivering a recombinant adeno-associated virus expression vector comprising a nucleic acid encoding NeuroD1 to the brain. The adeno-associated virus can be an AAV.PHP.eB. The administering step can comprise administering a recombinant expression vector comprising a nucleic acid sequence encoding NeuroD1 protein, wherein the nucleic acid sequence encoding NeuroD1 protein comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO:2 or SEQ ID NO:4, or a functional fragment thereof. The administering step can comprise a stereotactic intracranial injection. The administering step can comprise two or more stereotactic intracranial injections. The administering step can comprise an extracranial injection. The administering step can comprise two or more extracranial injections. The administering step can comprise a retro-orbital injection.

In another aspect, this document features a method of treating a mammal having Alzheimer's disease. The method comprises (or consists essentially of or consists of) administering a pharmaceutical composition comprising a pharmaceutically acceptable carrier containing adeno-associated virus particles comprising a nucleic acid encoding a NeuroD1 polypeptide or a biologically active fragment thereof to the brain of the mammal. The pharmaceutical composition can comprise about 1 μL to about 500 μL of a pharmaceutically acceptable carrier containing adeno-associated virus particles at a concentration of 10¹⁰-10¹⁴ adeno-associated virus particles/mL of carrier. The pharmaceutical composition can be injected in the brain of the mammal at a controlled flow rate of about 0.1 μL/minute to about 5 μL/minute.

In another aspect, this document features a method for (1) reducing neurofibrillary tangles of hyperphosphorylated tau protein, (2) reducing aggregation of extracellular amyloid plaques, (3) reducing neuroinflammation, (4) reducing interleukin 1β (IL-1β), (5) generating new glutamatergic neurons, (6) increasing survival of GABAergic neurons, (7) generating new non-reactive astrocytes, (8) reducing the number of reactive astrocytes, or (9) improving memory within a mammal having Alzheimer's disease and in need of the (1), (2), (3), (4), (5), (6), (7), (8) or (9). The method comprises (or consists essentially of or consists of) administering a composition comprising exogenous nucleic acid encoding a NeuroD1 polypeptide or a biologically active fragment thereof to the mammal, wherein the (1) hyperphosphorylated neurofibrillary tau protein tangles are reduced, (2) aggregation of extracellular amyloid plaques is reduced, (3) neuroinflammation is reduced, (4) interleukin 1β (IL-1β) levels are reduced, (5) new glutamatergic neurons are generated, (6) survival of GABAergic neurons is increased, (7) new non-reactive astrocytes are generated, (8) the number of reactive astrocytes is reduced, or (9) the memory is improved. The mammal can be a human. The administering step can comprise delivering an expression vector comprising a nucleic acid encoding a NeuroD1 polypeptide. The administering step can comprise delivering a recombinant viral expression vector comprising a nucleic acid encoding a NeuroD1 polypeptide. The administering step can comprise delivering a recombinant adeno-associated virus expression vector comprising a nucleic acid encoding a NeuroD1 polypeptide. The recombinant adeno-associated virus expression vector can be an AAV.PHP.eB expression vector. The administering step can comprise administering a recombinant expression vector comprising a nucleic acid sequence encoding a NeuroD1 polypeptide, wherein the nucleic acid sequence encoding a NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO:2 or SEQ ID NO:4, or a functional fragment thereof. The administering step can comprise a stereotactic intracranial injection. The administering step can comprise two or more stereotactic intracranial injections. The administering step can comprise an extracranial injection. The administering step can comprise two or more extracranial injections. The administering step can comprise a retro-orbital injection.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1H. NeuroD1 over-expression enables in vivo reprogramming of reactive astrocytes into functional neurons in 5×FAD cortex. (FIG. 1A) Schematic illustration of constructing the AAV9 vector expressing the target gene under 10 cre-loxP regulation system in reactive astrocytes. (FIG. 1B) Stereotactic injection inside the mouse cortex enabled accurate delivery of target genes in vivo. (FIG. 1C) NeuroD1 over-expression achieved astrocyte-to-neuron conversion in cortex with high efficiency by 30 days post-injection (DPI). The right panel showed the enlarged images of the circled region by the dotted line on the left panel. Note that in control group where only have GFP over-expression in reactive astrocytes still remain typical glial cell morphology; NeuroD1 over-expression cells already possess neuronal-like morphology. Scale bar=30 μm. (FIG. 1D) At 30 days after injection, AAV9-GFAP-Cre and AAV9-CAG-GFP infected cells (which, when viewed in color, stained green) were immunopositive for reactive astrocyte marker GFAP (which, when viewed in color, stained magenta). AAV9-GFAP-Cre and AAV9-CAG-NeuroD1-P2A-GFP infected cells (which, when viewed in color, stained green) were immunopositive for NeuroD1 (which, when viewed in color, stained red), but immunonegative for reactive astrocyte marker GFAP. Scale bar=30 μm. (FIG. 1E) At 30 days after injection, AAV9-GFAP-Cre and AAV9-CAG-GFP infected cells (which, when viewed in color, stained green) were immunonegative for mature neuronal marker NeuN (which, when viewed in color, stained magenta). AAV9-GFAP-Cre and AAV9-CAG-NeuroD1-P2A-GFP infected cells (which, when viewed in color, stained green) were immunopositive for mature neuronal marker NeuN, and these cells had high expression level of NeuroD1 (which, when viewed in color, stained red). Scale bar=30 μm. (FIG. 1F) A typical phase contrast image showing the whole cell recording of the converted neuron in the cortex region of 5×FAD mouse brain (26 DPI). Scale bar=10 (FIG. 1G) Representative trace from cortical slice recordings showing repetitive action potentials in NeuroD1-converted neurons (26 DPI). (FIG. 1H) Representative traces showing spontaneous synaptic events in a NeuroD1-converted neuron (26 DPI) in cortical slice recording. A representative trace of sEPSC and sIPSC was enlarged at the bottom of this figure panel.

FIGS. 2A-2F. NeuroD1-mediated astrocyte-to-neuron conversion ameliorates the hyperactive reactive astrocytes in 5×FAD mouse cortex. (FIG. 2A) The 5×FAD mice were treated by either GFP or NeuroD1 via AAV9 virus delivery system at 4 months old and were further dissected for immunostaining and analysis at 60 DPI. Representative images showing the reactive astrocytes were reduced after NeuroD1-mediated conversion in 5×FAD mouse cortex. Note that in GFP control, abundant reactive astrocytes (which appeared white) existed in both of the injection core and surrounding regions. However, the reactive astrocytes (which appeared white) were reduced in the injection core of NeuroD1 group, accompanied with a large number of neuronal morphology cells (which appeared yellow) regenerated in situ. (FIG. 2B) Enlarged representative images showing the differences (morphology and number) between the reactive astrocytes (GFAP, which, when viewed in color, stained magenta) in GFP control group and NeuroD1-mediated cell conversion group. Scale bar represent 30 (FIG. 2C) Co-labeling of reactive astrocytes marker GFAP (which, when viewed in color, stained red) and amyloid plaques (which, when viewed in color, stained blue, Thioflavin-S dye) in 5×FAD cortex. Scale bars represent 30 N=8 GFP-treated 5×FAD mice, and 8 NeuroD1-treated 5×FAD mice. Data are presented as mean±s.e.m., * p<0.05; ** p<0.01; *** p<0.001; Student's t-test. (FIG. 2D) Quantitative analysis on the reactive astrocytes number. (FIG. 2E) Quantitative analysis on the reactive astrocytes covered region percentage. (FIG. 2F) Quantitative analysis on the reactive astrocytes marker GFAP intensity.

FIGS. 3A-3D. NeuroD1-induced astrocyte-to-neuron conversion can recover the neuron loss in the 5×FAD mouse cortex. (FIG. 3A) The 5×FAD mice were treated by either GFP or NeuroD1 via AAV9 virus delivery system at 4 months old and were further dissected for immunostaining and analysis at 60 DPI. The representative images showing the neuron density (NeuN, which, when viewed in color, stained red) was increased in NeuroD1 conversion group at 60 days post-injection. (FIG. 3B) Enlarged images showing the increased number of mature neurons (NeuN, which, when viewed in color, stained red) in NeuroD1 group. Note that GFP+ (which, when viewed in color, stained green) and NeuN+ (which, when viewed in color, stained red) cells were newly-converted neurons (which merged as yellow) by NeuroD1. Scale bar represents 30 N=6 GFP-treated 5×FAD mice and 6 NeuroD1-treated 5×FAD mice. Data are presented as mean±s.e.m., * p<0.05; ** p<0.01; *** p<0.001; Student's t-test. (FIG. 3C) Quantitative results indicated a significantly increase of mature neurons (NeuN+ cell) in NeuroD1 group. (FIG. 3D) Quantification of neuron and astrocytes ratio.

FIGS. 4A-4C. GABAergic neurons can be regenerated via NeuroD1-mediated astrocyte-to-neuron conversion in 5×FAD mouse cortex. (FIG. 4A) The 5×FAD mice were treated by either GFP or NeuroD1 via AAV9 virus delivery system at 4 months old and were further dissected for analysis at 60 DPI. Representative images showing the distribution of GABAergic neurons (GABA+ and NeuN+ cell) and all converted neurons (GFP+ and NeuN+ cell) in NeuroD1 treated 5×FAD mouse cortex. Note that the GFP+, NeuN+, and GABA+ triple immunopositive cells are converted GABAergic neurons. (FIG. 4B) Enlarged images showing the existing GABA+ neuron (arrow, GABA+ and NeuN+ cell) and converted GABA+ neuron (arrow head, GFP+, GABA+, and NeuN+ cell) in 5×FAD cortex. Scale bar represents 30 N=8 NeuroD1 treated 5×FAD mouse (60 DPI). (FIG. 4C) Enlarged images showing the existing GAD67+ neuron (arrow, GAD67+ and NeuN+ cell) and converted GAD67+ neuron (arrow head, GFP+, GABA+, and NeuN+ cell) in 5×FAD cortex. Scale bar represents 30 N=8 NeuroD1 treated 5×FAD mouse (60 DPI).

FIG. 5. NeuroD1 group has less abnormal GFP aggregates in 5×FAD mouse cortex. The 5×FAD mice were treated by either GFP or NeuroD1 via AAV9 virus delivery system at 4 months old and were further dissected for analysis at 60 DPI. GFP aggregates were observed majorly in the treated brain region in GFP control group (the top row); this phenomenon is much reduced in NeuroD1 treated group (bottom row). Scale bar represents 30 μm.

FIGS. 6A-E. NeuroD1-mediated astrocyte-to-neuron conversion mitigated the intracellular Aβ level in 5×FAD cortical neurons. (FIG. 6A) The 5×FAD mice were treated by either GFP or NeuroD1 via AAV9 virus delivery system at 4 months old and were further dissected for immunostaining and analysis at 60 DPI. Representative confocal micrographs showing the Aβ level (Aβ42, which, when viewed in color, stained sapphire) in 5×FAD mouse brain cortex region. Intracellular Aβ42 level (Aβ42 intensity in NeuN+ regions) of all neurons from the infection core of brain samples were carefully measured and quantified. Arrow: pre-existing neurons; arrowhead: converted neurons. Scale bar represents 30 μm. (FIG. 6B) Quantitative results of neuron number in the infection core of GFP control group and NeuroD1 group. N=3 GFP-treated 5×FAD mice and 3 NeuroD1-treated 5×FAD mice. Data are presented as mean±s.e.m., * p<0.05; ** p<0.01; *** p<0.001; Student's t-test. (FIG. 6C) Quantitative results of pre-existing neuron number in the infection core of GFP control group, pre-existing neuron number in the infection core of NeuroD1 group and converted neuron number in the infection core of NeuroD1 group, respectively. Note that no difference was observed between the pre-existing neuron number in GFP control group and NeuroD1 group. N=3 GFP-treated 5×FAD mice and 3 NeuroD1-treated 5×FAD mice. Data are presented as mean s.e.m., * p<0.05; ** p<0.01; *** p<0.001; one-way ANOVA with the Tukey's post-hoc test when comparing to multiple groups. (FIG. 6D) Quantitative results of intensity of intracellular Aβ42 in all neurons inside the infection core in GFP group and NeuroD1 group. Neuron number=662 from the infection core of three 5×FAD mice treated with GFP control; neuron number=1357 from the infection core of three 5×FAD mice treated with NeuroD1. Data are presented as mean±s.e.m., * p<0.05; ** p<0.01; *** p<0.001; Student's t-test. (FIG. 6E) Quantitative data showing the intensity of intracellular Aβ42 in all neurons in the infection core in GFP group and NeuroD1 group. Black bar: pre-existing neuron number=662 from the infection core of three 5×FAD mice in the GFP control group; white bar: pre-existing neuron number=714 from the infection core of three 5×FAD mice in the NeuroD1 group; and grey bar: converted neuron number=643 from the infection core of three 5×FAD mice in the NeuroD1 group. Data are presented as mean±s.e.m., * p<0.05; ** p<0.01; *** p<0.001; one-way ANOVA with the Tukey's post-hoc test when comparing to multiple groups.

FIGS. 7A-7D. NeuroD1-mediated astrocyte-to-neuron conversion enables the mitigation of pro-inflammatory microglia. (FIG. 7A) The 5×FAD mice were treated by either GFP or NeuroD1 via AAV9 virus delivery system at 4 months old and were further dissected for immunostaining and analysis at 60 DPI. Representative confocal images showing the changes of general microglia (Iba1, which, when viewed in color, stained grey) and the pro-inflammatory microglia subtype (iNOS, which, when viewed in color, stained red). Scale bar represents 30 μm. (FIG. 7B) Quantification data showing no significant differences of the Iba1 intensity between the GFP control group and NeuroD1 group. N=8 5×FAD mice in GFP group and 8 5×FAD mice in NeuroD1 group. Data are presented as mean±s.e.m., Student's t-test. (FIG. 7C) Quantitative results showing no significant difference of Iba1+ microglia covered area percentage between GFP and NeuroD1 group. N=8 5×FAD mice in GFP group and 8 5×FAD mice in NeuroD1 group. Data are presented as mean±s.e.m., Student's t-test. (FIG. 7D) The intensity of pro-inflammatory microglia subtype (iNOS+ microglia) is largely mitigated in NeuroD1 group. N=8 5×FAD mice in GFP group and 8 5×FAD mice in NeuroD1 group. Data are presented as mean±s.e.m., * p<0.05; ** p<0.01; *** p<0.001; Student's t-test.

FIGS. 8A-8B. Pro-inflammatory cytokine IL-10 reduced after NeuroD1-mediated cell conversion in 5×FAD cortex. (FIG. 8A) The 5×FAD mice were treated by either GFP or NeuroD1 via AAV9 virus delivery system at 4 months old and were further dissected for immunostaining and analysis at 60 DPI. Representative confocal images showing the reduction of reactive astrocytes (which, when viewed in color, stained magenta) and interleukin-1β (which, when viewed in color, stained red) after NeuroD1 treatment. Scale bar represents 30 μm. (FIG. 8B) Quantification analysis showing a significantly decrease of intensity of IL-1β after NeuroD1-induced cell conversion. N=8 5×FAD mice in GFP group and 8 5×FAD mice in NeuroD1 group. Data are presented as mean±s.e.m., * p<0.05; ** p<0.01; *** p<0.001; Student's t-test.

FIGS. 9A-9D. NeuroD1-converted neurons can survive for more than 8 months with good morphology in 5×FAD brain. (FIG. 9A) The 5×FAD mice were treated by either GFP or NeuroD1 via AAV9 virus delivery system at 6 months old and were further dissected for immunostaining and analysis at 8 months post-injection. Representative images showing the infected cells in GFP control group and NeuroD1 group. Note that in NeuroD1 group, converted neurons (GFP+, which, when viewed in color, stained green) were distributed evenly throughout the cortex region of 5×FAD mice. (FIG. 9B to FIG. 9D) Typical micrographs showing the astrocytes, converted neurons, and NeuroD1 expression level in the infection core in the cortex region. In GFP control group, astrocytes (GFAP+, which, when viewed in color, stained magenta) were still hyper-active, and abundant abnormal GFP aggregates (which, when viewed in color, stained green) were observed. In NeuroD1 group, infected astrocytes have been successfully converted to mature neurons (NeuN+, which, when viewed in color, stained red) with high expression level of NeuroD1 and healthy morphology with strong neurites (which, when viewed in color, stained green). Scale bar represents 30 μm.

FIGS. 10A-10F. Axons, dendrites, and synapses increase after NeuroD1-mediated cell conversion in 5×FAD cortex after long-term. (FIG. 10A) The 5×FAD mice were treated by either GFP or NeuroD1 via AAV9 virus delivery system at 6 months old and were further dissected for immunostaining and analysis at 8 months post-injection. Representative images revealed the increase of axons (NF200, which, when viewed in color, stained sapphire) and synapses (Synaptophysin, which, when viewed in color, stained red) after NeuroD1-induced conversion in the infected cortex. Scale bar represents 30 N=3. Data are presented as mean±s.e.m., * p<0.05; ** p<0.01; *** p<0.001; Student's t-test. (FIG. 10B) Quantitative analyses of intensity of axon marker NF200. (FIG. 10C) Quantitative analyses of intensity of synapse marker synaptophysin. (FIG. 10D) Representative images revealed the increase of dendrites (MAP2, which, when viewed in color, stained sapphire) and excitatory synapses (vGlut1, which, when viewed in color, stained red) after NeuroD1 treatment at 8 months after injection (mpi). Scale bar represents 30 (FIG. 10E) Quantitative analyses of intensity of dendrite marker MAP2. (FIG. 10F) Quantitative analyses of intensity of excitatory synaptic marker vGlut1.

FIGS. 11A-11D. NeuroD1-mediated astrocyte-to-neuron conversion protects the blood vessel integrity in 5×FAD brain cortex. (FIG. 11A) Typical images showing the blood vessel segments (Ly6C, which, when viewed in color, stained red) and astrocytes endfeet (AQP4, which, when viewed in color, stained magenta) on blood vessels in 5×FAD brain. The 5×FAD mice were treated by either GFP or NeuroD1 via AAV9 virus delivery system at 6 months old and were further dissected for immunostaining and analysis at 8 months post-injection. (FIG. 11B) Representative images of blood vessel segment (Ly6C, which, when viewed in color, stained red) and astrocytes endfeet (AQP4, which, when viewed in color, stained sapphire) in 5×FAD mice cortex at 8 months after NeuroD1 or GFP intervention. Scale bar represents 30 (FIG. 11C) Quantitative analyses of the length of AQP4+ segments. N=three intact WT mice, three 5×FAD mice in GFP control group, three 5×FAD mice in NeuroD1 group, age and gender matched. Data are presented as mean±s.e.m., * p<0.05; ** p<0.01; *** p<0.001; Student's t-test. (FIG. 11D) Quantitative analyses of the length of Ly6C+ blood vessel segments.

FIGS. 12A-12H. Multiple intracranial microinjections for global infection. (FIG. 12A to FIG. 12B) Map of the AAV Cre-FLEX system used to activate GFP or NeuroD1 express. (FIG. 12C to FIG. 12D) Schematic diagram showing the multiple intracranial injection sites in the mouse brain. The four injection windows were labeled by the four dots on the mouse skull (FIG. 12C), and the injection sites were indicated by the arrows and crosses (FIG. 12D). (FIG. 12E to FIG. 12F) The sagittal and coronal views of the GFAP::Cre transgenic mouse brain 15 DPI of GFP virus injection. (FIG. 12G to FIG. 12H) The sagittal and coronal views of the GFAP::Cre transgenic mouse brain 15 DPI of NeuroD1-P2A-GFP virus injection. Astrocytes were labeled by S1000 (which, when viewed in color, stained red) and neurons were labeled by NeuN (which, when viewed in color, stained cyan). The dash line 1 and 2 in (FIG. 12F) and (FIG. 12H) indicate the relative coronal section position showing in the bottom panels.

FIGS. 13A-13B. NeuroD1 mediated global conversion in the GFAP::Cre transgenic mouse brain. (FIG. 13A) The overview of the GFP control virus injection (15 DPI) in the GFAP::Cre transgenic mouse brain, the high mag images of the different area were shown in the number 1 to 9 panels. Note that almost all of the GFP positive cells were co-labeled with astrocytic marker S1000 (which, when viewed in color, stained red). (FIG. 13B) The overview of the NeuroD1-P2A-GFP virus injection (15 DPI) in the GFAP::Cre transgenic mouse brain, the high mag images of the different area were shown in the number 1 to 9 panels. Note that the majority of the GFP positive cells in the cortical area (1, 3), hippocampus (4-6), subiculum (7), and middle brain (9) were co-labeled with neuronal marker NeuN (which, when viewed in color, stained cyan). The relative regions of number 1 to 9 panels are indicated in the low mag sagittal images.

FIGS. 14A-14B. Direct comparison of the morphologic differences between in the control and neuroD1 treated mouse. (FIG. 14A) The GFP positive cells in control and NeuroD1 treated mouse in the different areas. 15 DPI, almost all of the GFP positive cells in control groups still showing the typical astrocytic morphology. However, the NeuroD1 treated mouse the majority of the GFP positive cells have shown the typical neuronal morphology. (FIG. 14B) The strong NeuroD1 expression (which, when viewed in color, stained red, arrows) were revealed by immunostaining in the GFP positive cells in the ND1-P2A-GFP treated mouse brain and the NeuroD1 positive cells were also co-localized with NeuN (which, when viewed in color, stained cyan, arrows). In the control group, none of the GFP positive cells contained NeuroD1 or co-localized with NeuN (top).

FIGS. 15A-15C. Global astrocytes-to-neurons conversion in the 5×FAD mouse brain. (FIG. 15A) The work flow of the study. (FIG. 15B) Broad infection area was observed in the 5×FAD mouse brain. The Aβ plaques were revealed by the thioflavin-S staining (which, when viewed in color, stained blue). (FIG. 15C) Almost all of the GFP positive cells were co-labeled with the neuronal marker NeuN (which, when viewed in color, stained red) in the different brain area.

FIGS. 16A-16B. Characterization of the NeuroD1 converted neurons in the 5×FAD cerebral cortex. (FIG. 16A) The sagittal view of the NeuroD1 treated 5×FAD mouse brain. (FIG. 16B) High mag images showing that some the NeuroD1 converted neurons have the Tbr1 signal (which, when viewed in color, stained red) in frontal cortex (FCX) and parietal cortex (PCX).

FIGS. 17A-17B. Retro-orbital injection of AAV.PHP.eB-pGFAP::GFP. (FIG. 17A) Sagittal image at day 17 after injection showing the efficient infection throughout the brain. (FIG. 17B) High magnification image of different regions of brains showing specific expression of GFP in the astrocytes. Scale bar: 100 μm.

FIGS. 18A-18B. Retro-orbital injection of AAV.PHP.eB-pGFAP::Cre+FLEX-GFP. (FIG. 18A) Sagittal image at day 14 after injection showing the efficient infection throughout the brain. (FIG. 18B) High magnification image of different regions of brains showing expression of GFP in both astrocytes and neurons. Scale bar: 100 μm.

FIGS. 19A-19B. Retro-orbital injection of AAV.PHP.eB-pGFAP::Cre+FLEX-NeuroD1-GFP. (FIG. 19A) Sagittal image at day 14 after injection showing the efficient infection throughout the brain. (FIG. 19B) High magnification image of GFP and NeuroD1 signal at different regions of brains showing colocalization with neuronal marker NeuN. Scale bar: 100 μm.

FIG. 20. Diagram of a retro-orbital (r.o.) injection of a virus (e.g., AAV.PHP.eb-CAG::Flex-GFP) for global targeting of astrocytes and a cross between a Cre 77.6 mouse and a 5×FAD mouse to create a bigenic mouse (5×FAD^{+/-}/GFAP::Cre77.6^{+/-} mouse).

FIG. 21A-21B. Demonstration of global targeting of astrocytes in the biogenic (5×FAD^{+/-}/GFAP::Cre77.6^{+/-}) mouse. (FIG. 21A) Diagram of method used to targeting of astrocytes. (FIG. 21B) Image of mice brains following an retro-orbital (r.o.) injection of 2.0×10¹⁰ to 3.0×10¹⁰ genome copies/mouse of AAV.PHP.eb-CAG::Flex-GFP 11 DPI.

FIG. 22. Microscopy image demonstrates viral infection of the spinal cord cells by AAV.PHP.eb-CAG::Flex-GFP 11 DPI following an retro-orbital (r.o.) injection of 2.0×10¹⁰ to 3.0×10¹⁰ genome copies/mouse.

FIG. 23. Microscopy image demonstrates the detection of no obvious GFP signals in other organs 11 DPI following an retro-orbital injection of AAV.PHP.eb-CAG::Flex-GFP (2.0×10¹⁰ to 3.0×10¹⁰ genome copies/mouse).

FIG. 24. Microscopy image demonstrates global targeting of astrocytes in different regions of the brain by AAV.PHP.eb-CAG::Flex-GFP 11 DPI following an retro-orbital (r.o.) injection of AAV.PHP.eb-CAG::Flex-GFP (2.0×10¹⁰ to 3.0×10¹⁰ genome copies/mouse). OB=olfactory bulb; PiF=piriform cortex; MO=motor cortex; Str=striatum; SS=somatosensory cortex; VIS=visual cortex; Sub=subiculum; Hip=hippocampus; TH=thalamus; MB=midbrain; CB=cerebellum; and BS=brain stem.

FIG. 25A. Microscopy image demonstrates the conversion of astrocytes into neurons within the cerebrum 30 DPI of AAV.PHP.eb-CAG::Flex-ND1-P2A-GFP via retro-orbital injection (about 2.0×10¹⁰ genome copies/mouse). FIG. 25B. Microscopy image demonstrates no converted neurons within the spinal cord 30 DPI of AAV.PHP.eb-CAG::Flex-ND1-P2A-GFP via retro-orbital injection (about 2.0×10¹⁰ genome copies/mouse).

FIG. 26. Microscopy image demonstrates global conversion of astrocytes into neurons in different regions of the brain by AAV.PHP.eb-CAG::Flex-ND1-P2A-GFP 30 DPI following an retro-orbital (r.o.) injection of AAV.PHP.eb-CAG::Flex-ND1-P2A-GFP (about 2.0×10¹⁰ genome copies/mouse). OB=olfactory bulb; PiF=piriform cortex; MO=motor cortex; Str=striatum; SS=somatosensory cortex; VIS=visual cortex; Sub=subiculum; Hip=hippocampus; TH=thalamus; MB=midbrain; CB=cerebellum; and BS=brain stem.

FIG. 27. Microscopy image shows a direct comparison of different brain regions for mice (5×FAD^{+/-}/GFAP::Cre77.6^{+/-} mice) receiving control virus (AAV.PHP.eb-CAG::Flex-GFP; about 2.0×10¹⁰ genome copies/mouse) designated GFP Ctrl (top) or receiving NeuroD1 expressing virus (AAV.PHP.eb-CAG::Flex-ND1-P2A-GFP; about 2.0×10¹⁰ genome copies/mouse) designated NeuroD1 (bottom) 30 DPI following an retro-orbital (r.o.) injection. The control virus clearly infects astrocytes, while the virus driving NeuroD1 expression infects astrocytes and converts them into distinctive neurons. OB=olfactory bulb; PiF=piriform cortex; MO=motor cortex; Str=striatum; SS=somatosensory cortex; VIS=visual cortex; Sub=subiculum; Hip=hippocampus; TH=thalamus; MB=midbrain; CB=cerebellum; and BS=brain stem.

FIG. 28. A schematic illustration of the experimental design for confirming the ability of viruses designed to express NeuroD1 to improve memory in AD.

FIG. 29A-29B. Provides photographs of a Y maze for assessing memory in a mouse model for AD. FIG. 29A is the Y maze for the control group. FIG. 29B is the Y maze for the NeuroD1 group.

FIG. 30. Bar graphs of mouse arm entry (top) and alteration % (bottom) for male and female 5×FAD^{+/-}/GFAP::Cre77.6^{+/-} mice receiving control virus (AAV.PHP.eb-CAG::Flex-GFP virus designated GFP) or NeuroD1-expressing virus (AAV.PHP.eb-CAG::Flex-ND1-P2A-GFP designated NeuroD1) via retro-orbital injection of about 2.0×10¹⁰ genome copies/mouse.

FIG. 31. Line graph provides a normalized odor investigation time for odors 1, 2, 3, and 4. Results generated from an odor habituation assay performed as described elsewhere (Wesson et al., J. Neurosci., 30(2):505-514 (2010)) using 5×FAD^{+/-}/GFAP::Cre77.6^{+/-} mice receiving control virus (AAV.PHP.eb-CAG::Flex-GFP virus designated GFP) or NeuroD1-expressing virus (AAV.PHP.eb-CAG::Flex-ND1-P2A-GFP designated NeuroD1) via retro-orbital injection of about 2.0×10¹⁰ genome copies/mouse.

FIG. 32. Bar graphs demonstrate freezing percentages from a fear conditional memory test performed as described elsewhere (Choi et al., Mol. Brain, 9:72 (2016)) using 5×FAD^{+/-}/GFAP::Cre77.6^{+/-} mice receiving control virus (AAV.PHP.eb-CAG::Flex-GFP virus designated GFP) or NeuroD1-expressing virus (AAV.PHP.eb-CAG::Flex-ND1-P2A-GFP designated NeuroD1) via retro-orbital injection of about 2.0×10¹⁰ genome copies/mouse.

FIG. 33. Diagram of a Morris Water Maze for assessing spatial learning and memory.

FIG. 34A-34D. Results of the for the Morris Water Maze assessment performed using 5×FAD^{+/-}/GFAP::Cre77.6^{+/-} mice receiving control virus (AAV.PHP.eb-CAG::Flex-GFP virus designated GFP) or NeuroD1-expressing virus (AAV.PHP.eb-CAG::Flex-ND1-P2A-GFP designated NeuroD1) via retro-orbital injection of about 2.0×10¹⁰ genome copies/mouse. Diagram of the movement path (FIG. 34A). Bar graph of time in goal quadrant for control mice (GFP) compared with NeuroD1 mice (FIG. 34B). Line graph of Latency over 1, 2, 3, 4, and 5, days, for control mice (GFP) compared with NeuroD1 mice (FIG. 34C). Bar graph of the number of performed crossing between control mice (GFP) and NeuroD1 mice (FIG. 34D).

FIGS. 35A-35D. NeuroD1-converted neurons contribute to the memory improvement in AD mice. (FIG. 35A) Schematic illustration of the chemogenetic strategy to test whether converted neurons are directly responsible for fear memory recovery. (FIG. 35B) Immunostaining results showing that the DREADD receptors (hM4Di) are specifically expressed in the converted neurons (arrows in NeuroD1 row). Scale bar: 20 μm (FIG. 35C) The paradigm of fear conditioning memory test. Memory tests are performed at 2 months post AAV injection. The bottom diagram illustrates that the converted neurons are rapidly inhibited by the injection of clozapine-N-oxide (CNO). (FIG. 35D) Summary graph showing that fear conditioning memory enhancement in the NeuroD1-treated 5×FAD mice (NeuroD1, saline group) is abolished by the CNO administration (NeuroD1, CNO group). Data are shown as mean±SEM. ***p<0.001, one-way ANOVA with Turkey post-hoc tests.

### DETAILED DESCRIPTION

This document provides methods and materials involved in treating mammals having a neurological disorder in the brain (e.g., Alzheimer's disease). For example, this document provides methods and materials for administering a composition containing exogenous nucleic acid encoding a NeuroD1 polypeptide (or a biologically active fragment thereof) to a mammal having a neurological disorder in the brain.

Any appropriate mammal can be identified as having a neurological disorder (e.g., Alzheimer's disease) in the brain. For example, humans and other primates such as monkeys can be identified as having Alzheimer's disease.

In some cases, administration of a therapeutically effective amount of exogenous nucleic acid encoding a NeuroD1 polypeptide to a subject affected by a neurological disorder (e.g., Alzheimer's disease) in the brain mediates: the generation of new glutamatergic neurons by conversion of reactive astrocytes to glutamatergic neurons; reduction of the number of reactive astrocytes; survival of injured neurons including GABAergic and glutamatergic neurons; the generation of new non-reactive astrocytes; the reduction of reactivity of non-converted reactive astrocytes; and reintegration of blood vessels into the injured region. In some embodiments, administration of a therapeutically effective amount of exogenous nucleic acid encoding a NeuroD1 polypeptide to a subject affected by a neurological disorder (e.g., Alzheimer's disease) in the brain mediates: (1) the reduction in neurofibrillary tangles of hyperphosphorylated tau protein, (2) the reduction in aggregation of extracellular amyloid plaques, (3) the reduction of neuroinflammation, (4) the reduction of interleukin 1β (IL-1β) levels, (5) generating new glutamatergic neurons, (6) increasing the survival of GABAergic neurons, (7) generating new non-reactive astrocytes, (8) reducing the number of reactive astrocytes, and (9) improving memory.

In some cases, a method or composition provided herein reduces neurofibrillary tangles of hyperphosphorylated tau protein by between about 1% and 100% after administration of a composition provided herein. In some cases, a method or composition provided herein reduces neurofibrillary tangles of hyperphosphorylated tau protein by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after a composition provided herein.

In some cases, a method or composition provided herein reduces the aggregation of extracellular amyloid plaques by between about 1% and 100% after administration of a composition provided herein. In some cases, a method or composition provided herein reduces the aggregation of extracellular amyloid plaques by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein.

In some cases, a method or composition provided herein reduces neuroinflammation by between about 1% and 100% after administration of a composition provided herein. In some cases, a method or composition provided here in reduces neuroinflammation by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein.

In some cases, a method or composition provided herein reduces interleukin 1β (IL-1β) in the brain by between about 1% and 100% after administration of a composition provided herein. In some cases, a method or composition provided herein reduces interleukin 1β (IL-1β) in the brain by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein.

In some cases, a method or composition provided herein generates new glutamatergic neurons, increasing the number of glutamatergic neurons from a baseline level by between about 1% and 500% after administration of a composition provided herein. In some cases, a method or composition provided herein generates new glutamatergic neurons, increasing the number of glutamatergic neurons from a baseline level by between about 1% and 50%, between about 1% and 100%, between about 1% and 150%, between about 50% and 100%, between about 50% and 150%, between about 50% and 200%, between about 100% and 150%, between about 100% and 200%, between 100% and 250%, between about 150% and 200%, between about 150% and 250%, between about 150% and 300%, between 200% and 250%, between 200% and 300%, between 200% and 350%, between 250% and 300%, between 250% and 350%, between about 250% and 400%, between about 300% and 350%, between about 300% and 400%, between about 300% and 450%, between about 350% and 400%, between about 350% and 450%, between about 350% and 500%, between about 400% and 450%, between about 400% and 500%, or between about 450% and 500% after administration of a composition provided herein.

In some cases, a method or composition provided herein increases survival of GABAergic neurons by between about 1% and 100% after administration of a composition provided herein compared with no administration. In some cases, a method or composition provided herein increases survival of GABAergic neurons by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein compared with no administration. Any appropriate method can be used to assess increases in survival of GABAergic neurons. For example, immunostaining for γ-aminobutyric acid (GABA), GABA synthesizing enzyme glutamate decarboxylase 67 (GAD67), and/or parvalbumin (PV) can be performed to measure the number of GABAergic neurons. A decrease in the number of GABAergic neurons can indicate GABAergic neuronal loss. When the number remains unchanged, it can indicate that GABAergic neurons survive. An increase in the number of GABAergic neurons can indicate the occurrence of GABAergic regeneration.

In some cases, a method or composition provided herein generates new non-reactive astrocytes, increasing the number of new non-reactive astrocytes from a baseline level by between about 1% and 100% after administration of a composition provided herein. In some cases, a method or composition provided herein generates new non-reactive astrocytes, increasing the number of new non-reactive astrocytes from a baseline level by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In some cases, a method or composition provided herein reduces the number of reactive astrocytes by between about 1% and 100% after administration of a composition provided herein. In some cases, a method or composition provided herein reduces the number of reactive astrocytes by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein.

In some cases, a method or composition provided herein improves the memory evaluation characteristics of a mammal by between about 1% and 100% after administration of a composition provided herein. In some cases, a method or composition provided herein improves the memory evaluation characteristics of a mammal by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% administration of a composition provided herein.

In some cases, administration of a therapeutically effective amount of exogenous nucleic acid encoding a NeuroD1 polypeptide to a subject affected by a neurological disorder (e.g., Alzheimer's disease) in the brain can re-introduce homeostasis, contribute to the clearing of the plaques, and/or improve brain vascularization and blood flow.

In some cases, administration of a therapeutically effective amount of exogenous nucleic acid encoding a NeuroD1 polypeptide to a subject affected by a neurological disorder (e.g., Alzheimer's disease) in the brain mediates: reduced inflammation at the injury site; reduced neuroinhibition at the injury site; re-establishment of normal microglial morphology at the injury site; re-establishment of neural circuits at the injury site; increased blood vessels at the injury site; re-establishment of blood-brain-barrier at the injury site; re-establishment of normal tissue structure at the injury site; and improvement of motor deficits due to the disruption of normal blood flow.

In some cases, administration of a therapeutically effective amount of exogenous nucleic acid encoding a NeuroD1 polypeptide to ameliorate the effects of a neurological disorder (e.g., Alzheimer's disease) in the brain in an individual subject in need thereof has greater beneficial effects when administered to reactive astrocytes than to quiescent astrocytes.

NeuroD1 treatment can be administered to the region of injury as diagnosed by MRI. Electrophysiology can assess functional changes in neural firing as caused by neural cell death or injury. Non-invasive methods to assay neural damage include electroencephalogram (EEG). Disruption of blood flow to a point of injury may be non-invasively assayed via Near Infrared Spectroscopy and functional magnetic resonance imaging (fMRI). Blood flow within the region may either be increased, as seen in aneurysms, or decreased, as seen in ischemia. Injury to the CNS caused by disruption of blood flow additionally causes short-term and long-term changes to tissue structure that can be used to diagnose a point of injury. In the short term, injury will cause localized swelling. In the long term, cell death will cause points of tissue loss. Non-invasive methods to assay structural changes caused by tissue death include magnetic resonance imaging (MRI), positron emission tomography (PET) scan, computerized axial tomography (CAT) scan, or ultrasound. These methods may be used singularly or in any combination to pinpoint the focus of injury.

As described above, non-invasive methods to assay structural changes caused by tissue death include MRI, CAT scan, or ultrasound. Functional assays may include EEG recordings. In some embodiments of the methods for treating a neurological disorder as described herein, NeuroD1 is administered as an expression vector containing a DNA sequence encoding NeuroD1.

In some embodiments of the methods for treating a neurological disorder as described herein, a viral vector (e.g., an AAV) including a nucleic acid encoding NeuroD1 is delivered by injection into the brain of a subject, such as stereotaxic intracranial injection or retro-orbital injection. In some cases, the composition containing the adeno-associated virus encoding NeuroD1 is administered to the brain using two more intracranial injections at the same location in the brain. In some cases, the composition containing the adeno-associated virus encoding NeuroD1 is administered to the brain using two more intracranial injections at two or more different locations in the brain.

The term “expression vector” refers to a recombinant vehicle for introducing a nucleic acid encoding NeuroD1 into a host cell in vitro or in vivo where the nucleic acid is expressed to produce NeuroD1. In particular embodiments, an expression vector including SEQ ID NO:1 or 3 or a substantially identical nucleic acid sequence is expressed to produce NeuroD1 in cells containing the expression vector. The term “recombinant” is used to indicate a nucleic acid construct in which two or more nucleic acids are linked and which are not found linked in nature. Expression vectors include, but are not limited to, plasmids, viruses, BACs, and YACs. Particular viral expression vectors illustratively include those derived from an adenovirus, an adeno-associated virus, a retrovirus, and a lentivirus.

This document describes material and methods for treating a neurological disorder (e.g., Alzheimer's disease) in a subject in need thereof according to the methods described which include providing a viral vector comprising a nucleic acid encoding NeuroD1; and delivering the viral vector to the brain of the subject, whereby the viral vector infects glial cells of the central nervous system, respectively, producing infected glial cells and whereby exogenous NeuroD1 polypeptide (e.g., exogenous nucleic acid encoding a NeuroD1 polypeptide) is expressed in the infected glial cells at a therapeutically effective level, wherein the expression of NeuroD1 in the infected cells results in a greater number of neurons in the subject compared to an untreated subject having the same neurological condition, whereby the neurological disorder is treated. In addition to the generation of new neurons, the number of reactive glial cells will also be reduced, resulting in less neuroinhibitory factors released, less neuroinflammation, and more blood vessels that are also evenly distributed, thereby making a local environment more permissive to neuronal growth or axon penetration, hence alleviating neurological conditions.

In some cases, adeno-associated vectors are particularly useful in methods described herein and will infect both dividing and non-dividing cells, at an injection site. Adeno-associated viruses (AAV) are ubiquitous, noncytopathic, replication-incompetent members of ssDNA animal virus of parvoviridae family. According to some aspects, any of various recombinant adeno-associated viruses, such as serotypes 1-9, can be used as described herein. In some cases, an AAV-PHP.eb is used to administer the exogenous nucleic acid encoding a NeuroD1 polypeptide (or a biologically active fragment thereof).

A “FLEX” switch approach is used to express NeuroD1 in infected cells according to some aspects described herein. The terms “FLEX” and “flip-excision” are used interchangeably to indicate a method in which two pairs of heterotypic, antiparallel loxP-type recombination sites are disposed on either side of an inverted NeuroD1 coding sequence which first undergo an inversion of the coding sequence followed by excision of two sites, leading to one of each orthogonal recombination site oppositely oriented and incapable of further recombination, achieving stable inversion, see for example Schnutgen et al., Nature Biotechnology, 21:562-565 (2003); and Atasoy et al., J. Neurosci., 28:7025-7030 (2008). Since the site-specific recombinase under control of a glial cell-specific promoter will be strongly expressed in glial cells, including reactive astrocytes, NeuroD1 will also be expressed in glial cells, including reactive astrocytes. Then, when the stop codon in front of NeuroD1 is removed from recombination, the constitutive or neuron-specific promoter will drive high expression of NeuroD1, allowing reactive astrocytes to be converted into functional neurons.

According to particular aspects, NeuroD1 is administered to a subject in need thereof by administration of (1) an adeno-associated virus expression vector including a DNA sequence encoding a site-specific recombinase under transcriptional control of an astrocyte-specific promoter such as GFAP or S100b or Aldh1L1; and (2) an adeno-associated virus expression vector including a DNA sequence encoding NeuroD1 under transcriptional control of a ubiquitous (constitutive) promoter or a neuron-specific promoter wherein the DNA sequence encoding NeuroD1 is inverted and in the wrong orientation for expression of NeuroD1 until the site-specific recombinase inverts the inverted DNA sequence encoding NeuroD1, thereby allowing expression of NeuroD1.

Site-specific recombinases and their recognition sites include, for example, Cre recombinase along with recognition sites loxP and lox2272 sites, or FLP-FRT recombination, or their combinations.

A composition including an exogenous nucleic acid sequence encoding a NeuroD1 polypeptide (e.g., an AAV encoding a NeuroD1 polypeptide) can be administered to a mammal once or multiple times (e.g., two, three, four, five, or more times).

A composition including an exogenous nucleic acid sequence encoding a NeuroD1 polypeptide (e.g., an AAV encoding a NeuroD1 polypeptide) can be formulated into a pharmaceutical composition for administration into a mammal. For example, a therapeutically effective amount of the composition including an exogenous nucleic acid encoding a NeuroD1 polypeptide (or a biologically active fragment thereof) can be formulated with one or more pharmaceutically acceptable carriers (additives) and/or diluents. A pharmaceutical composition including an exogenous NeuroD1 (e.g., an AAV encoding NeuroD1) can be formulated for various routes of administration—for example, for oral administration as a capsule, a liquid or the like. In some cases, a viral vector (e.g., AAV) having an exogenous nucleic acid encoding a NeuroD1 polypeptide (or a biologically active fragment thereof) is administered parenterally, preferably by intravenous injection or intravenous infusion. The administration can be, for example, by intravenous infusion, for example for 60 minutes, for 30 minutes or for 15 minutes. In some cases, the administration can be between 1 minute and 60 minutes. In some cases, the administration can be between 1 minute and 5 minutes, between 1 minute and 10 minutes, between 1 minute and 15 minutes, between 5 minutes and 10 minutes, between 5 minutes and 15 minutes, between 5 minutes and 20 minutes, between 10 minutes and 15 minutes, between 10 minutes and 20 minutes, between 10 minutes and 25 minutes, between 15 minutes and 20 minutes, between 15 minutes and 25 minutes, between 15 minutes and 30 minutes, between 20 minutes and 25 minutes, between 20 minutes and 30 minutes, between 20 minutes and 35 minutes, between 25 minutes and 30 minutes, between 25 minutes and 35 minutes, between 25 minutes and 40 minutes, between 30 minutes and 35 minutes, between 30 minutes and 40 minutes, between 30 minutes and 45 minutes, between 35 minutes and 40 minutes, between 35 minutes and 45 minutes, between 35 minutes and 50 minutes, between 40 minutes and 45 minutes, between 40 minutes and 50 minutes, between 40 minutes and 55 minutes, between 45 minutes and 50 minutes, between 45 minutes and 55 minutes, between 45 minutes and 60 minutes, between 50 minutes and 55 minutes, between 50 minutes and 60 minutes, or between 55 minutes and 60 minutes. In some cases, the viral vector (e.g., AAV encoding NeuroD1) is administered locally by injection to the brain during a surgery. Compositions which are suitable for administration by injection and/or infusion include solutions and dispersions, and powders from which corresponding solutions and dispersions can be prepared. Such compositions will comprise the viral vector and at least one suitable pharmaceutically acceptable carrier. Suitable pharmaceutically acceptable carriers for intravenous administration include, but not limited to, bacterostatic water, Ringer's solution, physiological saline, phosphate buffered saline (PBS), and Cremophor EL™. Sterile compositions for the injection and/or infusion can be prepared by introducing the viral vector (e.g., AAV encoding NeuroD1) in the required amount into an appropriate carrier, and then sterilizing by filtration. Compositions for administration by injection or infusion should remain stable under storage conditions after their preparation over an extended period of time. The compositions can contain a preservative for this purpose. Suitable preservatives include, but not limited to, chlorobutanol, phenol, ascorbic acid, and thimerosal.

A pharmaceutical composition can be formulated for administration in solid or liquid form including, without limitation, sterile solutions, suspensions, sustained-release formulations, tablets, capsules, pills, powders, and granules. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

Additional pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

As used herein, the term “adeno-associated virus particle” refers to packaged capsid forms of the AAV virus that transmits its nucleic acid genome to cells.

An effective amount of composition containing an exogenous nucleic acid encoding a NeuroD1 polypeptide (or a biologically active fragment thereof) can be any amount that ameliorates the symptoms of the neurological disorder within a mammal (e.g., a human) without producing severe toxicity to the mammal. For example, an effective amount of adeno-associated virus encoding a NeuroD1 polypeptide can be a concentration from about 10¹⁰ to 10¹⁴ adeno-associated virus particles/mL. In some cases, an effective amount of adeno-associated virus encoding a NeuroD1 polypeptide can be between 10¹⁰ adeno-associated virus particles/mL and 10¹¹ adeno-associated virus particles/mL, between 10¹⁰ adeno-associated virus particles/mL and 10¹² adeno-associated virus particles/mL, between 10¹⁰ adeno-associated virus particles/mL and 10¹³ adeno-associated virus particles/mL, between 10¹¹ adeno-associated virus particles/mL and 10¹² adeno-associated virus particles/mL, between 10¹¹ adeno-associated virus particles/mL and 10¹³ adeno-associated virus particles/mL, between 10¹¹ adeno-associated virus particles/mL and 10¹⁴ adeno-associated virus particles/mL, between 10¹² adeno-associated virus particles/mL and 10¹³ adeno-associated virus particles/mL, between 10¹² adeno-associated virus particles/mL and 10¹⁴ adeno-associated virus particles/mL, or between 10¹³ adeno-associated virus particles/mL and 10¹⁴ adeno-associated virus particles/mL. If a particular mammal fails to respond to a particular amount, then the amount of the AAV encoding a NeuroD1polypeptide can be increased. Factors that are relevant to the amount of viral vector (e.g., an AAV having an exogenous nucleic acid encoding a NeuroD1 polypeptide (or a biologically active fragment thereof)) to be administered are, for example, the route of administration of the viral vector, the nature and severity of the disease, the disease history of the patient being treated, and the age, weight, height, and health of the patient to be treated. In some cases, the expression level of the transgene, which is required to achieve a therapeutic effect, the immune response of the patient, as well as the stability of the gene product are relevant for the amount to be administered. In some cases, the administration of the viral vector (e.g., an AAV having an exogenous nucleic acid encoding a NeuroD1 polypeptide (or a biologically active fragment thereof)) occurs in an amount which leads to a complete or substantially complete healing of the dysfunction or disease of the brain.

In some cases, an effective amount of composition containing an exogenous nucleic acid encoding a NeuroD1 polypeptide (or a biologically active fragment thereof) can be any amount administered at a controlled flow rate of about 0.1 μL/minute to about 5 μL/minute.

In some cases, the controlled flow rate is between 0.1 μL/minute and 0.2 μL/minute, between 0.1 μL/minute and 0.3 μL/minute, between 0.1 μL/minute and 0.4 μL/minute, between 0.2 μL/minute and 0.3 μL/minute, between 0.2 μL/minute and 0.4 μL/minute, between 0.2 μL/minute and 0.5 μL/minute, between 0.3 μL/minute and 0.4 μL/minute, between 0.3 μL/minute and 0.5 μL/minute, between 0.3 μL/minute and 0.6 μL/minute, between 0.4 μL/minute and 0.5 μL/minute, between 0.4 μL/minute and 0.6 μL/minute, between 0.4 μL/minute and 0.7 μL/minute, between 0.5 μL/minute and 0.6 μL/minute, between 0.5 μL/minute and 0.7 μL/minute, between 0.5 μL/minute and 0.8 μL/minute, between 0.6 μL/minute and 0.7 μL/minute, between 0.6 μL/minute and 0.8 μL/minute, between 0.6 μL/minute and 0.9 μL/minute, between 0.7 μL/minute and 0.8 μL/minute, between 0.7 μL/minute and 0.9 μL/minute, between 0.7 μL/minute and 1.0 μL/minute, between 0.8 μL/minute and 0.9 μL/minute, between 0.8 μL/minute and 1.0 μL/minute, between 0.8 μL/minute and 1.1 μL/minute, between 0.9 μL/minute and 1.0 μL/minute, between 0.9 μL/minute and 1.1 μL/minute, between 0.9 μL/minute and 1.2 μL/minute, between 1.0 μL/minute and 1.1 μL/minute, between 1.0 μL/minute and 1.2 μL/minute, between 1.0 μL/minute and 1.3 μL/minute, between 1.1 μL/minute and 1.2 μL/minute, between 1.1 μL/minute and 1.3 μL/minute, between 1.1 μL/minute and 1.4 μL/minute, between 1.2 μL/minute and 1.3 μL/minute, between 1.2 μL/minute and 1.4 μL/minute, between 1.2 μL/minute and 1.5 μL/minute, between 1.3 μL/minute and 1.4 μL/minute, between 1.3 μL/minute and 1.5 μL/minute, between 1.3 μL/minute and 1.6 μL/minute, between 1.4 μL/minute and 1.5 μL/minute, between 1.4 μL/minute and 1.6 μL/minute, between 1.4 μL/minute and 1.7 μL/minute, between 1.5 μL/minute and 1.6 μL/minute, between 1.5 μL/minute and 1.7 μL/minute, between 1.5 μL/minute and 1.8 μL/minute, between 1.6 μL/minute and 1.7 μL/minute, between 1.6 μL/minute and 1.8 μL/minute, between 1.6 μL/minute and 1.9 μL/minute, between 1.7 μL/minute and 1.8 μL/minute, between 1.7 μL/minute and 1.9 μL/minute, between 1.7 μL/minute and 2.0 μL/minute, between 1.8 μL/minute and 1.9 μL/minute, between 1.8 μL/minute and 2.0 μL/minute, between 1.8 μL/minute and 2.1 μL/minute, between 1.9 μL/minute and 2.0 μL/minute, between 1.9 μL/minute and 2.1 μL/minute, between 1.9 μL/minute and 2.2 μL/minute, between 2.0 μL/minute and 2.1 μL/minute, between 2.0 μL/minute and 2.2 μL/minute, between 2.0 μL/minute and 2.3 μL/minute, between 2.1 μL/minute and 2.2 μL/minute, between 2.1 μL/minute and 2.3 μL/minute, between 2.1 μL/minute and 2.4 μL/minute, between 2.2 μL/minute and 2.3 μL/minute, between 2.2 μL/minute and 2.4 μL/minute, between 2.2 μL/minute and 2.5 μL/minute, between 2.3 μL/minute and 2.4 μL/minute, between 2.3 μL/minute and 2.5 μL/minute, between 2.3 μL/minute and 2.6 μL/minute, between 2.4 μL/minute and 2.5 μL/minute, between 2.4 μL/minute and 2.6 μL/minute, between 2.4 μL/minute and 2.7 μL/minute, between 2.5 μL/minute and 2.6 μL/minute, between 2.5 μL/minute and 2.7 μL/minute, between 2.5 μL/minute and 2.8 μL/minute, between 2.6 μL/minute and 2.7 μL/minute, between 2.6 μL/minute and 2.8 μL/minute, between 2.6 μL/minute and 2.9 μL/minute, between 2.7 μL/minute and 2.8 μL/minute, between 2.7 μL/minute and 2.9 μL/minute, between 2.7 μL/minute and 3.0 μL/minute, between 2.8 μL/minute and 2.9 μL/minute, between 2.8 μL/minute and 3.0 μL/minute, between 2.8 μL/minute and 3.1 μL/minute, between 2.9 μL/minute and 3.0 μL/minute, between 2.9 μL/minute and 3.1 μL/minute, between 2.9 μL/minute and 3.2 μL/minute, between 3.0 μL/minute and 3.1 μL/minute, between 3.0 μL/minute and 3.2 μL/minute, between 3.0 μL/minute and 3.3 μL/minute, between 3.1 μL/minute and 3.2 μL/minute, between 3.1 μL/minute and 3.3 μL/minute, between 3.1 μL/minute and 3.4 μL/minute, between 3.2 μL/minute and 3.3 μL/minute, between 3.2 μL/minute and 3.4 μL/minute, between 3.2 μL/minute and 3.5 μL/minute, between 3.3 μL/minute and 3.4 μL/minute, between 3.3 μL/minute and 3.5 μL/minute, between 3.3 μL/minute and 3.6 μL/minute, between 3.4 μL/minute and 3.5 μL/minute, between 3.4 μL/minute and 3.6 μL/minute, between 3.4 μL/minute and 3.7 μL/minute, between 3.5 μL/minute and 3.6 μL/minute, between 3.5 μL/minute and 3.7 μL/minute, between 3.5 μL/minute and 3.8 μL/minute, between 3.6 μL/minute and 3.7 μL/minute, between 3.6 μL/minute and 3.8 μL/minute, between 3.6 μL/minute and 3.9 μL/minute, between 3.7 μL/minute and 3.8 μL/minute, between 3.7 μL/minute and 3.9 μL/minute, between 3.7 μL/minute and 4.0 μL/minute, between 3.8 μL/minute and 3.9 μL/minute, between 3.8 μL/minute and 4.0 μL/minute, between 3.8 μL/minute and 4.1 μL/minute, between 3.9 μL/minute and 4.0 μL/minute, between 3.9 μL/minute and 4.1 μL/minute, between 3.9 μL/minute and 4.2 μL/minute, between 4.0 μL/minute and 4.1 μL/minute, between 4.0 μL/minute and 4.2 μL/minute, between 4.0 μL/minute and 4.3 μL/minute, between 4.1 μL/minute and 4.2 μL/minute, between 4.1 μL/minute and 4.3 μL/minute, between 4.1 μL/minute and 4.4 μL/minute, between 4.2 μL/minute and 4.3 μL/minute, between 4.2 μL/minute and 4.4 μL/minute, between 4.2 μL/minute and 4.5 μL/minute, between 4.3 μL/minute and 4.4 μL/minute, between 4.3 μL/minute and 4.5 μL/minute, between 4.3 μL/minute and 4.6 μL/minute, between 4.4 μL/minute and 4.5 μL/minute, between 4.4 μL/minute and 4.6 μL/minute, between 4.4 μL/minute and 4.7 μL/minute, between 4.5 μL/minute and 4.6 μL/minute, between 4.5 μL/minute and 4.7 μL/minute, between 4.5 μL/minute and 4.8 μL/minute, between 4.6 μL/minute and 4.7 μL/minute, between 4.6 μL/minute and 4.8 μL/minute, between 4.6 μL/minute and 4.9 μL/minute, between 4.7 μL/minute and 4.8 μL/minute, between 4.7 μL/minute and 4.9 μL/minute, between 4.7 μL/minute and 5.0 μL/minute, 4.8 μL/minute and 4.9 μL/minute, between 4.8 μL/minute and 5.0 μL/minute, or between 4.9 μL/minute and 5.0 μL/minute.

The viral vector (e.g., an AAV having a nucleic acid encoding a NeuroD1 polypeptide (or a biologically active fragment thereof)) can be administered in an amount corresponding to a dose of virus in the range of about 1.0×10¹⁰ to about 1.0×10¹⁴ vg/kg (virus genomes per kg body weight). In some cases, the viral vector (e.g., an AAV having a nucleic acid encoding a NeuroD1 polypeptide (or a biologically active fragment thereof)) can be administered in amount corresponding to a dose of virus in the range of about 1.0×10¹¹ to about 1.0×10¹² vg/kg, a range of about 5.0×10¹¹ to about 5.0×10¹² vg/kg, or a range of about 1.0×10¹² to about 5.0×10¹¹. In some cases, the viral vector (e.g., an AAV having a nucleic acid encoding a NeuroD1 polypeptide (or a biologically active fragment thereof)) is administered in an amount corresponding to a dose of about 2.5×10¹² vg/kg. In some cases, the effective amount of the viral vector (e.g., an AAV having a nucleic acid encoding a NeuroD1 polypeptide (or a biologically active fragment thereof)) can be a volume of about 1 μL to about 500 μL, corresponding to the volume for the vg/kg (virus genomes per kg body weight) doses described herein.

In some cases, the effective volume administered of the viral vector is between 1 μL and 25 μL, between 1 μL and 50 μL, between 1 μL and 75 μL, between 25 μL and 50 μL, between 25 μL and 75 μL, between 25 μL and 100 μL, between 50 μL and 75 μL, between 50 μL and 100 μL, between 50 μL and 125 μL, between 75 μL and 100 μL, between 75 μL and 125 μL, between 75 μL and 150 μL, between 100 μL and 125 μL, between 100 μL and 150 μL, between 100 μL and 175 μL, between 125 μL and 150 μL, between 125 μL and 175 μL, between 125 μL and 200 μL, between 150 μL and 175 μL, between 150 μL and 200 μL, between 150 μL and 225 μL, between 175 μL and 200 μL, between 175 μL and 225 μL, between 175 μL and 250 μL, between 200 μL and 225 μL, between 200 μL and 250 μL, between 200 μL and 275 μL, between 225 μL and 250 μL, between 225 μL and 275 μL, between 225 μL and 300 μL, between 250 μL and 275 μL, between 250 μL and 300 μL, between 250 μL and 325 μL, between 275 μL and 300 μL, between 275 μL and 325 μL, between 275 μL and 350 μL, between 300 μL and 325 μL, between 300 μL and 350 μL, between 300 μL and 375 μL, between 325 μL and 350 μL, between 325 μL and 375 μL, between 325 μL and 400 μL, between 350 μL and 375 μL, between 350 μL and 400 μL, between 350 μL and 425 μL, between 375 μL and 400 μL, between 375 μL and 425 μL, between 375 μL and 450 μL, between 400 μL and 425 μL, between 400 μL and 450 μL, between 400 μL and 475 μL, between 425 μL and 450 μL, between 425 μL and 475 μL, between 425 μL and 500 μL, between 450 μL and 475 μL, between 450 μL and 500 μL, or between 475 μL and 500 μL.

In some cases, the amount of the viral vector to be administered (e.g., an AAV having a exogenous nucleic acid encoding a NeuroD1 polypeptide (or a biologically active fragment thereof)) is adjusted according to the strength of the expression of one or more transgenes (e.g., NeuroD1).

In some cases, an adeno-associated virus vector including a nucleic acid encoding NeuroD1 under transcriptional control of a ubiquitous (constitutive) promoter or a neuron-specific promoter wherein the DNA sequence encoding NeuroD1 is inverted and in the wrong orientation for expression of NeuroD1 and further includes sites for recombinase activity by a site specific recombinase, until the site-specific recombinase inverts the inverted DNA sequence encoding NeuroD1, thereby allowing expression of NeuroD1, is delivered by stereotactic injection into the brain of a subject along with an adeno-associated virus encoding a site specific recombinase.

In some cases, an adeno-associated virus vector including a nucleic acid encoding NeuroD1 under transcriptional control of a ubiquitous (constitutive) promoter or a neuron-specific promoter wherein the DNA sequence encoding NeuroD1 is inverted and in the wrong orientation for expression of NeuroD1 and further includes sites for recombinase activity by a site specific recombinase, until the site-specific recombinase inverts the inverted DNA sequence encoding NeuroD1, thereby allowing expression of NeuroD1, is delivered by stereotactic injection into the brain of a subject along with an adeno-associated virus encoding a site specific recombinase in the region of or at the site of disruption of normal blood flow in the CNS according to some aspects. Optionally, the site of stereotactic injection is in or near a glial scar caused by disruption of normal blood flow in the CNS.

In some cases, the site-specific recombinase is Cre recombinase, and the sites for recombinase activity are recognition sites loxP and lox2272 sites.

In some cases, NeuroD1 treatment of a subject is monitored during or after treatment to monitor progress and/or final outcome of the treatment. Post-treatment assays for successful neuronal cell integration and restoration of tissue microenvironment is diagnosed by restoration or near-restoration of normal electrophysiology, blood flow, tissue structure, and function. Non-invasive methods to assay neural function include EEG. Blood flow may be non-invasively assayed via Near Infrared Spectroscopy and fMRI. Non-invasive methods to assay tissue structure include MRI, CAT scan, PET scan, or ultrasound. Behavioral assays may be used to non-invasively assay for restoration of brain function. The behavioral assay should be matched to the loss of function caused by original brain injury. For example, if injury caused paralysis, the patient's mobility and limb dexterity should be tested. If injury caused loss or slowing of speech, patient's ability to communicate via spoken word should be assayed. Restoration of normal behavior post NeuroD1 treatment indicates successful creation and integration of effective neuronal circuits. These methods may be used singularly or in any combination to assay for neural function and tissue health. Assays to evaluate treatment may be performed at any point, such as 1 day, 2 days, 3 days, one week, 2 weeks, 3 weeks, one month, two months, three months, six months, one year, or later, after NeuroD1 treatment. Such assays may be performed prior to NeuroD1 treatment in order to establish a baseline comparison if desired.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Asubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W. H. Freeman & Company, 2004; Engelke, D. R., RNA Interference (RNAi): Nuts and Bolts of RNAi Technology, DNA Press LLC, Eagleville, P A, 2003; Herdewijn, p. (Ed.), Oligonucleotide Synthesis: Methods and Applications, Methods in Molecular Biology, Humana Press, 2004; A. Nagy, M. Gertsenstein, K. Vintersten, R. Behringer, Manipulating the Mouse Embryo: A Laboratory Manual, Cold Spring Harbor Laboratory Press, 3rd Ed.; Dec. 15, 2002,ISBN-10:0879695919; Kursad Turksen (Ed.), Embryonic Stem Cells: Methods and Protocols in Methods in Molecular Biology, 2002; 185, Human Press: Current Protocols in Stem Cell Biology, ISBN:9780470151808.

As used herein, the singular terms “a,” “an,” and “the” are not intended to be limiting and include plural referents unless explicitly stated otherwise or the context clearly indicates otherwise.

As used herein, the term “NeuroD1 protein” refers to a bHLH proneural transcription factor involved in embryonic brain development and in adult neurogenesis, see Cho et al., Mol. Neurobiol., 30:35-47 (2004); Kuwabara et al., Nature Neurosci., 12:1097-1105 (2009); and Gao et al., Nature Neurosci., 12:1090-1092 (2009). NeuroD1 is expressed late in development, mainly in the nervous system and is involved in neuronal differentiation, maturation, and survival.

The terms “NeuroD1 nucleic acid” or “exogenous NeuroD1 nucleic acid” encompass a nucleic acid encoding a NeuroD1 polypeptide (or a biologically active fragment thereof), nucleic acid encoding a human NeuroD1 protein identified herein as SEQ ID NO:2, and nucleic acid encoding a mouse NeuroD1 protein identified herein as SEQ ID NO:4. In addition to the NeuroD1 protein of SEQ ID NO:2 and SEQ ID NO:4, the term “NeuroD1 protein” encompasses variants of a NeuroD1 protein, such as variants of SEQ ID NO:2 and SEQ ID NO:4, which may be included in the methods described herein. As used herein, the term “variant” refers to naturally occurring genetic variations and recombinantly prepared variations, each of which contain one or more changes in its amino acid sequence compared to a reference NeuroD1 protein, such as SEQ ID NO:2 or SEQ ID NO:4. Such changes include those in which one or more amino acid residues have been modified by amino acid substitution, addition, or deletion. The term “variant” encompasses orthologs of human NeuroD1, including for example mammalian and bird NeuroD1, such as, but not limited to NeuroD1 orthologs from a non-human primate, cat, dog, sheep, goat, horse, cow, pig, bird, poultry animal and rodent such as but not limited to mouse and rat. In a non-limiting example, mouse NeuroD1, exemplified herein as the amino acid sequence of SEQ ID NO:4, is an ortholog of human NeuroD1.

In some cases, preferred variants have at least 75%, 80%, 85%, 90%, 91%, 92%, 93%, 94%, 95%, 96%, 97%, 98%, or 99% identity to SEQ ID NO:2 or SEQ ID NO:4.

Mutations can be introduced using standard molecular biology techniques, such as site-directed mutagenesis and PCR-mediated mutagenesis. One of skill in the art will recognize that one or more amino acid mutations can be introduced without altering the functional properties of the NeuroD1 protein. For example, one or more amino acid substitutions, additions, or deletions can be made without altering the functional properties of the NeuroD1 protein of SEQ ID NO:2 or 4.

Conservative amino acid substitutions can be made in a NeuroD1 protein to produce a NeuroD1 protein variant. Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic, and hydrophilic. A conservative substitution is a substitution of one amino acid having a specified structural or functional characteristic for another amino acid having the same characteristic. Acidic amino acids include aspartate and glutamate; basic amino acids include histidine, lysine, and arginine; aliphatic amino acids include isoleucine, leucine, and valine; aromatic amino acids include phenylalanine, glycine, tyrosine, and tryptophan; polar amino acids include aspartate, glutamate, histidine, lysine, asparagine, glutamine, arginine, serine, threonine, and tyrosine; and hydrophobic amino acids include alanine, cysteine, phenylalanine, glycine, isoleucine, leucine, methionine, proline, valine, and tryptophan; and conservative substitutions include substitution among amino acids within each group. Amino acids may also be described in terms of relative size; alanine, cysteine, aspartate, glycine, asparagine, proline, threonine, serine, and valine are each typically considered to be small.

NeuroD1 variants can include synthetic amino acid analogs, amino acid derivatives, and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, PNAS, 87:2264-2268 (1990), modified as in Karlin and Altschul, PNAS, 90:5873-5877 (1993). Such an algorithm is incorporated into the NBLAST and XBLAST programs of Altschul et al., J. Mol. Biol., 215:403 (1990). BLAST nucleotide searches are performed with the NBLAST nucleotide program parameters set, e.g., for score=100, wordlength=12 to obtain nucleotide sequences homologous to a nucleic acid molecule described herein.

BLAST protein searches are performed with the)(BLAST program parameters set, e.g., to score 50, wordlength=3 to obtain amino acid sequences homologous to a protein molecule described herein. To obtain gapped alignments for comparison purposes, Gapped BLAST are utilized as described in Altschul et al., Nucleic Acids Res., 25:3389-3402 (1997). Alternatively, PSI BLAST is used to perform an iterated search which detects distant relationships between molecules. When utilizing BLAST, Gapped BLAST, and PSI Blast programs, the default parameters of the respective programs (e.g., of XBLAST and NBLAST) are used (see, e.g., the NCBI website).

Another preferred, non-limiting example of a mathematical algorithm utilized for the comparison of sequences is the algorithm of Myers and Miller, CABIOS, 4:11-17 (1988). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

The term “NeuroD1 protein” encompasses fragments of the NeuroD1 protein, such as fragments of SEQ ID NOs:2 and 4 and variants thereof, operable in methods and compositions described herein.

NeuroD1 proteins and nucleic acids may be isolated from natural sources, such as the brain of an organism or cells of a cell line which expresses NeuroD1. Alternatively, NeuroD1 protein or nucleic acid may be generated recombinantly, such as by expression using an expression construct, in vitro or in vivo. NeuroD1 proteins and nucleic acids may also be synthesized by well-known methods.

NeuroD1 included in methods and compositions can be produced using recombinant nucleic acid technology. Recombinant NeuroD1 production includes introducing a recombinant expression vector encompassing a DNA sequence encoding NeuroD1 into a host cell.

A nucleic acid sequence encoding NeuroD1 introduced into a host cell to produce NeuroD1 according to some embodiments encodes SEQ ID NO:2, SEQ ID NO:4, or a variant thereof.

In some cases, the nucleic acid sequence identified herein as SEQ ID NO:1 encodes SEQ ID NO:2 and is included in an expression vector and expressed to produce NeuroD1. According to some aspects, the nucleic acid sequence identified herein as SEQ ID NO:3 encodes SEQ ID NO:4 and is included in an expression vector and expressed to produce NeuroD1.

It is appreciated that due to the degenerate nature of the genetic code, nucleic acid sequences substantially identical to SEQ ID NOs:1 and 3 encode NeuroD1 and variants of NeuroD1, and that such alternate nucleic acids may be included in an expression vector and expressed to produce NeuroD1 and variants of NeuroD1. One of skill in the art will appreciate that a fragment of a nucleic acid encoding NeuroD1 protein can be used to produce a fragment of a NeuroD1 protein.

An expression vector can contain a nucleic acid that includes a segment encoding a polypeptide of interest operably linked to one or more regulatory elements that provide for transcription of the segment encoding the polypeptide of interest. The term “operably linked” as used herein refers to a nucleic acid in functional relationship with a second nucleic acid. The term “operably linked” encompasses functional connection of two or more nucleic acid molecules, such as a nucleic acid to be transcribed and a regulatory element. The term “regulatory element” as used herein refers to a nucleotide sequence which controls some aspect of the expression of an operably linked nucleic acid. Exemplary regulatory elements include an enhancer, such as, but not limited to: woodchuck hepatitis virus posttranscriptional regulatory element (WPRE); an internal ribosome entry site (IRES) or a 2A domain; an intron; an origin of replication; a polyadenylation signal (pA); a promoter; a transcription termination sequence; and an upstream regulatory domain, which contribute to the replication, transcription, post-transcriptional processing of an operably linked nucleic acid sequence. Those of ordinary skill in the art are capable of selecting and using these and other regulatory elements in an expression vector with no more than routine experimentation.

The term “promoter” as used herein refers to a DNA sequence operably linked to a nucleic acid sequence to be transcribed such as a nucleic acid sequence encoding NeuroD1. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors.

As will be recognized by the skilled artisan, the 5′ non-coding region of a gene can be isolated and used in its entirety as a promoter to drive expression of an operably linked nucleic acid. Alternatively, a portion of the 5′ non-coding region can be isolated and used to drive expression of an operably linked nucleic acid. In general, about 500-6000 bp of the 5′ non-coding region of a gene is used to drive expression of the operably linked nucleic acid. Optionally, a portion of the 5′ non-coding region of a gene containing a minimal amount of the 5′ non-coding region needed to drive expression of the operably linked nucleic acid is used. Assays to determine the ability of a designated portion of the 5′ non-coding region of a gene to drive expression of the operably linked nucleic acid are well-known in the art.

Particular promoters used to drive expression of NeuroD1 according to methods described herein are “ubiquitous” or “constitutive” promoters, that drive expression in many, most, or all cell types of an organism into which the expression vector is transferred. Non-limiting examples of ubiquitous promoters that can be used in expression of NeuroD1 are cytomegalovirus promoter; simian virus 40 (SV40) early promoter; rous sarcoma virus promoter; adenovirus major late promoter; beta actin promoter; glyceraldehyde 3-phosphate dehydrogenase; glucose-regulated protein 78 promoter; glucose-regulated protein 94 promoter; heat shock protein 70 promoter; beta-kinesin promoter; ROSA promoter; ubiquitin B promoter; eukaryotic initiation factor 4A1 promoter and elongation Factor I promoter; all of which are well-known in the art and which can be isolated from primary sources using routine methodology or obtained from commercial sources. Promoters can be derived entirely from a single gene or can be chimeric, having portions derived from more than one gene.

Combinations of regulatory sequences may be included in an expression vector and used to drive expression of NeuroD1. A non-limiting example included in an expression vector to drive expression of NeuroD1 is the CAG promoter which combines the cytomegalovirus CMV early enhancer element and chicken beta-actin promoter.

Particular promoters used to drive expression of NeuroD1 according to methods described herein are those that drive expression preferentially in glial cells, particularly astrocytes and/or NG2 cells. Such promoters are termed “astrocyte-specific” and/or “NG2 cell-specific” promoters.

Non-limiting examples of astrocyte-specific promoters are glial fibrillary acidic protein (GFAP) promoter and aldehyde dehydrogenase 1 family, member L1 (Aldh1L1) promoter. Human GFAP promoter is shown herein as SEQ ID NO:6. Mouse Aldh1L1 promoter is shown herein as SEQ ID NO:7.

A non-limiting example of an NG2 cell-specific promoter is the promoter of the chondroitin sulfate proteoglycan 4 gene, also known as neuron-glial antigen 2 (NG2). Human NG2 promoter is shown herein as SEQ ID NO:8.

Particular promoters used to drive expression of NeuroD1 according to methods described herein are those that drive expression preferentially in reactive glial cells, particularly reactive astrocytes and/or reactive NG2 cells. Such promoters are termed “reactive astrocyte-specific” and/or “reactive NG2 cell-specific” promoters.

A non-limiting example of a “reactive astrocyte-specific” promoter is the promoter of the lipocalin 2 (lcn2) gene. Mouse lcn2 promoter is shown herein as SEQ ID NO:5.

Homologues and variants of ubiquitous and cell type-specific promoters may be used in expressing NeuroD1.

In some cases, promoter homologues and promoter variants can be included in an expression vector for expressing NeuroD1. The terms “promoter homologue” and “promoter variant” refer to a promoter which has substantially similar functional properties to confer the desired type of expression, such as cell type-specific expression of NeuroD1 or ubiquitous expression of NeuroD1, on an operably linked nucleic acid encoding NeuroD1 compared to those disclosed herein. For example, a promoter homologue or variant has substantially similar functional properties to confer cell type-specific expression on an operably linked nucleic acid encoding NeuroD1 compared to GFAP, S100b, Aldh1L1, NG2, lcn2, and CAG promoters.

One of skill in the art will recognize that one or more nucleic acid mutations can be introduced without altering the functional properties of a given promoter. Mutations can be introduced using standard molecular biology techniques, such as site-directed mutagenesis and PCR-mediated mutagenesis, to produce promoter variants. As used herein, the term “promoter variant” refers to either an isolated naturally occurring or a recombinantly prepared variation of a reference promoter, such as, but not limited to, GFAP, S100b, Aldh1L1, NG2, lcn2, and pCAG promoters.

It is known in the art that promoters from other species are functional; e.g. the mouse Aldh1L1 promoter is functional in human cells. Homologues and homologous promoters from other species can be identified using bioinformatics tools known in the art, see for example, Xuan et al., Genome Biol., 6:R72 (2005); Zhao et al., Nucl. Acid Res., 33:D103-107 (2005); and Halees et al., Nucl. Acids. Res., 31:3554-3559 (2003).

Structurally, homologues and variants of cell type-specific promoters of NeuroD1 or and/or ubiquitous promoters have at least 80%, 81%, 82%, 83%, 84%, 85%, 86%, 87%, 88%, 89, 90%, 91%, 92%, 93%, 94%, 95%, 96%, 97%, 98%, 99%, or greater, nucleic acid sequence identity to the reference developmentally regulated and/or ubiquitous promoter and include a site for binding of RNA polymerase and, optionally, one or more binding sites for transcription factors.

A nucleic acid sequence which is substantially identical to SEQ ID NO:1 or SEQ ID NO:3 is characterized as having a complementary nucleic acid sequence capable of hybridizing to SEQ ID NO:1 or SEQ ID NO:3 under high stringency hybridization conditions.

In addition to one or more nucleic acids encoding NeuroD1, one or more nucleic acid sequences encoding additional proteins can be included in an expression vector. For example, such additional proteins include non-NeuroD1 proteins such as reporters, including, but not limited to, beta-galactosidase, green fluorescent protein, and antibiotic resistance reporters.

In particular embodiments, the recombinant expression vector encodes at least NeuroD1 of SEQ ID NO:2, a protein having at least 95% identity to SEQ ID NO:2, or a protein encoded by a nucleic acid sequence substantially identical to SEQ ID NO:1.

In particular embodiments, the recombinant expression vector encodes at least NeuroD1 of SEQ ID NO:4, a protein having at least 95% identity to SEQ ID NO:4, or a protein encoded by a nucleic acid sequence substantially identical to SEQ ID NO:2.

SEQ ID NO:9 is an example of a nucleic acid including a CAG promoter operably linked to a nucleic acid encoding NeuroD1, and further including a nucleic acid sequence encoding EGFP and an enhancer, WPRE. An IRES separates the nucleic acid encoding NeuroD1 and the nucleic acid encoding EGFP. SEQ ID NO:9 is inserted into an expression vector for expression of NeuroD1 and the reporter gene EGFP. Optionally, the IRES and nucleic acid encoding EGFP are removed, and the remaining CAG promoter and operably linked nucleic acid encoding NeuroD1 is inserted into an expression vector for expression of NeuroD1. The WPRE or another enhancer is optionally included.

Optionally, a reporter gene is included in a recombinant expression vector encoding NeuroD1. A reporter gene may be included to produce a peptide or protein that serves as a surrogate marker for expression of NeuroD1 from the recombinant expression vector. The term “reporter gene” as used herein refers to gene that is easily detectable when expressed, for example by chemiluminescence, fluorescence, colorimetric reactions, antibody binding, inducible markers, and/or ligand binding assays. Exemplary reporter genes include, but are not limited to, green fluorescent protein (GFP), enhanced green fluorescent protein (eGFP), yellow fluorescent protein (YFP), enhanced yellow fluorescent protein (eYFP), cyan fluorescent protein (CFP), enhanced cyan fluorescent protein (eCFP), blue fluorescent protein (BFP), enhanced blue fluorescent protein (eBFP), MmGFP (Zernicka-Goetz et al., Development, 124:1133-1137 (1997)), dsRed, luciferase, and beta-galactosidase (lacZ).

The process of introducing genetic material into a recipient host cell, such as for transient or stable expression of a desired protein encoded by the genetic material in the host cell is referred to as “transfection.” Transfection techniques are well-known in the art and include, but are not limited to, electroporation, particle accelerated transformation also known as “gene gun” technology, liposome-mediated transfection, calcium phosphate or calcium chloride co-precipitation-mediated transfection, DEAE-dextran-mediated transfection, microinjection, polyethylene glycol mediated transfection, heat shock mediated transfection, and virus-mediated transfection. As noted herein, virus-mediated transfection may be accomplished using a viral vector such as those derived from an adenovirus, an adeno-associated virus, and a lentivirus.

Optionally, a host cell is transfected ex-vivo and then re-introduced into a host organism. For example, cells or tissues may be removed from a subject, transfected with an expression vector encoding NeuroD1, and then returned to the subject.

Introduction of a recombinant expression vector including a nucleic acid encoding NeuroD1, or a functional fragment thereof, into a host glial cell in vitro or in vivo for expression of an exogenous NeuroD1 polypeptide in the host glial cell to convert the glial cell to a neuron is accomplished by any of various transfection methodologies.

Expression of exogenous nucleic acid encoding a NeuroD1 polypeptide (or a biologically active fragment thereof) in the host glial cell to convert the glial cell to a neuron is optionally achieved by introduction of mRNA encoding NeuroD1, or a functional fragment thereof, to the host glial cell in vitro or in vivo.

Expression of exogenous nucleic acid encoding a NeuroD1 polypeptide (or a biologically active fragment thereof) in the host glial cell to convert the glial cell to a neuron is optionally achieved by introduction of NeuroD1 protein to the host glial cell in vitro or in vivo. Details of these and other techniques are known in the art, for example, as described in J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; and Engelke, D. R., RNA Interference (RNAi): Nuts and Bolts of RNAi Technology, DNA Press LLC, Eagleville, P A, 2003.

An expression vector including a nucleic acid encoding NeuroD1 or a functional fragment thereof, mRNA encoding NeuroD1 or a functional fragment thereof, and/or NeuroD1 protein, full-length or a functional fragment thereof, is optionally associated with a carrier for introduction into a host cell in vitro or in vivo.

In particular aspects, the carrier is a particulate carrier such as lipid particles including liposomes, micelles, unilamellar or multilamellar vesicles; polymer particles such as hydrogel particles, polyglycolic acid particles or polylactic acid particles; inorganic particles such as calcium phosphate particles such as described in, for example, U.S. Pat. No. 5,648,097; and inorganic/organic particulate carriers such as described in, for example, U.S. Pat. No. 6,630,486.

A particulate carrier can be selected from among a lipid particle; a polymer particle; an inorganic particle; and an inorganic/organic particle. A mixture of particle types can also be included as a particulate pharmaceutically acceptable carrier.

A particulate carrier is typically formulated such that particles have an average particle size in the range of about 1 nm-10 microns. In particular aspects, a particulate carrier is formulated such that particles have an average particle size in the range of about 1 nm-100 nm.

Further description of liposomes and methods relating to their preparation and use may be found in Liposomes: A Practical Approach (The Practical Approach Series, 264), V. P. Torchilin and V. Weissig (Eds.), Oxford University Press; 2nd ed., 2003. Further aspects of nanoparticles are described in Moghimi et al., FASEB J., 19:311-30 (2005).

Expression of NeuroD1 using a recombinant expression vector is accomplished by introduction of the expression vector into a eukaryotic or prokaryotic host cell expression system such as an insect cell, mammalian cell, yeast cell, bacterial cell or any other single or multicellular organism recognized in the art. Host cells are optionally primary cells or immortalized derivative cells. Immortalized cells are those which can be maintained in vitro for at least 5 replication passages.

Host cells containing the recombinant expression vector are maintained under conditions wherein NeuroD1 is produced. Host cells may be cultured and maintained using known cell culture techniques such as described in Celis, Julio, ed., 1994, Cell Biology Laboratory Handbook, Academic Press, N.Y. Various culturing conditions for these cells, including media formulations with regard to specific nutrients, oxygen, tension, carbon dioxide, and reduced serum levels, can be selected and optimized by one of skill in the art.

In some cases, a recombinant expression vector including a nucleic acid encoding NeuroD1 is introduced into glial cells of a subject. Expression of exogenous nucleic acid encoding a NeuroD1 polypeptide (or a biologically active fragment thereof) in the glial cells “converts” the glial cells into neurons.

In some cases, a recombinant expression vector including a nucleic acid encoding NeuroD1 or a functional fragment thereof is introduced into astrocytes of a subject. Expression of exogenous nucleic acid encoding a NeuroD1 polypeptide (or a biologically active fragment thereof) in the glial cells “converts” the astrocytes into neurons.

In some cases, a recombinant expression vector including a nucleic acid encoding NeuroD1 or a functional fragment thereof is introduced into reactive astrocytes of a subject. Expression of exogenous nucleic acid encoding a NeuroD1 polypeptide (or a biologically active fragment thereof) or a functional fragment thereof in the reactive astrocytes “converts” the reactive astrocytes into neurons.

In some cases, a recombinant expression vector including a nucleic acid encoding NeuroD1 or a functional fragment thereof is introduced into NG2 cells of a subject. Expression of exogenous nucleic acid encoding a NeuroD1 polypeptide (or a biologically active fragment thereof) or a functional fragment thereof in the NG2 cells “converts” the NG2 cells into neurons.

Detection of expression of an exogenous NeuroD1 polypeptide (or a biologically active fragment thereof) following introduction of a recombinant expression vector including a nucleic acid encoding the exogenous NeuroD1 polypeptide or a functional fragment thereof is accomplished using any of various standard methodologies including, but not limited to, immunoassays to detect NeuroD1, nucleic acid assays to detect NeuroD1 nucleic acids, and detection of a reporter gene co-expressed with the exogenous nucleic acid encoding a NeuroD1 polypeptide (or a biologically active fragment thereof).

The terms “converts” and “converted” are used herein to describe the effect of expression of NeuroD1 or a functional fragment thereof resulting in a change of a glial cell, astrocyte, or reactive astrocyte phenotype to a neuronal phenotype. Similarly, the phrases “NeuroD1 converted neurons” and “converted neurons” are used herein to designate a cell including exogenous NeuroD1 protein or a functional fragment thereof which has a consequent neuronal phenotype.

The term “phenotype” refers to well-known detectable characteristics of the cells referred to herein. The neuronal phenotype can be, but is not limited to, one or more of: neuronal morphology, expression of one or more neuronal markers, electrophysiological characteristics of neurons, synapse formation, and release of neurotransmitter. For example, neuronal phenotype encompasses but is not limited to: characteristic morphological aspects of a neuron such as presence of dendrites, an axon, and dendritic spines; characteristic neuronal protein expression and distribution, such as presence of synaptic proteins in synaptic puncta and presence of MAP2 in dendrites; and characteristic electrophysiological signs such as spontaneous and evoked synaptic events.

In a further example, a glial phenotype such as an astrocyte phenotype and reactive astrocyte phenotype encompasses but is not limited to: characteristic morphological aspects of astrocytes and reactive astrocytes such as a generally “star-shaped” morphology; and characteristic astrocyte and reactive astrocyte protein expression, such as presence of glial fibrillary acidic protein (GFAP).

The term “nucleic acid” refers to RNA or DNA molecules having more than one nucleotide in any form including single-stranded, double-stranded, oligonucleotide, or polynucleotide. The term “nucleotide sequence” refers to the ordering of nucleotides in an oligonucleotide or polynucleotide in a single-stranded form of nucleic acid.

The term “NeuroD1 nucleic acid” refers to an isolated NeuroD1 nucleic acid molecule and encompasses isolated NeuroD1 nucleic acids having a sequence that has at least 70%, 75%, 80%, 85%, 90%, 91%, 92%, 93%, 94%, 95%, 96%, 97%, 98%, or 99% identity to the DNA sequence set forth in SEQ ID NO:1 or SEQ ID NO:3, or the complement thereof, or a fragment thereof, or an isolated DNA molecule having a sequence that hybridizes under high stringency hybridization conditions to the nucleic acid set forth as SEQ ID NO:1 or SEQ ID NO:3, a complement thereof or a fragment thereof.

The nucleic acid of SEQ ID NO:3 is an example of an isolated DNA molecule having a sequence that hybridizes under high stringency hybridization conditions to the nucleic acid set forth in SEQ ID NO:1. A fragment of a NeuroD1 nucleic acid is any fragment of a NeuroD1 nucleic acid that is operable in one or more aspects described herein including a NeuroD1 nucleic acid.

A nucleic acid probe or primer able to hybridize to a target NeuroD1 mRNA or cDNA can be used for detecting and/or quantifying mRNA or cDNA encoding a NeuroD1 protein. A nucleic acid probe can be an oligonucleotide of at least 10, 15, 30, 50, or 100 nucleotides in length and sufficient to specifically hybridize under stringent conditions to NeuroD1 mRNA or cDNA or complementary sequence thereof. A nucleic acid primer can be an oligonucleotide of at least 10, 15, or 20 nucleotides in length and sufficient to specifically hybridize under stringent conditions to the mRNA or cDNA, or complementary sequence thereof.

The terms “complement” and “complementary” refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10, or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids, and the stringency of hybridization conditions, as is well known in the art. The term “stringency of hybridization conditions” refers to conditions of temperature, ionic strength, and composition of a hybridization medium with respect to particular common additives such as formamide and Denhardt's solution.

Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

An example of high stringency hybridization conditions is hybridization of nucleic acids over about 100 nucleotides in length in a solution containing 6×SSC, 5×Denhardt's solution, 30% formamide, and 100 micrograms/mL denatured salmon sperm at 37° C. overnight followed by washing in a solution of 0.1×SSC and 0.1% SDS at 60° C. for 15 minutes. SSC is 0.15M NaCl/0.015M Na citrate. Denhardt's solution is 0.02% bovine serum albumin/0.02% FICOLL/0.02% polyvinylpyrrolidone. Under highly stringent conditions, SEQ ID NO:1 and SEQ ID NO:3 will hybridize to the complement of substantially identical targets and not to unrelated sequences.

Methods of treating a neurological condition in a subject in need thereof are provided which can include delivering a therapeutically effective amount of NeuroD1 to glial cells of the central nervous system or peripheral nervous system of the subject, the therapeutically effective amount of NeuroD1 in the glial cells results in a greater number of neurons in the subject compared to an untreated subject having the same neurological condition, whereby the neurological condition is treated.

The conversion of reactive glial cells into neurons also reduces neuroinflammation and neuroinhibitory factors associated with reactive glial cells, thereby making the glial scar tissue more permissive to neuronal growth so that neurological condition is alleviated.

The term “neurological condition” or “neurological disorder” as used herein refers to any condition of the central nervous system of a subject which is alleviated, ameliorated, or prevented by additional neurons. Injuries or diseases which result in loss or inhibition of neurons and/or loss or inhibition of neuronal function are neurological conditions for treatment by methods described herein.

Injuries or diseases which result in loss or inhibition of glutamatergic neurons and/or loss or inhibition of glutaminergic neuronal functions are neurological conditions that can be treated as described herein. Loss or inhibition of other types of neurons, such as GABAergic, cholinergic, dopaminergic, norepinephrinergic, or serotonergic neurons can be treated with the similar method.

The term “therapeutically effective amount” as used herein is intended to mean an amount of an inventive composition which is effective to alleviate, ameliorate, or prevent a symptom or sign of a neurological condition to be treated. In particular embodiments, a therapeutically effective amount is an amount which has a beneficial effect in a subject having signs and/or symptoms of a neurological condition.

The terms “treat,” “treatment,” “treating,” and “NeuroD1 treatment” or grammatical equivalents as used herein refer to alleviating, inhibiting, or ameliorating a neurological condition, symptoms or signs of a neurological condition, and preventing symptoms or signs of a neurological condition, and include, but are not limited to, therapeutic and/or prophylactic treatments.

Signs and symptoms of neurological conditions are well-known in the art along with methods of detection and assessment of such signs and symptoms.

In some cases, combinations of therapies for a neurological condition of a subject can be administered.

According to particular aspects an additional pharmaceutical agent or therapeutic treatment administered to a subject to treats the effects of disruption of normal blood flow in the CNS in an individual subject in need thereof include treatments such as, but not limited to, removing a blood clot, promoting blood flow, administration of one or more anti-inflammation agents, administration of one or more anti-oxidant agents, and administration of one or more agents effective to reduce excitotoxicity

The term “subject” refers to humans and also to non-human mammals such as, but not limited to, non-human primates, cats, dogs, sheep, goats, horses, cows, pigs and rodents, such as but not limited to, mice and rats; as well as to non-mammalian animals such as, but not limited to, birds, poultry, reptiles, and amphibians.

Embodiments of inventive compositions and methods are illustrated in the following examples. These examples are provided for illustrative purposes and are not considered limitations on the scope of inventive compositions and methods.

### Examples

### Materials and Methods

### Experimental Animals

The 5×FAD mice have three mutations on human APP [Swedish (K670 N/M671 L), London (V717I) and Florida (I716V)] and two mutations on human PS1 proteins (M146L and L286V). These 5×FAD transgenic mice recapitulate the major features of AD including amyloid pathology, neurodegeneration, and learning and memory impairments and therefore serve as a classic AD animal model (Oakley et al., J. Neurosci., 26:10129-10140 (2006)).

### Virus Vector Construct and Production

To specifically target GFAP-expressing cells, the Cre-recombinase-dependent AAV vectors were constructed. The conversion factors were inserted in an antisense direction in these flip-excision (FLEX) vectors, flanked by two pairs of antiparallel, heterotypic loxP sites (Atasoy et al., J. Neurosci., 28:7025-7030 (2008)). The Cre gene was placed under the promoter of glial fibrillary acidic protein (GFAP), and the conversion factor or GFP control gene was placed under CAG promoter on each individual vectors. Firstly, the hGFAP promoter from pDRIVE-hGFAP plasmid (InvivoGen, inc) was inserted to replace CMV promoter in the pAAV-MCS (Cell Biolab) between Mlul and Sacll site. PCR was applied to clone the Cre gene from hGFAP-Cre (Addgene, plasmid #40591, gift of Dr. Albee Messing) and then the Cre was inserted into pAAV MCS between EcoRI and Sal1 sites for the generation of pAAV-hGFAP::Cre vector. The cDNA-encoding NeuroD1, GFP was cloned by PCR from the retrovirual constructs described in the previous work (Guo et al., Cell Stem Cell, 14:188-202 (2014)) to construct the pAAV-FLEX-GFP and pAAV-FLEX-NeuroD1-P2A-GFP vectors. The NeuroD1 gene was fused with P2A-GFP and further subcloned and inserted between the Kpn1 and Xho1 sites of the pAAV-FLEX-GFP vector (Addgene plasmid #28304, gift from Dr. Edward Boyden). All plasmid constructs were sequenced for verification. 293AAV cells (Cell Biolabs) were cultured for the production of recombinant AAV9. That is, the 293AAV9 was transfected with the triple plasmids: pAAV expression vector, pAAV9-RC (Cell Biolab), and pHelper (Cell Biolab). After 72 hours of transfection, cells were harvested from the cultured medium and centrifuged, followed by 4 times of freezing and thawing by keeping it in dry ice/ethanol or 37° C. water bath alternately. Next, the raw AAV lysate was purified by centrifugation at 54,000 rpm for 1 hour in discontinuous iodixanol gradients with a Beckman SW55Ti rotor. After centrifugation, the supernatant containing the virus were filtered by the Millipore Amicon Ultra Centrifugal Filters to obtain the pure virus. The virus titer for hGFAP::Cre was 1.2×10¹² GC/mL, 1.4×10¹² GC/mL for CAG::FLEX-NeuroD1-P2A-GFP and 1.4×10¹² GC/mL for CAG::FLEX-GFP, was measured by QuickTiter™ AAV Quantitation Kit (Cell Biolabs).

Stereotaxic Intracranial Viral injection

Surgeries were performed on mice for intracranial injection of virus. Mice were anesthetized by intraperitoneally injection with 2.5% Avetin (10 mL/kg) and then placed in a stereotaxic setup. Artificial eye ointment was then applied for the cover and protection of the eyes. The mice were operated upon with a 49 middle-line scalp incision and a drilling hole on the skulls above the frontal cortex region. Direct intracranial injection (via a 5 μL syringe and a 34G needle) of virus were applied on each injection site (AP: +1.3 mm, ML: 1.4 mm, DV: −1.0 mm). For each site, the total injection volume was 2 μL; and the flow rate was controlled at 0.2 μL/minute. The needle was kept in place for another 10 minutes after injection, and was then slowly withdrawn from the site.

### Immunohistochemistry and Quantification

Mice were anesthetized by being injected with 10 mL/kg 2.5% Avertin into the peritoneum, and then were perfused with ice-cold artificial cerebrospinal fluid (ACSF) to wash off blood in the brain, followed by 4% paraformaldehyde (PFA) fixation in phosphate-buffered saline (PBS, pH=7.4). The brain tissue was dissected and then fixed overnight at 4° C. with 4% PFA. Samples were further sliced in the sagittal direction at 45 μm by Leica vibratome and stored in 0.1 M PB at 4° C. For immunostaining, brain slice samples were rinsed with PBS for three times, 10 minute each time, and then incubated for 2 hours with blocking solution (a mixture of 0.3% Triton-X, 5% normal donkey serum, 5% normal goat serum in 0.1 M PBS) at room temperature. The brain slices were further incubated with the following primary antibodies at 4° C. overnight [in 5% normal donkey serum (NDS) and normal goat serum (NGS) in 0.1 M PBS]: Monoclonal anti-GAD67 (mouse, 1:500, Millipore, MAB5406); polyclonal anti-GABA (rabbit, 1:1000, Sigma, A2052); Monoclonal anti-Parvalbumin (mouse, 1:2000, Sigma, P3088); Rabbit monoclonal anti-β amyloid 1-42 (rabbit, 1:2000, Invitrogen, 700254); Polyclonal anti-Glial Fibrillary Acidic Protein (chicken, 1:1000, Millipore, AB5541); polyclonal anti-Iba1 (rabbit, 1:500, Wako, 019-19741); monoclonal anti-iNOS (mouse, 1:500, BD, 610328); polyclonal anti-GFP (chicken, 1:1000, Abcam, ab13970); monoclonal anti-NeuroD1 (mouse, 1:500, Abcam, ab60704); polyclonal anti-IL1 f3 (rabbit, 1:1000, Abcam, Ab9722); polyclonal anti-MAP2 (chicken, 1:500, Abcam, ab5392); monoclonal anti-synaptophysin (mouse, 1:500, Millipore, MAB368); polyclonal anti-vesicular glutamate transporter 1 (guinea pig, 1:3000, Millipore, ab5905); monoclonal anti-neurofilament 200 (mouse, 1:500, Sigma, NO142); monoclonal anti-Lytic (Rat, 1:1000, Abcam, ab15627); polyclonal anti-AQP4 (rabbit, 1:1000, Santa Cruz, sc-20812); polyclonal anti-doublecortin (goat, 1:500, Santa Cruz, sc-8066); anti-nestin (mouse, 1:500, Neuromics, M015056); and polyclonal anti-Ki67 (rabbit, 1:1000, Abcam, ab15580). After washing the samples three times with PBS, the brain sections were then incubated with the appropriate secondary antibodies conjugated to Alexa Fluor 488 (1:1000, Jackson ImmunoResearch) or Alexa Fluor 647 (1:1000, Jackson ImmunoResearch) for 2 hours at room temperature. After washing the slices with PBS for 3 times, the brain sections were mounted on slides with the anti-fading mountant with DAPI (Invitrogen by Thermo Fisher Scientific, P36931). For Thioflavin-s staining, common procedures were performed on the brain samples according to the immunohistochemistry protocol described above. After the tissue sections were incubated in secondary antibody, the samples were firstly washed by diluted Thioflavin-s in PBS (2 μg/mL) for 10 minutes on the shaker. Next, wash the brain samples using PBS for twice, 10 minutes for each time. Samples were then mounted with ProLong Gold Antifade Mountant (Life Technologies, P36934) on the slides. Fluorescent images were acquired with a Keyence microscope (BIOREVO BZ9000 viewer & analyzer), an Olympus confocal microscope (FV 1000), or Zeiss confocal microscope (image acquired and processed by ZEISS ZEN microscope software). The confocal acquisition parameter setting remained the same for each individual target protein immunostaining. In chapter 2, for each sample, images were taken at 3 random locations at similar cortical layer within the 1000 μm of the injection core (where many converted neurons were observed) were taken. The quantitative data were averaged then to represent the sample. The images were further analyzed by the ImageJ software at the same settings (ImageJ 1.46r, Wayne Rasband, National Institutes of Health, USA) and graphed by GraphPad Prism 6.

### Human Aβ Elisa

The frontal cortex regions from the brains of AD+GG− and AD+GG+ littermates were isolated and lysed with NP40 cell lysis buffer (Invitrogen, FNN0021) with 1 mM PMSF protease inhibitor (Thermo Scientific, 36978) and protease inhibitor cocktail (Sigma-Aldrich, P2714), followed by centrifugation at 13,000 rpm at 4° C. Supernatant was collected for the quantitative analysis of Aβ load via ELISA test. Human Aβ42 ELISA kit (Invitrogen, KHB3441) and human Aβ40 ELISA kit (Invitrogen, KHB3481) were applied for the measurement of Aβ level. Protocol of the ELISA kits was strictly followed: First, standards and samples were loaded to the corresponding wells in the plate coated with the Aβ antibody (included in the ELISA kit) for the purpose of antigen binding. Second, human Aβ42 detector antibody (or human Aβ40 antibody, correspondingly) were added in the wells, tapped the plate to mix the solution thoroughly, followed by an incubation of 3 hours on a shaker at room temperature. Then, discard the solution and wash the wells with 1× wash buffer for 4 times. The HRP-conjugate antibody was incubated with the samples for 30 minutes at room temperature, followed by washing the wells with 1× wash buffer for 4 times. Then, stabilized chromogen was applied in each well for another incubation of 30 minutes in a dark place. After treating the sample in each well with the stop solution, read the plate within 30 minutes to obtain the absorbance at 450 nm and generate the standard curve (SpectraMax Plus 384 Microplate Reader). The Aβ42 and Aβ40 level of the unknown samples would be analyzed and quantified with the value of optical density at 450 nm and the known concentration of the standard ladder. Each sample was repeated twice, and the average value was analyzed.

### Brain Slice Electrophysiology

About 1 month after injection, brain cortical region was dissected and sliced with a Leica vibratome at 300 μm for brain slice recording. Cold cutting solution (in mM) contained 75 sucrose, 87 NaCl, 2.5 KCl, 0.5 CaCl₂, 7 MgCl₂, 25 NaHCO₃, 1.25 NaH₂PO₄, and 20 glucose. Brain slices were maintained in artificial cerebral spinal fluid (ACSF) (in mM) containing 119 NaCl, 2.5 KCl, 1.25 NaH₂PO₄, 26 NAHCO₃, 1.3 MgCl₂, 2.5 CaCl₂, 10 glucose, and bubbled with 95% 02 and 5% CO₂. The brain slices were incubated in ACSF and then perform the whole-cell recordings on these samples, with a pipette solution containing (in mM) 135 K-Gluconate, 10 KCl, 5 Na-phosphocreatine, 10 HEPES, EGTA, MgATP, and 0.5 Na₂GTP (pH 7.3, adjusted with KOH, 290 mOsm/L). Set pipette resistance at 3-5 MΩ, series resistance at 20-40 MΩ, holding potential for voltage-clamp at −70 mV. pClamp 9 software (Molecular Devices, Palo Alto, Calif.) was utilized for data collection and Clampfit, and Synaptosoft software was used for analysis. The adult brain slice were prepared following the former protocols (Ting et al., Methods Mol. Biol., 1183:221-242 (2014)). In brief, the 12-14 months old adult transgenic mice were transcardially perfused with cutting solution (in mM): 93 NMDG, 93 HCl, 30 NaHCO₃, 20 HEPES, 15 Glucose, 12 N-Acetyl-L-cysteine, 7 MgSO₄, 2.5 KCl, 1.25 NaH₂PO₄, 5 Sodium ascorbate, 3 Sodium pyruvate, 2 Thiourea, 0.5 CaCl₂, pH range 7.3-7.4, 300 mOsmo, bubbled with 95% 02/5% CO₂. The mouse brain was further dissected and cut at 300 μm in the cutting solution, and incubated at room temperature. Brain slices were collected in the cutting solution and incubated for 12-15 minutes at 32-34° C. The slices were kept in the holding solution with continuous 95% 02/5% CO₂ bubbling (in mM): 92 NaCl, 30 NaHCO₃, 20 HEPES, 15 Glucose, 12 N-Acetyl-L-cysteine, 2.5 KCl, 1.25 NaH₂PO₄, 5 Sodium ascorbate, 2 Thiourea, 3 Sodium pyruvate, 2 MgSO₄, and 2 CaCl₂. After recovery of the sample in the holding solution for 0.5 hours, patch-clamp recording was conducted in the standard aCSF (in mM): 124 NaCl, 26 NaHCO₃, 10 Glucose, 2.5 KCl, 1.25 NaH₂PO₄, 1.3 MgSO₄, and 2.5 CaCl₂.

cDNA synthesis and quantitative Real-time PCR

Quanta Biosciences qScript™ cDNA SuperMix was used for cDNA synthesis. To synthesize the cDNA, 1 μg RNA was used in total reaction volume of 20 μL for each sample. The program setting for synthesizing the cDNA was: 25° C. for 5 minutes, 42° C. for 30 minutes, 85° C. for 5 minutes, and held at 4° C. The cDNA product was further diluted five times using the RNase/DNase-free H₂O. Primers used for the quantitative Real-time PCR were designed by the Applied Biosystems Primer Express software. Reagent including Quanta Biosciences PerfeCTa™ SYBR® Green SuperMix, ROX™ was used for this experiment. 5 μL cDNA corresponding to 1 μg of total RNA was used in the total reaction volume of 25 μL. The program parameters were: 40 PCR cycles of 95° C. for 15 seconds and 65° C. for 45 seconds for amplification. After the PCR cycles, the melt curve was checked, and the comparative Ct value for each target gene was measured. GAPDH was used as the internal control gene, and relative gene expression was analyzed with respect to the gene expression in DMSO treated control group. Quantitative real-time PCR data had two replicates of PCR reaction for each sample.

### Data Analysis

Data were represented as mean±s.e.m. Student's t-test was used for statistical analysis in two-group comparison. One-way ANOVA or two-way ANOVA analysis was applied for comparison among multiple groups. Statistical significance was set at p<0.05. In the olfactory behavior test, for the calculation of F and P value of cross-habituation index, the data was analyzed using two-way ANOVA with LSD post hoc test via SPSS software. Statistical significance was set at p<0.05, labeled as *. p<0.01, labeled as **. p<0.001, labeled as ***. All behavioral tests and analyses were performed blindly.

### Example 1—Beneficial Effects of NeuroD1-Mediated Astrocyte-to-Neuron Conversion in an Alzheimer's Disease Mouse Model

NeuroD1 Over-Expression in Reactive Glia Enables the Astrocyte-to-Neuron Conversion with High Efficiency in 5×FAD Mouse Brain

In the AD brain, typical hallmarks include gliosis, neuronal loss, amyloid plaques, and intracellular neurofibrillary tangles. Gliosis has been reported to be highly enhanced in human AD cortices (Castillo et al., Scientific Reports, 7:17762 (2017)) and AD transgenic mouse models such as 5×FAD mice and Tg2576 AD mice (Games et al., Nature, 373:523-527 (1995); Nussbaum et al., Nature, 485:651-655 (2012); and Oakley et al., J. Neurosci., 26:10129-10140 (2006)). Particularly, the amyloid deposition and gliosis in 5×FAD mouse brains begins at 2 months of age, and is largely accumulated at deeper cortical layers and subiculum regions. Additionally, the neuron number decreases with age in 5×FAD brain during the pathological progression (Oakley et al., J. Neurosci., 26:10129-10140 (2006)). The abnormally increased astrocyte number and the reduced neuronal number results in an imbalanced ratio between neurons and astrocytes inside the brain, which further accounts for the dysfunction of the brain circuits. Therefore, we hypothesized that by direct in vivo conversion of reactive astrocytes to neurons, excessive reactive astrocytes will be reduced and utilized for replenishment of the lost neurons in 5×FAD brains.

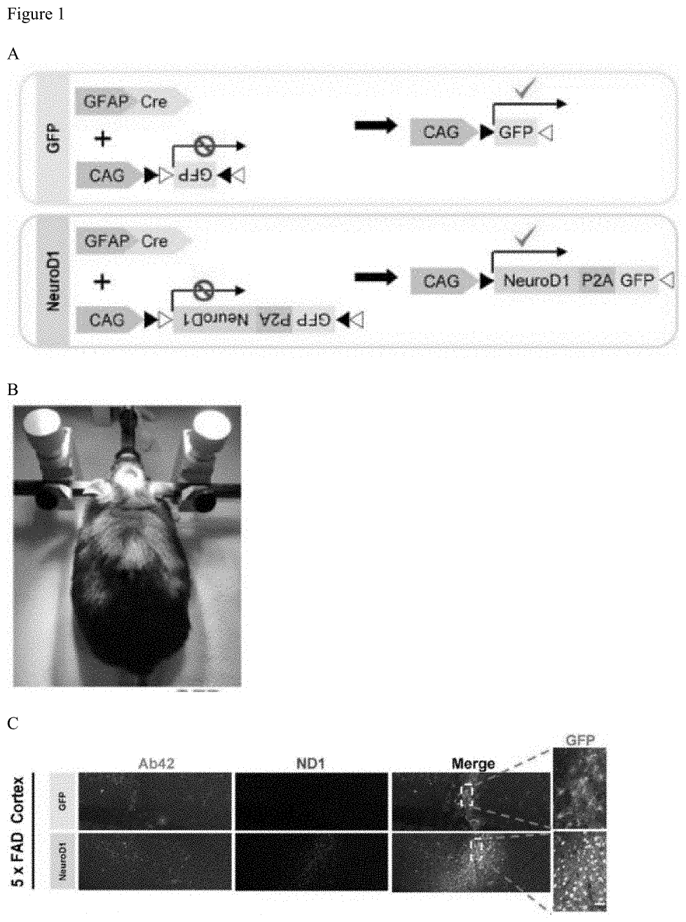

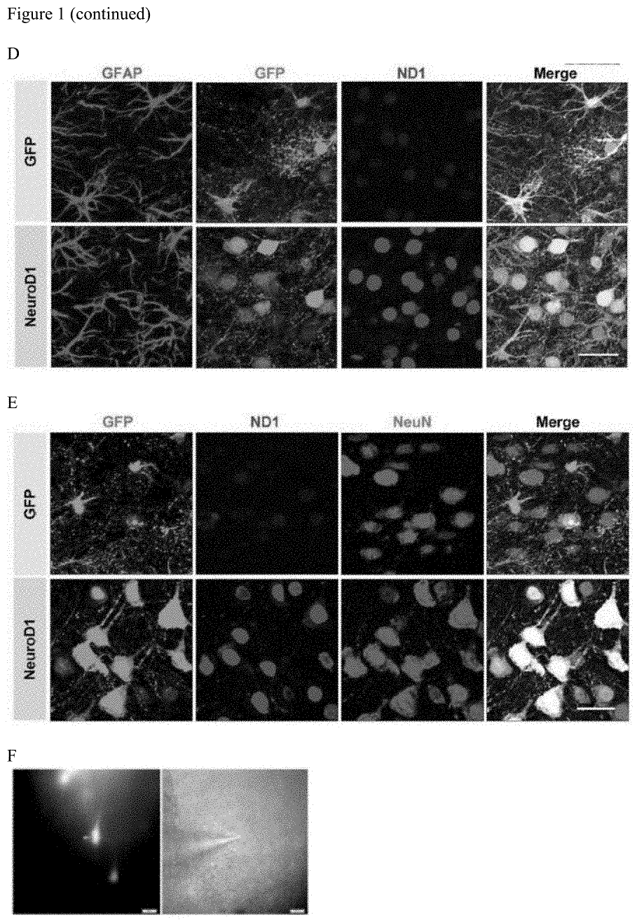

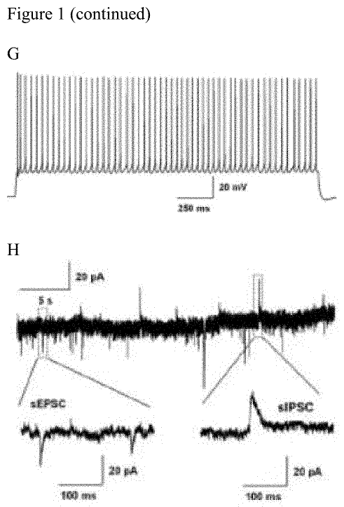

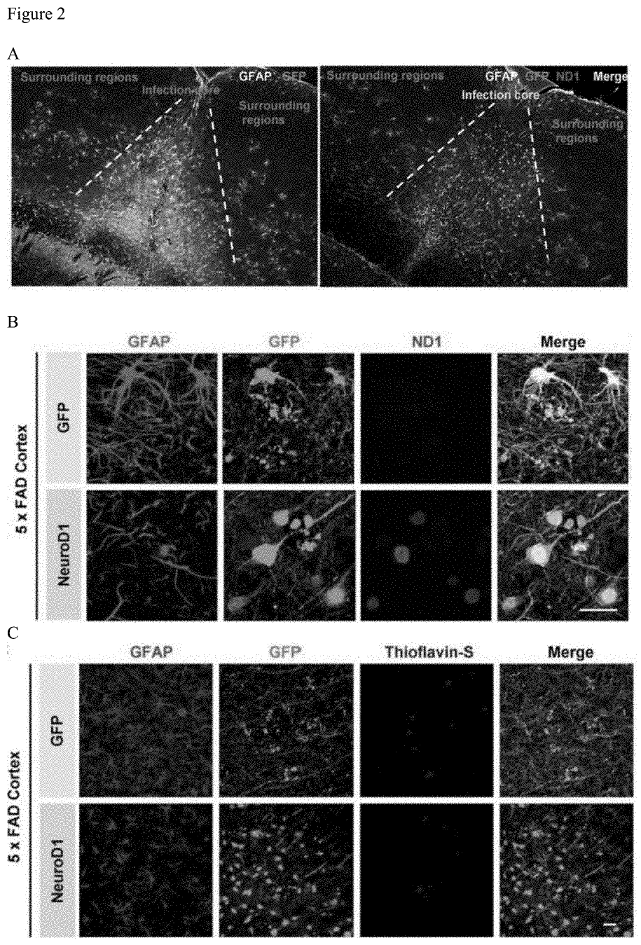

To achieve this goal, we designed and constructed the Adeno-associated virus serotype 9 (AAV9) vectors expressing Cre under the control of the reactive astrocyte GFAP promoter (AAV9-GFAP-Cre), together with an AAV9 vector expressing NeuroD1-GFP or GFP sham control under CAG promoter (AAV9-CAG-loxP-NeuroD1-P2A-GFP-loxP or AAV9-CAG-loxP-GFP-loxP, respectively). Therefore, in the GFP control group, only GFP will be over-expressed in the reactive astrocytes (GFAP+ cells), whereas NeuroD1 and GFP will be co-overexpressed in the reactive astrocytes in NeuroD1 group (FIG. 1A). Stereotaxic injection was conducted on the mouse brain cortical region (coordinate: L/R: ±1.4, A/P: +1.3, DV: −1.0 mm) (FIG. 1B). In this sub-region of cortex in 5×FAD, a heavy burden of amyloid plaques had already accumulated and astrocytes displayed the atypical morphology of expanded cell body with elongated and thicker processes, indicating the hyperactive state of astrocytes in such pathological condition. We next dissected the brain tissue sample and performed immunostaining to examine the NeuroD1 expression level in the injected region in 5×FAD mice brains at 30 days post-injection. Corroborated with our previous data (Guo et al., Cell Stem Cell, 14:188-202 (2014)), the reactive astrocytes have been efficiently converted into neuron-morphology like cells by NeuroD1 over-expression at 30 dpi. Notably, astrocytes with GFP over-expression remain the glia morphology in GFP control group (FIG. 1C). To further identify the infected cells and the converted cells, we conducted co-immunostaining of reactive astrocytes marker GFAP with GFP, mature neuronal marker NeuN (Neuronal Nuclei) with GFP, respectively. Representative images indicate our AAV9-GFAP-Cre system has high specificity in astrocytes based on the observation of the majority of infected cells are GFAP+(reactive astrocytes marker, magenta) (FIG. 1D). Interestingly, the infected reactive astrocytes that have NeuroD1 over-expression (which, when viewed in color, stained red) have already been successfully converted into mature neurons (NeuN+, which, when viewed in color, stained magenta) in the 5×FAD cortex by 30 dpi (FIG. 1E). Representative traces of action potential (FIG. 1G), sEPSC and sIPSC (FIG. 1H) were recorded in electrophysiological study on the converted neuron (bright field phase image, 25 dpi) (FIG. 1F). Taken together, the above data confirm that NeuroD1 over-expression enables the direct in vivo conversion from reactive astrocytes to mature and fully functional neurons with high efficiency in 5×FAD cortex within 1 month.

### NeuroD1-Mediated Astrocyte-to-Neuron Conversion Ameliorated the Hyperactive State of Astrocytes in 5×FAD Mouse Brain

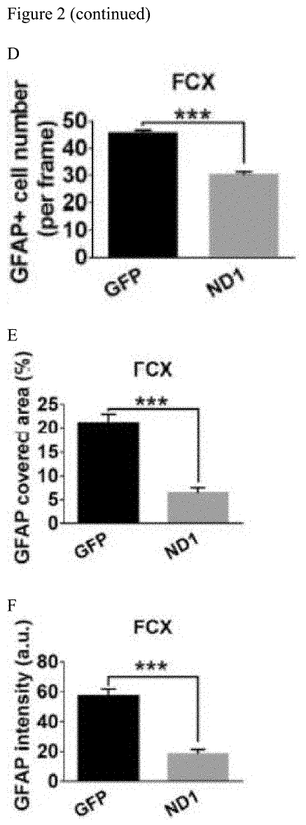

In brain, healthy astrocytes play a critical role in maintaining and regulating the normal neuronal communication, synaptic physiology as well as energy metabolism (Freeman, Science, 330:774-778 (2010)). However, in the pathological background of AD brain, abnormal hyperactive astrocytes and astrogliosis have been widely reported (Burda and Sofroniew, Neuron, 81:229-248 (2014)). These abnormal changes to the astrocytes also interfere with normal brain functions and signaling pathways, including changing the glutamate and GABA recycling, potassium buffering, and even cholinergic and calcium regulations (Osborn et al., Prog. Neurobiol., 144:121-141 (2016)). We hypothesized that the conversion of reactive astrocytes to functional neurons can benefit the brain by reducing the reactive astrocytes number and the hyperactive state of astrocyte in 5×FAD mice brains. Here, our study shows that NeuroD1 over-expression in reactive astrocytes can efficiently convert reactive astrocytes into functional mature neurons in 5×FAD brain. To investigate whether this break-through and cutting-edged technique can be applied as a potential therapeutic method for the AD, we further examine the beneficial effects after the in vivo direct cell conversion induced by NeuroD1. Strikingly, several beneficial effects on the reactive astrocytes near the injection core region were observed 60 days after injection (here, we defined the beneficial effects that can be observed at early time point (˜60 DPI) as short-term beneficial effects) (FIG. 2A and FIG. 2B). Firstly, the number of the reactive astrocytes was decreased in NeuroD1 group, because the excessive reactive astrocytes were converted into mature neurons by over-expression of NeuroD1 (FIGS. 2C and 2D: GFAP+ cell number in GFP control group: 45.5±1.0; GFAP+ cell number in NeuroD1 group: 30.3±1.1, N=8, p<0.001). Secondly, the remaining reactive astrocytes in NeuroD1 group displayed the morphology with less expanded cell body and thinner processes, indicated that the hyperactive state of reactive astrocytes was ameliorated when compared to the GFP control group (FIG. 2C-FIG. 2F: GFAP+ covered area percentage in GFP control group: 21.0±1.9; GFAP+ covered area percentage in NeuroD1 group: 6.4±1.1, N=8, p<0.001. GFAP+ intensity in GFP control group: 57.2±4.8; GFAP+ intensity in NeuroD1 group: 18.3±3.2, N=8, p<0.001).

### NeuroD1-Mediated Astrocyte-to-Neuron Conversion can Replenish the Neuron Pool in 5×FAD Mouse Brain

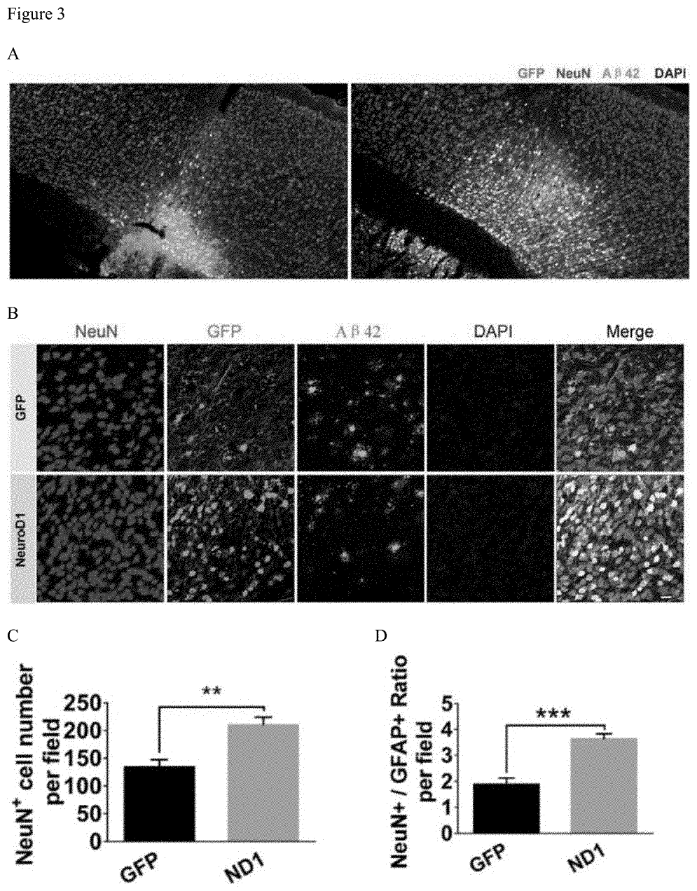

Along with the finding that excessive reactive astrocytes were dramatically reduced, we also observed a significantly increase in neurons in NeuroD1-mediated cell conversion group. Here, we carefully examined several different time points after NeuroD1 intervention (30 dpi, 60 dpi, 90 dpi, data not shown) and found that by 60 dpi a wide neuron induction can be observed. The 5×FAD mice initiated intracellular β-amyloid production as early as 1.5 months and started to develop extracellular amyloid plaques at 2 months old. By 6 months old, heavy burden of amyloid plaques were deposited throughout the cortex of 5×FAD mouse brain, leading to the gradual neuronal loss. The NeuroD1 group shows a significant increase of neuron numbers in the treated brain region when compared with GFP control group (FIG. 3B and FIG. 3C). Therefore, the abnormal neuron and astrocytes ratio in the pathological condition were reversed after NeuroD1 intervention (FIG. 3D). Our strategy replenishes the neuron pool by converting excess reactive astrocytes, which is another beneficial feature of NeuroD1-mediated astrocyte-to-neuron conversion.

### GABAergic Neurons can be Generated by NeuroD1-Mediated Astrocyte-to-Neuron Conversion in 5×FAD Cortex

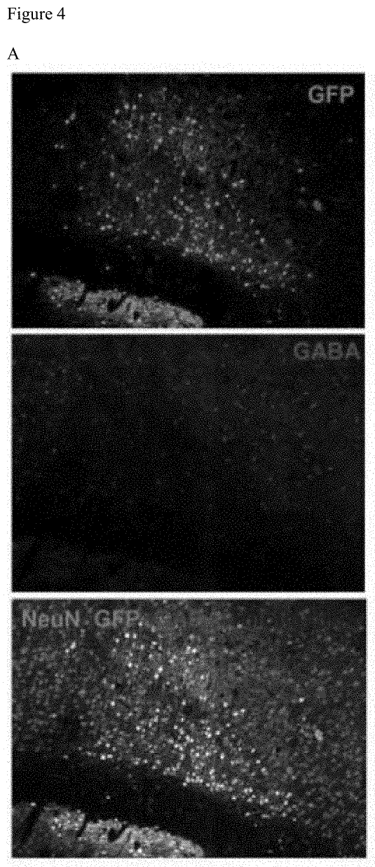

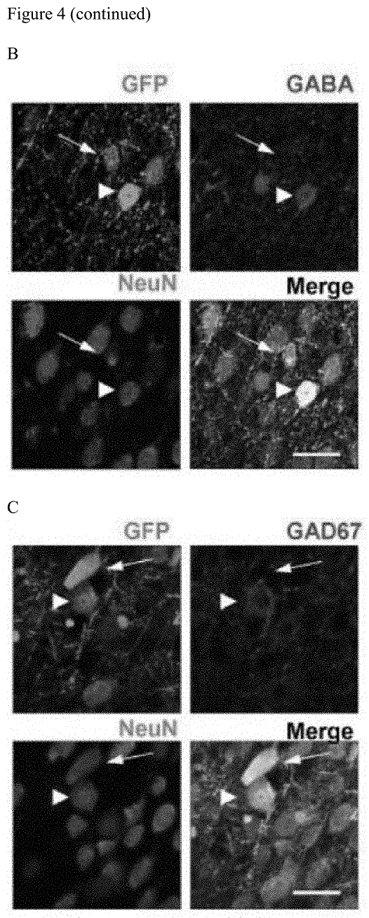

Multiple evidence has suggested that the GABAergic system is severely altered in the AD brain, including the GABAergic neuron loss (Schmid et al., Neuron, 92:114-125 (2016); and Verret et al., Cell, 149:708-721 (2012)) and changes of GABA synthesis (Limon et al., PNAS, 109:10071-10076 (2012)) and transport (Wu et al., Nature Comm., 5:4159 (2014)) during the AD pathogenesis. Therefore, we questioned whether our strategy could also regenerate adequate GABAergic neurons in the AD mouse brains. To address this concern, co-immunostaining of mature neuronal marker NeuN and GABAergic marker GABA was performed (FIG. 4A). Interestingly, besides the existing GABAergic neurons (FIG. 4B, arrow, GABA+ and NeuN+ cells), a group of converted neurons (FIG. 4B, arrow head, GFP+, GABA+ and NeuN+ cells) also express a similar level of GABA (indicated by GABA immunofluorescence) in the soma, comparing to the GABA level in the soma of remaining GABAergic neurons. To further validate that some of the converted neurons are GABAergic, we applied GABAergic marker GAD67 and found some converted neurons were GAD67 immunopositive. This observation indicates that NeuroD1 can convert astrocytes to GABAergic neurons in 5×FAD mouse cortex. Among the converted neurons, 18.5%±2.1% are GABA+ neurons, which is similar to the percentage of GABAergic neurons in total cortical neurons in non-pathological mouse brains. Hence, the NeuroD1-mediated astrocyte-to-neuron conversion can regenerate adequate numbers of GABAergic neurons in 5×FAD cortex regions to replenish the GABAergic neuron pool.

### NeuroD1 Treated 5×FAD has Less Abnormal Aggregates in the Brain

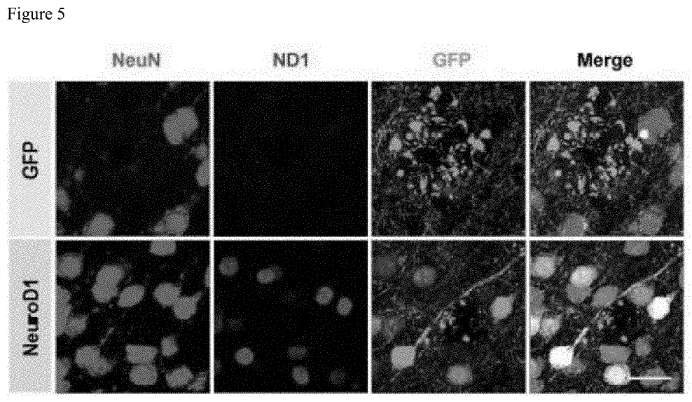

During the comparison of astrocytes and neuronal changes between the GFP control group and NeuroD1-mediated conversion group, we unexpectedly discovered a large amount of abnormal GFP-labeled large aggregates (which, when viewed in color, stained green) inside the GFP control group at 60 days after stereotaxic injection in 5×FAD mouse cortex (FIG. 5). However, this phenomenon was much reduced in NeuroD1 group. This novel finding suggests a healthier local environment in the NeuroD1 treated AD brains. The source of the abnormal GFP aggregates may be the debris of the GFP-infected cells in AD pathological condition, or other cells uptaking the GFP debris during the progression of AD pathology, which requires further identification.

Newly Converted Neurons have Less Intracellular Aβ Load in 5×FAD Cortex

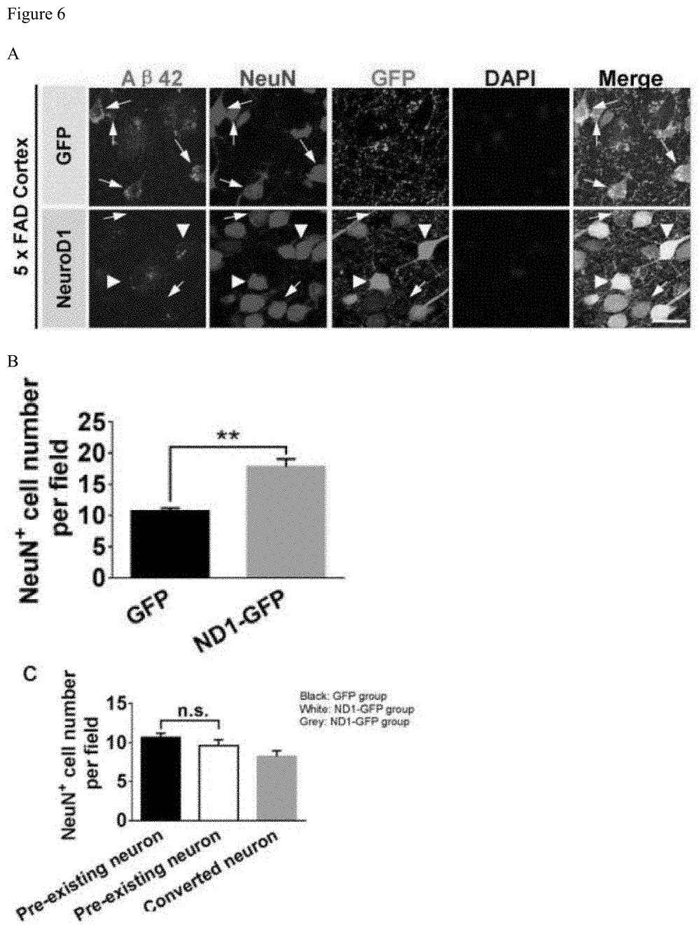

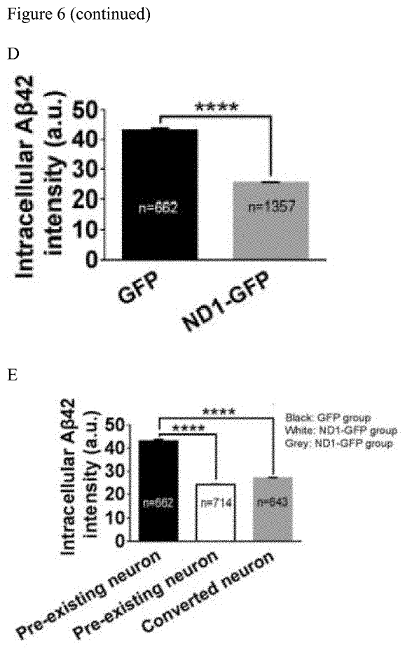

According to the interesting observations above, we further hypothesize that the cell conversion mediated by NeuroD1 may provide the brain with a healthier environment by reducing abnormal reactive astrocytes, replenishing neurons, and therefore rebuilding the normal communication between astrocytes and neuronal cells. Previous studies have also reported that reactive astrocytes (Gonzalez-Reyes et al., Front. Molec. Neurosci., 10:427 (2017)) and activated microglia (Venegas et al., Nature, 552:355-361 (2017)) play an important role in the progression of amyloid toxicity in many neurodegenerative diseases, including the AD, Huntington's disease, Parkinson's disease, amyotrophic lateral sclerosis, and multiple sclerosis (Liddelow et al., Nature, 541:481-487 (2017)). On one hand, the presence of amyloid interferes with the intracellular signaling pathways and normal functions of astrocytes. On the other hand, reactive astrocytes have increased levels of the three necessary components for Aβ production, including amyloid precursor protein, β-secretase (BACE1) and γ-secretase (Frost and Li, Open Biol., 7 (2017)). We next investigated whether our strategy of reprogramming the reactive astrocytes to neurons can impact the amyloid production and progression in 5×FAD mice brains. It is well recognized that Aβ is deposited extracellularly, there is growing evidence indicating that this peptide can also accumulated intraneuronally (LaFerla et al., Nature Rev. Neurosci., 8:499-509 (2007)), and may promote disease progression including synaptic dysfunction and neuron loss (Bayer and Wirths, Front. Aging Neurosci., 2:8 (2010)). To answer this question, we further examined the intracellular Aβ load by co-immunostaining of neuronal marker NeuN and β-amyloid (Aβ42). The intracellular Aβ level of all neurons in the infection core of each brain sample was carefully measured and analyzed. Surprisingly, only a minimum level of the intracellular Aβ42 is detected in the NeuroD1-converted new neurons, while the pre-existing neurons in the GFP control group contains large amount of intracellular Aβ42 (FIG. 6A and FIG. 6D, neuron number=662 from 3 5×FAD mice in GFP control group, neuron number=1357 from 3 5×FAD mice in NeuroD1 group, 60 DPI). Moreover, the intracellular Aβ42 level in the pre-existing neurons was also reduced in the NeuroD1 treated group (FIG. 6A and FIG. 6E: intracellular Aβ42 intensity in all pre-existing neurons in the infection core of GFP group: 43.1±0.7, analyzed pre-existing neuron number=662 from 3 5×FAD mice in GFP control group). Intracellular Aβ42 intensity in all pre-existing neurons in the infection core of NeuroD1 group: 24.2±0.1, analyzed pre-existing neuron number=714 from 3 5×FAD mice in NeuroD1 group. Intracellular Aβ42 intensity of all converted neurons in the infection core of NeuroD1 group: 27.0±0.2, analyzed converted neuron number=643 from 3 5×FAD mice in NeuroD1 group), suggesting that decreasing the reactive astrocytes can reverse or at least retard the β-amyloid pathological progression.

### NeuroD1-Mediated Astrocyte-to-Neuron Conversion Reduces the Pro-Inflammatory Microglia in 5×FAD Mouse Cortex

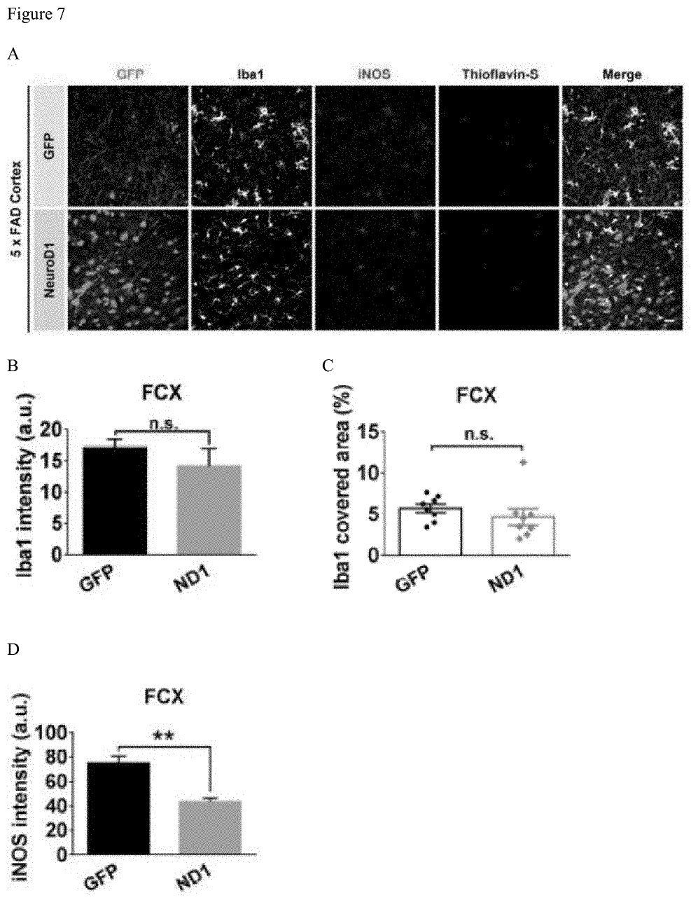

Besides the abundant amyloid deposits and the excessive reactive astrocytes, the Alzheimer's diseased brain is also characterized by an inflammatory response. This immune response is largely driven by the brain's intrinsic myeloid cells (microglia), which are closely involved in the pathological progression of AD. Amyloid β can prime the microglia by making them more susceptible to secondary stimulus and promotes their activation. Such priming effects on microglia results in a constant production of pro-inflammatory chemokines and cytokines via these cells, which further leads to the acceleration of pathogenesis and the exacerbation of the disease progression. In addition, these increased cytokines contribute to the maintenance of the reactive state of the primed microglia during the pathological progression of AD (Heppner et al., Nature Rev. Neurosci., 16:358-372 (2015)). Therefore, we first tested whether the NeuroD1-mediated direct cell conversion has any beneficial effects on the immune response in 5×FAD cortex by co-immunostaining the general microglia marker Iba1 (FIG. 7A, which, when viewed in color, stained grey) and the pro-inflammatory microglia subtype marker iNOS (FIG. 7A, which, when viewed in color, stained red). The microglia (FIG. 7A, Iba1+ cells, which, when viewed in color, stained grey) remain similar intensity in both groups, whereas the active state of microglia (activated microglia typically show abnormally expand cell body and thicker processes) in NeuroD1 group was slightly reduced, though no significant statistic differences can be concluded. The immunofluorescence of pro-inflammatory microglia subtype marker, iNOS, however, was largely reduced after NeuroD1-mediated astrocyte-to-neuron conversion, suggesting the pro-inflammatory response is mitigated under the NeuroD1 treatment in AD mouse brain.

Pro-Inflammatory Cytokine IL-1β was Down-Regulated after NeuroD1-Mediated Astrocyte-to-Neuron Conversion in 5×FAD Cortex

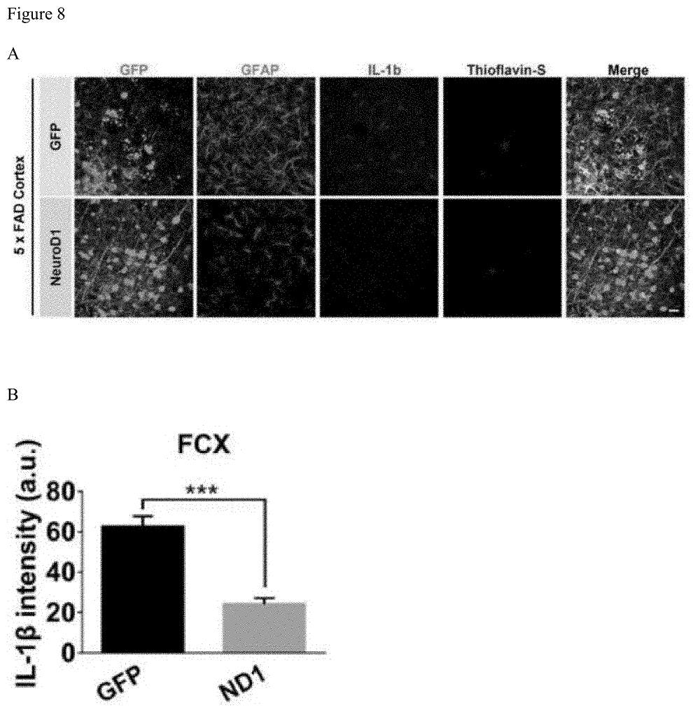

In recent years, accumulating in vitro (van Gijsel-Bonnello et al., PloS One, 12:e0175369 (2017)) and in vivo (Medeiros and LaFerla, Exp. Neurol., 239:133-138 (2013); Stamouli and Politis, Psychiatrike Psychiatriki, 27:264-275 (2016)) evidence supported the notion that increased levels of pro-inflammatory cytokines, including interleukin 1β (IL-1β), interleukin 6 (IL-6), interferon γ (IFN-γ), and tumor necrosis factor (TNF) play a role in the pathological progression of AD. Such increased levels of pro-inflammatory cytokines hampers the AD brain by suspending phagocytosis of amyloid beta (Stamouli and Politis, Psychiatrike Psychiatriki, 27:264-275 (2016)). Among the multiple pro-inflammatory cytokines, we were particularly interested in IL-1β, because interleukins are closely involved in the complex intercellular interactions among astrocytes, neurons and microglia in AD. Enhanced interleukin level impacts the efficacy of removal of amyloid plaques by microglia, and increases astrogliosis and neural death. In addition, interleukins regulate the intracellular signal transduction events that are necessary for the promotion of the inflammatory cascade characteristic of AD pathology (Stamouli and Politis, Psychiatrike Psychiatriki, 27:264-275 (2016)). To study the beneficial effects with respect to the pro-inflammatory response after NeuroD1-mediated conversion in 5×FAD mice brains, we examine the pro-inflammatory changes by conducting co-immunostaining of reactive astrocyte marker (GFAP) and interleukin-1β (IL-1β) at 60 days after injection. Surprisingly, in parallel with our previous finding that reactive astrocytes (GFAP+ cells, which, when viewed in color, stained magenta) were largely reduced (FIG. 8A and FIG. 8B), a remarkable decrease of interleukin-1β level (which, when viewed in color, stained red, IL-1β intensity in GFP control group: 62.7±5.0; IL-1β intensity in NeuroD1 group: 24.3±2.8) was also observed inside the reactive astrocytes after NeuroD1-mediated cell conversion in the treated region of 5×FAD mouse cortex. Taken together, the NeuroD1-mediated cell conversion benefits the AD brain by reducing the pro-inflammatory cytokine IL-1β, which may be the potential mechanism underlying the reduction of Aβ load in 5×FAD mouse brains.

NeuroD1 Converted Neurons Survived for More than 8 Months in the 5×FAD Mouse Brains

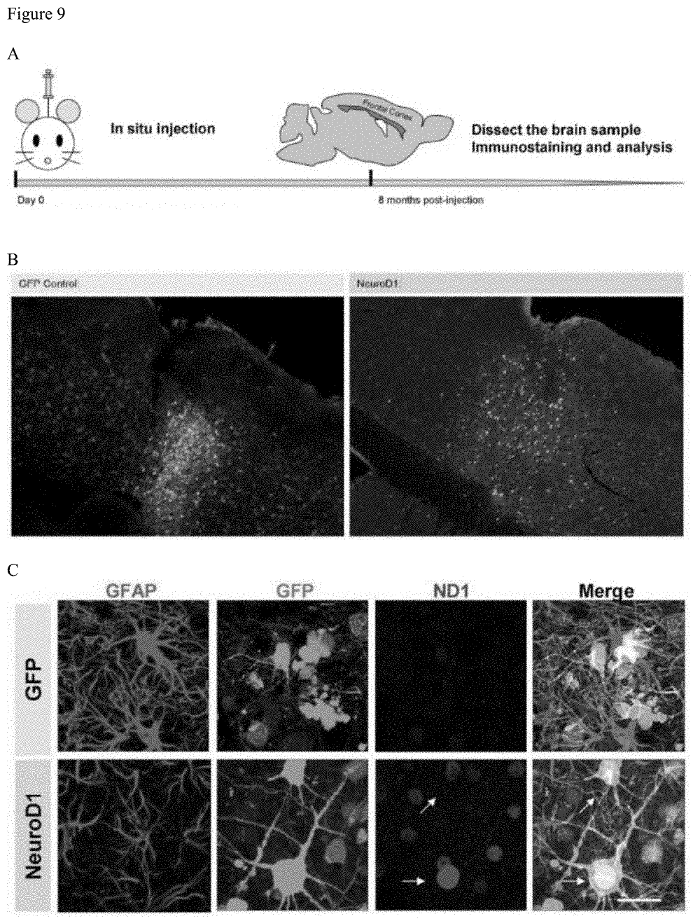

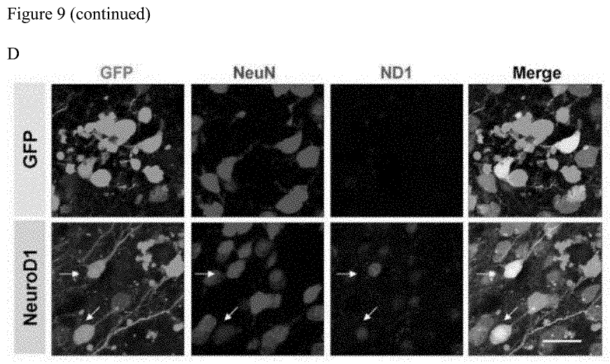

As described herein, NeuroD1 over-expression in reactive astrocytes can achieve efficient conversion to functional neurons, decrease intracellular Aβ load, and ameliorate the pro-inflammatory response in 5×FAD brain by 2 months after injection. We further questioned whether the converted neurons can survive in the 5×FAD mouse brains after a long term and what corresponding beneficial effects can be observed in the long-term. To answer the question, we applied the in situ delivery of either AAV9-GFAP-Cre mixed with AAV9-CAG-GFP or AAV9-GFAP-Cre mixed with 24 AAV9-CAG-NeuroD1-P2A-GFP into the cortex region (coordinate: L/R: ±1.4, A/P: 1.3, DV: −1.0) via stereotaxic injection. Brain samples were collected after ˜8 months for further investigation. The immunofluorescence analysis indicated that NeuroD1 converted neurons can still survive at 8 months after injection. The converted neurons possessed healthy neuronal morphology with multiple neurites (FIG. 9C and FIG. 9D, which, when viewed in color, stained green), and remained high expression level of NeuroD1 (FIG. 9C, which, when viewed in color, stained red) (FIG. 9D, which, when viewed in color, stained magenta). In contrast, large burden of GFP aggregates were observed in the GFP control group at 8 months post-injection. In this long-term treatment group, we suspected that the abnormal GFP aggregates came from two potential sources: (1) the debris of the dead infected astrocytes because of the constant accumulation of the Cre toxicity inside the cell; and/or (2) atrophy neurites of the leaked neurons in the GFP control group. Cre is supposed to only be over-expressed under the GFAP promoter (inside reactive astrocytes). However, during the long-term period after injection, Cre may be released into the vicinity regions from the dead astrocytes and consequently be uptaken by the surrounding neurons. Hence, some of the pre-existing neurons in GFP control group may also have GFP expression. However, they do not obtain NeuroD1 over-expression, supported by the immunostaining data (FIG. 9C and FIG. 9D). Since we are interested in the long-term beneficial effects of NeuroD1-mediated astrocyte-to-neuron conversion in AD pathological condition, we focused more on the examination and comparison of the neuronal and pathological markers changes instead of the subtle leaked labeling issue in this Cre-loxP system.

### Long-Term Effects of NeuroD1-Mediated Cell Conversion on Axons, Dendrites and Synapses in the 5×FAD Cortex

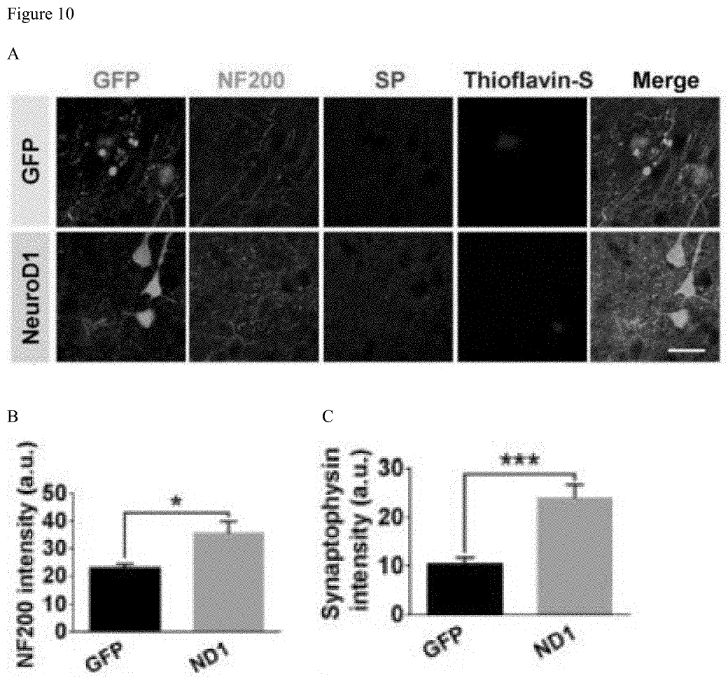

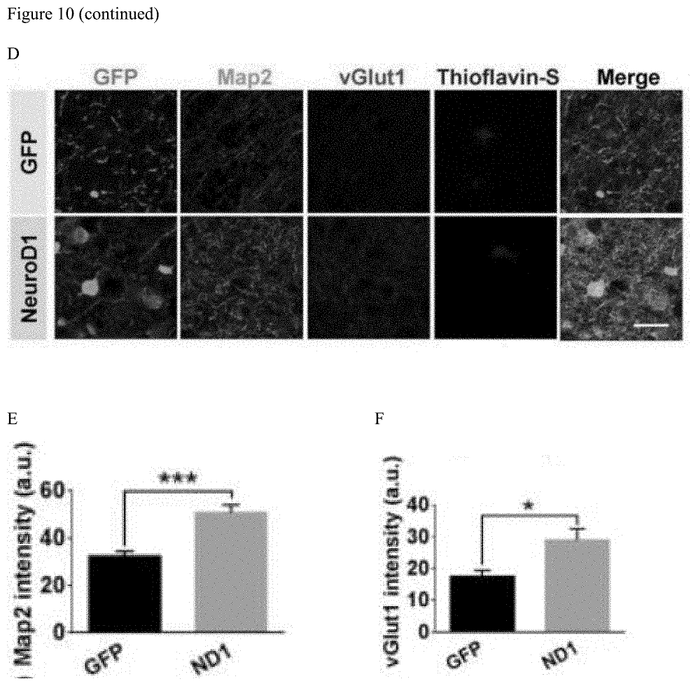

In the AD brain, synapses were venerable and gradually reduced during the pathological progression of AD. The loss of neuronal neurites and synapse leads to an impairment of learning and memory in AD and dementia (Mitew et al., Neurobiol. Aging, 34:2341-2351 (2013); and Palop and Mucke, Nat. Neurosci., 13:812-818 (2010)). Three major reasons may account for this symptom in AD. (1) Normal astrocytes promote the neuronal survival and outgrowth, facilitate the synapse formation and function and helps with the clearance of synaptic and myelin debris (Liddelow et al., Nature, 541:481-487 (2017)). However, the dysfunctional reactive astrocytes in AD background may interrupt this intercellular regulation and interfere with the synapse formation and maintenance. (2) Microglia, as most tissue macrophages, can support the CNS homeostasis and plasticity by protecting and remodeling synapses. Besides, the brain-derived neurotrophic factor (BDNF) synthesized by normal functional microglia is also critical for promoting the learning-related synapse formation (Prinz and Priller, Nature Rev. Neurosci., 15:300-312 (2014)). Previous work has reported that, the lack of neurotrophic factors such as BDNF may severely impair the neuronal integrity that resulted in synapse loss and disrupt synaptic functions (Parkhurst et al., Cell, 155:1596-1609 (2013)). (3) Other cascade molecules or proteins, for example, the C1q and C3 localized to synapses can regulate the synapse elimination (Hong et al., Science, 352:712-716 (2016)). However, this function is interrupted in AD condition. To study whether the NeuroD1-mediated conversion has long-term beneficial effects in maintaining adequate neuronal neurites and synapses in 5×FAD brain, here we examined the axon, dendrite, and synaptic density change by immunostaining at 8 months after the in vivo intervention. Particularly, for examination of the synaptic change, we tested synaptophysin as the global synaptic marker and vGluT1 as the excitatory synapse marker. Axons will be labeled by neurofilament 200 (NF200, FIG. 10A, which, when viewed in color, stained sapphire) whereas dendrites will be microtubule associated protein 2 (MAP2, FIG. 10B, which, when viewed in color, stained sapphire) immunopositive. Aβ aggregates were revealed by Thioflavin-s staining (FIG. 10A and FIG. 10B, which, when viewed in color, stained blue). A significant increase of the intensity of the global synaptic marker synaptophysin was observed (FIG. 10A, which, when viewed in color, stained red). In accordance with the increased intensity of synaptophysin, the excitatory synaptic marker vGluT1 (FIG. 10B, which, when viewed in color, stained red) also displayed a fairly enhanced intensity after NeuroD1 treatment. We also investigated the axons, dendrites, and synapse changes when the 5×FAD mice were 6 months old (2 months after injection). However, no significant differences were observed between GFP control group and NeuroD1 group. One possible reason accounting for that is the neurodegeneration and synaptic loss is not that severe when the 5×FAD mice are still in the early or middle-stage of AD pathological progression. These beneficial effects were magnified when 5×FAD mice were treated for a much longer time and examined at the late-stage of AD pathological progression when they develop severe symptoms including more neuronal and synaptic loss. Taken together, these data indicate that the NeuroD1-mediated cell conversion increases the synapse density by: (1) maintaining and preserving the synapses of the pre-existing neurons because of healthier brain environment, and (2) increasing new synapses when more new neurons are generated by conversion. NF200 and MAP2 intensity was also enhanced in NeuroD1 group, suggesting that axons and dendrites were both increased after NeuroD1 treatment. Therefore, our therapeutic strategy of NeuroD1-mediated astrocyte-to-neuron conversion plays a beneficial and supportive role in maintenance, protection, and generation of adequate axons, dendrites, and synapses for better sustenance of neuronal integrity in the brain, and may be served as a potential therapy for AD patients.

As demonstrated herein, the new neurons created using the methods and materials described herein can be neurons having properties to withstand the destructive CNS environment of a neurological condition (e.g., the destructive environment of an AD brain). In addition, the methods and materials described herein can be used to reestablish neuronal and/or astrocyte homeostasis within the brain of a mammal having a neurological conditions (e.g., AD).

### NeuroD1-Mediated Astrocyte-to-Neuron Conversion Protects the Blood Vessel Morphology in 5×FAD Mouse Cortex

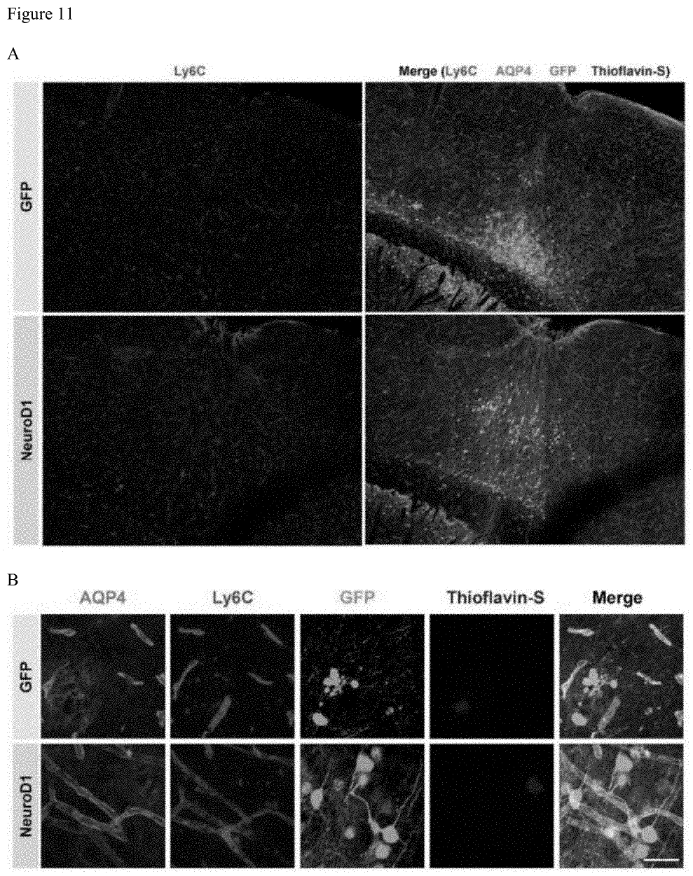

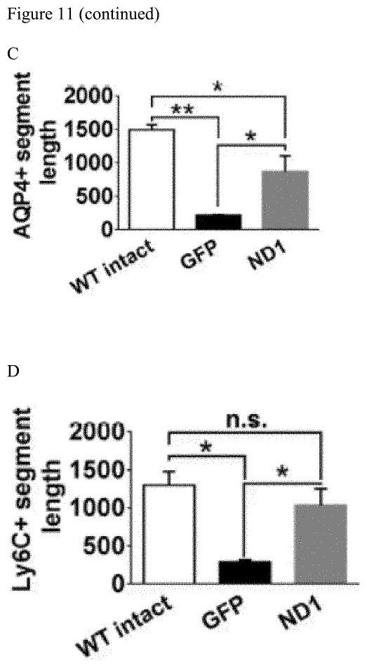

Early studies have elucidated that the pathogenesis of AD is characterized with accumulation and deposition of Aβ, neurodegeneration, activation of microglia and astrocyte, and importantly, the blood vessel regression and degeneration. The Aβ deposition in the blood vessel walls is also known as cerebral amyloid angiopathy (Jaunmuktane et al., Nature, 525:247-250 (2015)). The toxicity of accumulated amyloid-0 surround the blood vessels exhibit detrimental effects on the neurovascular unit and resulted in the vascular integrity and dysfunction through degeneration of endothelial cells and pericytes (Busch et al., Cell. Physiol. Biochem. Int. J. Exp. Cell. Physiol. Biochem. Pharmacol., 30:1436-1443 (2012)). Additionally, another study has reported that normal astrocytes, together with vascular smooth muscle cells and pericytes, are critical players in the modulation of vessel diameters, blood flow, and neurovascular plasticity (Kimbrough et al., J. Neurol., 138:3716-3733 (2015)). Therefore, in the AD brain, the hypertrophic reactive astrocytes and accumulation of amyloid plaques surround the blood vessels circumference and the oxidative-induced inflammation leads to the blood vessel morphological disruption and poor vascular responses (Marchesi, FASEB J., 25:5-13 (2011)). To investigate whether reduction of reactive astrocytes by NeuroD1-mediated astrocyte-to-neuron conversion may alleviate the hamper on blood vessel integrity, we examined blood vessel integrity by co-immunostaining of blood vessel marker Lymphocyte antigen 6 complex (Ly6C) and reactive astrocyte end-feet marker Aquaporin 4 (AQP4) on the 5×FAD brain samples at 8 months after NeuroD1 or GFP control intervention (FIG. 11). Surprisingly, in contrary to the shorter Ly6c+ and AQP4+ segments in the GFP control group, the NeuroD1 group displayed much longer Ly6c+ and AQP4+ segments, suggesting the integrity of blood vessels is preserved better in the NeuroD1 group (FIG. 11). Taken together, the results provided herein demonstrate the protective role of NeuroD1-mediated cell conversion in the blood vessel integrity. Wild-type intact mouse brains were also examined as controls.

### Example 2—Global Infection of AD Mouse Brain by Multiple Intracranial Injections of NeuroD1 AAV-PHP.eB

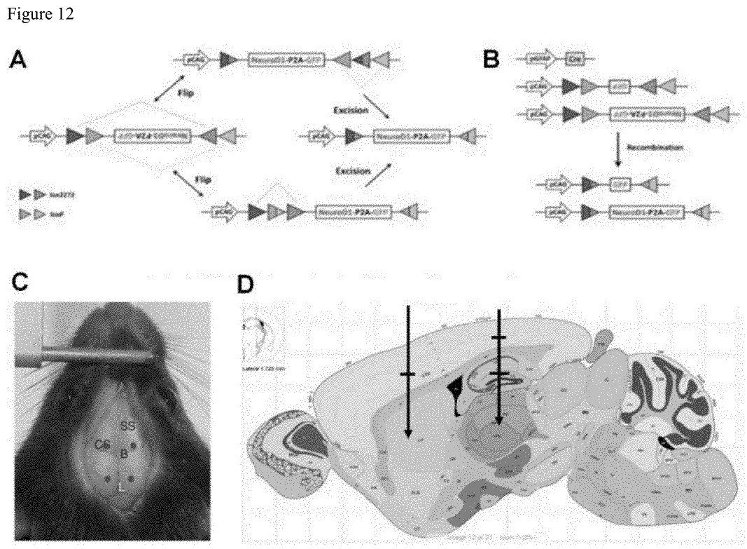

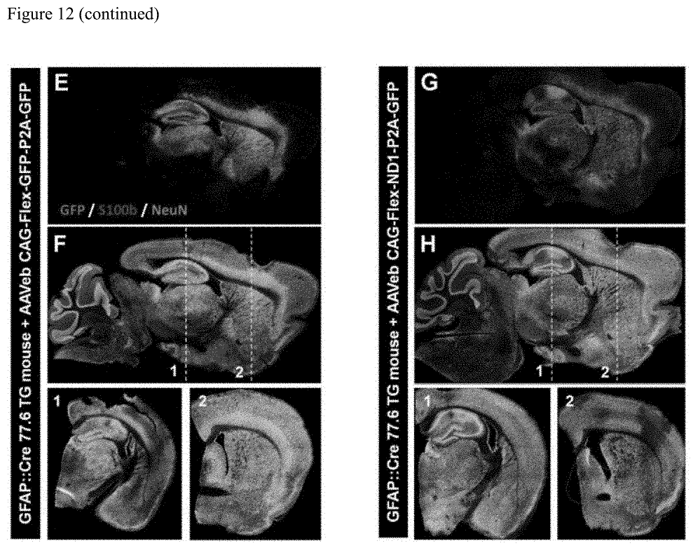

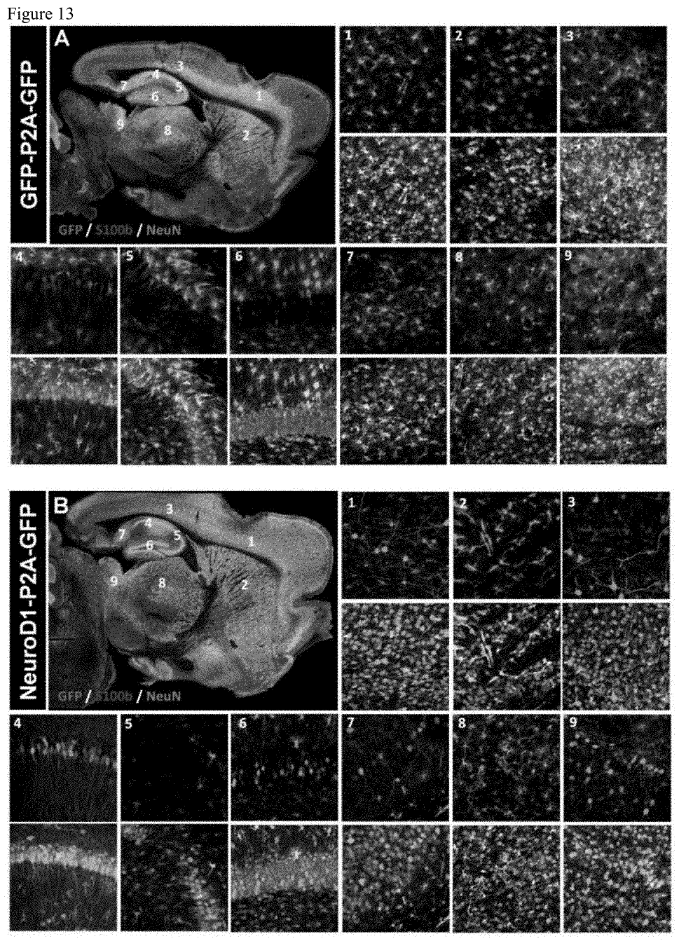

To globally target astrocytes for neuronal conversion in the mouse brain, the AAV-GFAP::Cre FLEX system (FIG. 12A and FIG. 12B) and multiple intracranial injections (FIG. 12 C and FIG. 12 D) were applied in our study. AAV-PHP.eB was selected to ectopically express NeuroD1 and GFP (control) in the mouse brain. We injected the FLEX GFP and NeuroD1 AAV-PHP.eB into the GFAP::Cre transgenic mouse brain. 15 days post injection (dpi), the mouse brain was sliced for histologic analysis. Immunohistochemical analyses of sagittal and coronal sections around the injected regions showed that GFP positive cells were detectable in a broad area both in GFP and NeuroD1-GFP injected mouse brain. These results indicate that multiple intracranial injections of AAV-PHP.eB achieve the broad infection through the mouse brain.

### Global Astrocytes-to-Neurons Conversion in GFAP::Cre Transgenic Mouse Brain

To test whether NeuroD1 can convert astrocytes into neurons globally, we inspected the different areas of the mouse brain by immunostaining. We found that almost all of the GFP positive cells were co-labeled with the astrocytic marker S1000 in the GFP treated mouse (FIG. 13A). Interestingly, we observed a number of GFP positive cells that were co-labeled with the neuronal marker NeuN in NeuroD1 treated mouse brain, including cortical area, hippocampus, subiculum, and middle brain (FIG. 13B).

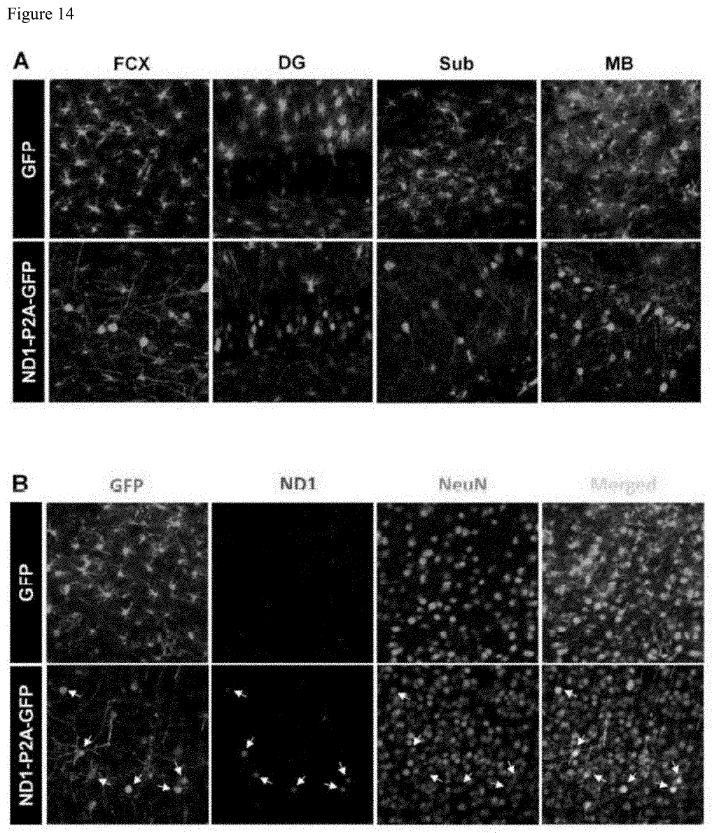

To directly show the different morphology of GFP positive cells between control and NeuroD1 treated mouse brain, the high magnification images were exhibited one by one. The majority of the GFP positive cells in NeuroD1 group exhibited the typical neuronal morphology, however, the GFP positive cells in control group exhibited the typical astrocytic morphology (FIG. 14A). Furthermore, we also detected that NeuroD1 were over-expressed in the converted neurons (FIG. 14B). These results indicate that GFAP::Cre FLEX system specifically target astrocytes and multiple intracranial injections of AAV-PHP.eB NeuroD1 achieve the broad astrocytes-to-neurons conversion across the mouse brain.

### Global Astrocytes-to-Neurons Conversion in 5×FAD Transgenic Mouse Brain

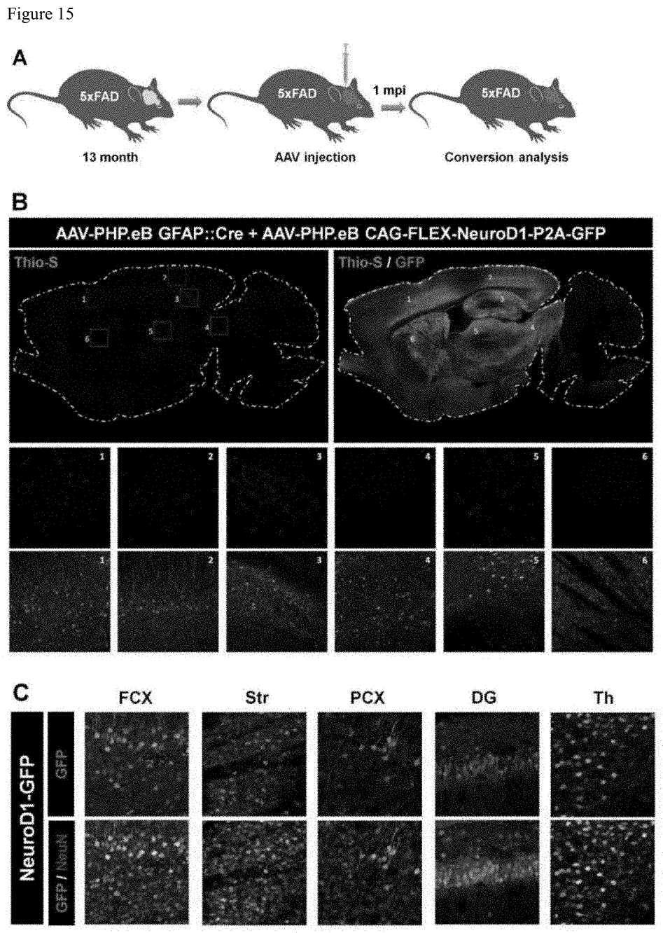

To achieve the global conversion in the AD mouse brain, we applied the global conversion system into the 5×FAD mouse brain. AAV-PHP.eB GFAP::Cre virus were co-injected with FLEX NeuroD1-P2A-GFP into a 13-month-old 5×FAD mouse brain, 1 month later the mouse was sacrificed for conversion analysis (FIG. 15A). The broad Aβ plaques were detected by the thioflavin-S staining (which, when viewed in color, stained blue, FIG. 15B). Interestingly, almost the whole cerebral area was covered by the GFP (FIG. 15B), which indicates that the infection efficiency of AAV-PHP.eB was pretty high in 5×FAD mouse brain. To examine the astrocytes-to-neurons conversion in 5×FAD mouse brain, GFP and NeuN immunostaining were employed. Many GFP positive cells were co-localized with NeuN through the different brain areas (FIG. 15C). These data demonstrate that the global astrocytes-to-neurons conversion also works in the 5×FAD mouse model brain. In addition, the results presented herein demonstrate that new neurons and new astrocytes were reaching homeostasis.

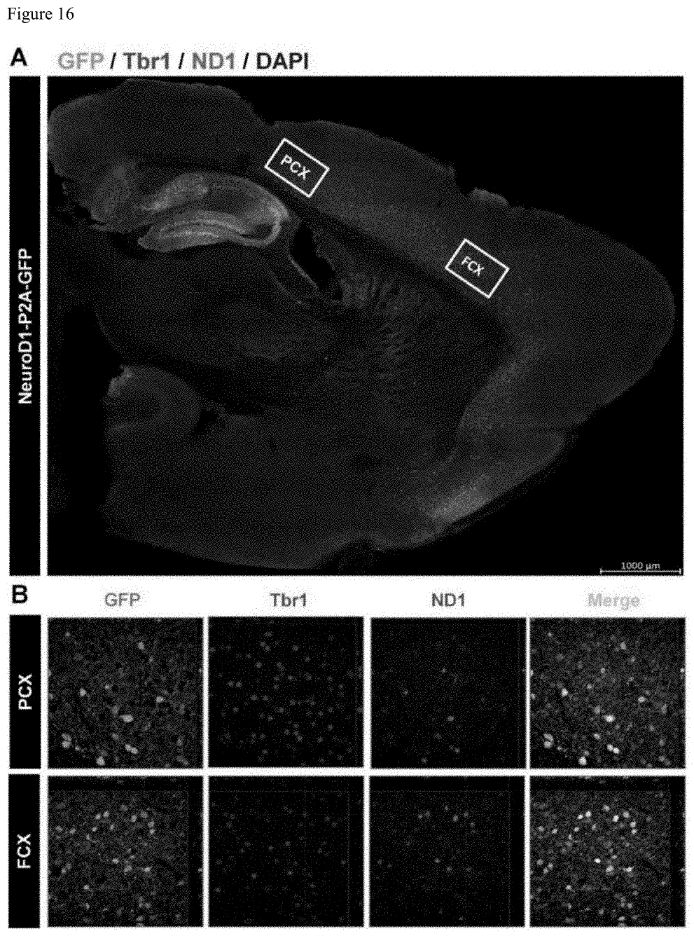

The brain is a highly complex but organized organ, and the different subtypes of the neurons have their particular location in the brain. For example, layer and neuronal subtype specificity have been identified within the cerebral cortex. To examine whether global conversion can generate the right neurons in the right place in the cerebral cortex, we investigated a typical cortical marker Tbr1 express pattern after global conversion. Tbr1 is highly expressed in the cortical layer II/III and VI neurons. Interestingly, we also found that the Tbr1 was expressed in NeuroD1 converted neurons that were located in the deep layer of the cerebral cortex (FIG. 16A and FIG. 16B). These results suggest that the global conversion can generate the specific subtype of cortical neurons in the right position.

### Example 3—Global Conversion of Astrocytes into Neurons Through Retro-Orbital Injection of NeuroD1 AAV

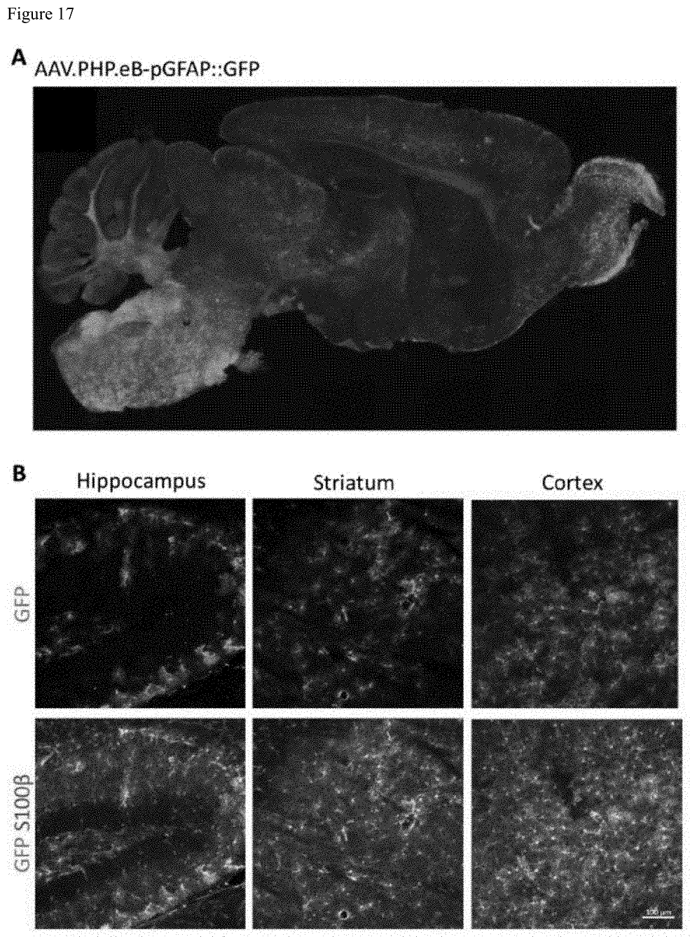

By using AAV.PHP.eB, a serotype of AAV that is recently discovered (Chan et al., Nat. Neurosci., 20(8):1172-1179 (2017)), we are able to efficiently transduce the mouse brain across the blood-brain-barrier (BBB) by intravenous injection. We firstly packaged AAV.PHP.eB with GFAP promoter-driven GFP plasmid. At 17 days after retro-orbital injection, the mouse brain was widely labeled by GFP fluorescence (FIG. 17A). Co-immunostaining with astrocytic marker, S100β, showed very specific expression of GFP in cortex, striatum, and hippocampus regions.

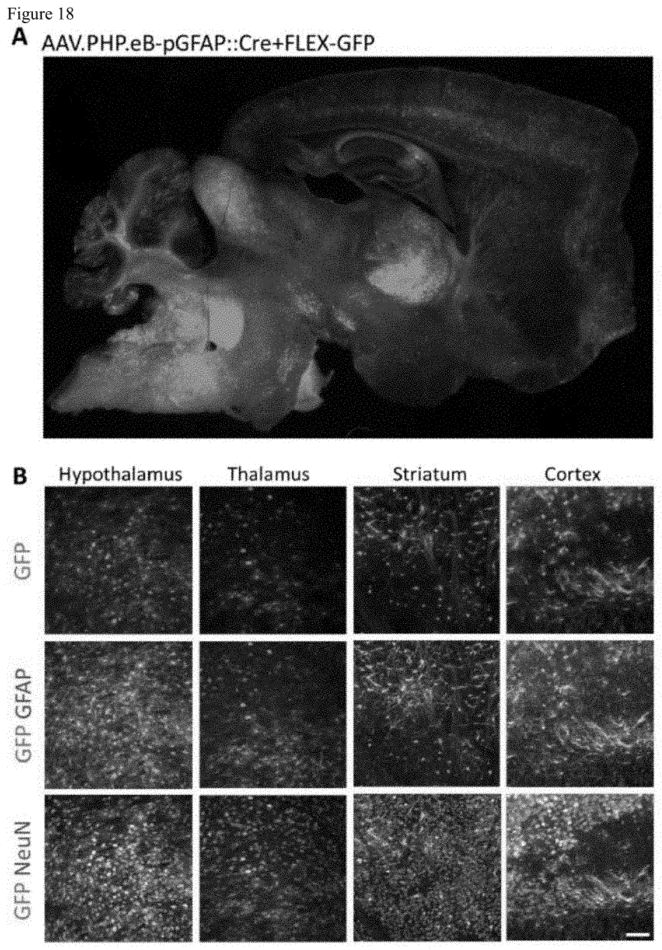

We next packaged the AAV.PHP.eB virus with Cre-FLEX system, trying to achieve higher expression of the interested genes. We firstly made AAV.PHP.eB with GFAP::Cre and FLEX-GFP virus. Retro-orbital injection of this combination also showed wide infection and strong expression in the brain (FIG. 18A). While many GFP positive cells in different brain regions showed astrocyte morphology and GFAP signal, some of them also showed neuronal morphology and colocalization with NeuN (FIG. 18B).

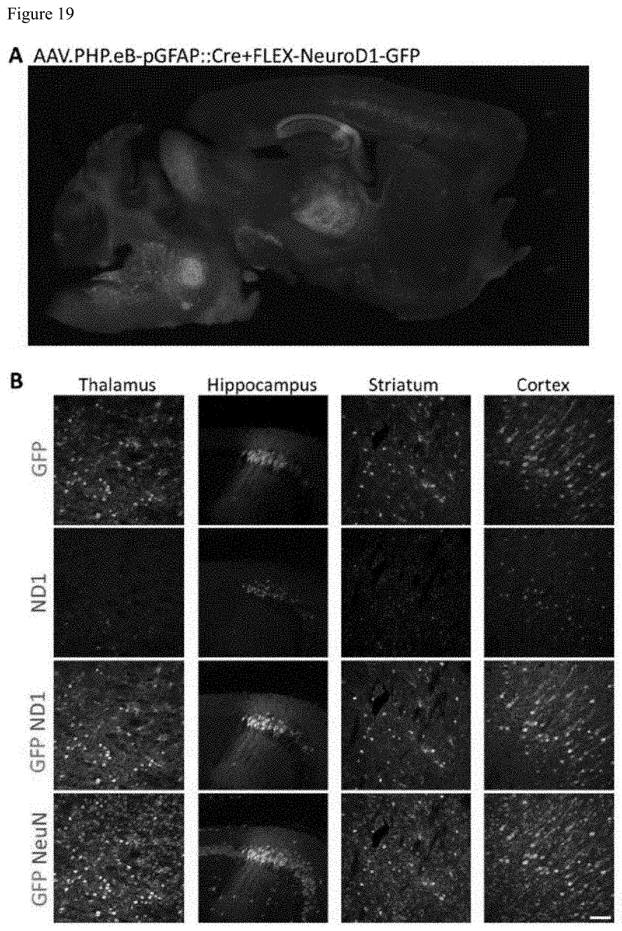

We also put the Cre-FLEX-NeuroD1 system into the AAV.PHP.eB virus. After retro-orbital injection of AAV.PHP.eB with GFAP::Cre and FLEX-NeuroD1-GFP virus, both GFP and NeuroD1 signals were detected in different regions of the brain (FIG. 19). Interestingly, many of the GFP and NeuroD1 signals were colocalized with neuronal marker NeuN, suggesting we could achieve systematic infection and astrocyte-to-neuron conversion through intravenous injection of AAV.PHP.eB virus. These result demonstrate that a non-invasive therapy of delivering NeuroD1 to astrocytes can be used to globally regenerate neurons in many neurodegenerative diseases such as AD and large scale stroke.

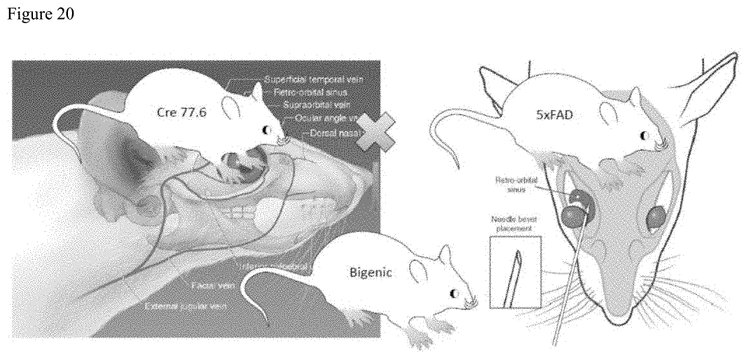

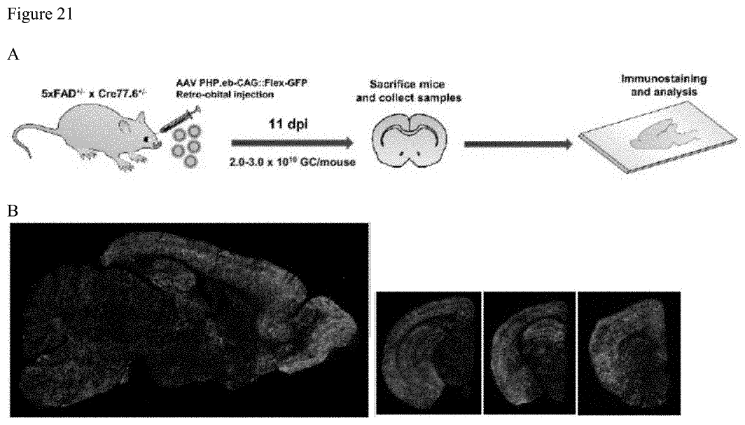

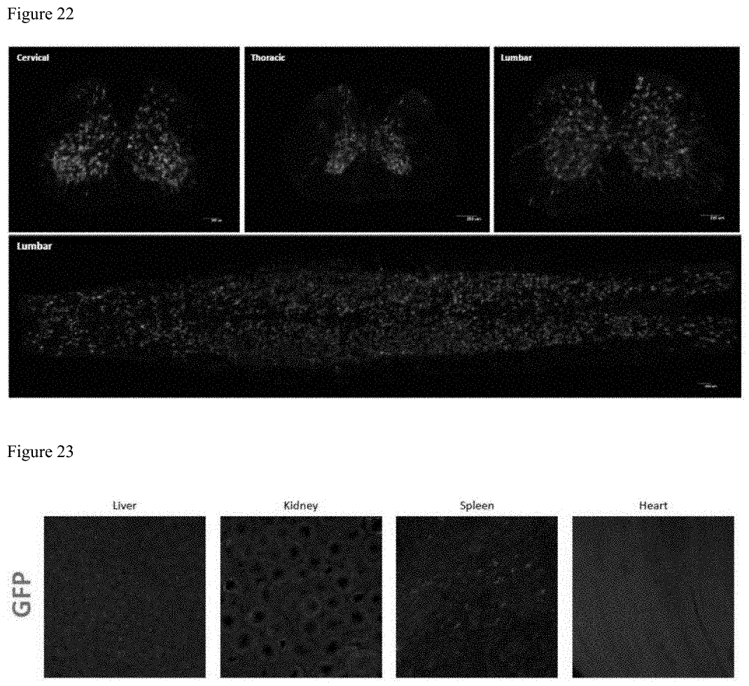

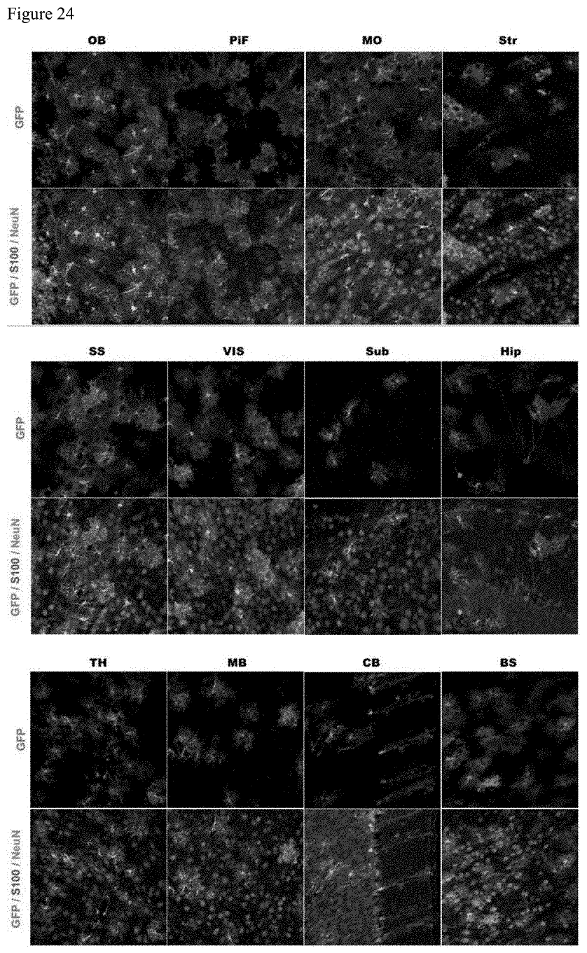

FIG. 20 demonstrates the retro-orbital (r.o.) injection of a virus (e.g., AAV.PHP.eb-CAG::Flex-GFP) for global targeting of astrocytes as confirmed by GFP staining within brain (FIG. 21A-21B), different regions of the brain (FIG. 24), and spinal cord (FIG. 22). No obvious GFP signals were detected in other organs (FIG. 23).

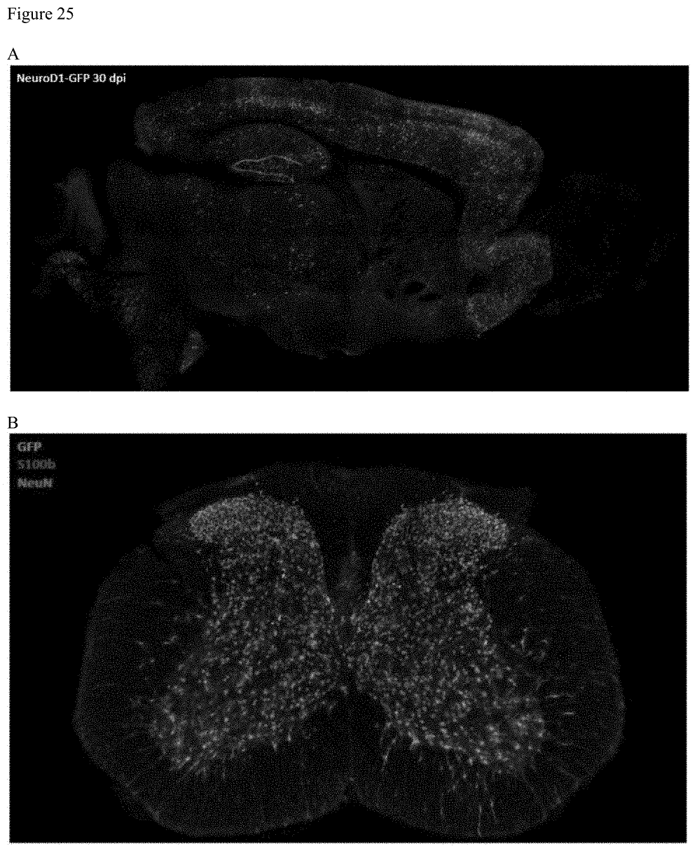

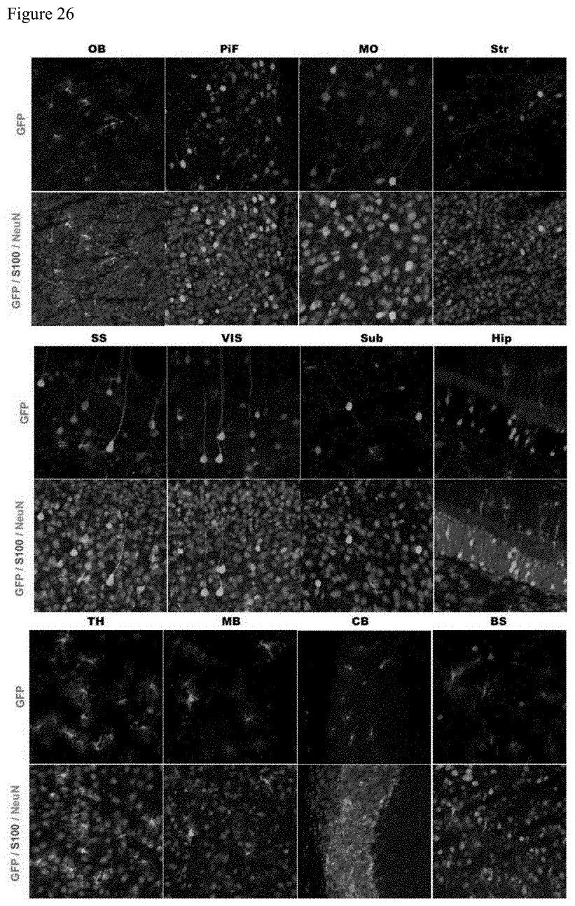

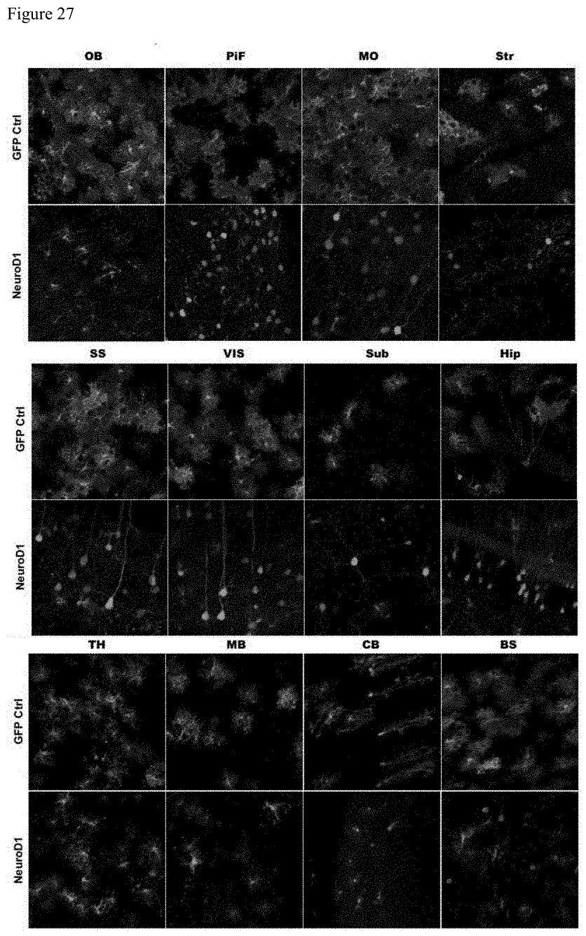

When a virus designed to express NeuroD1 (AAV.PHP.eb-CAG::Flex-ND1-P2A-GFP; about 2.0×10¹⁰ genome copies/mouse) was injected via retro-orbital injection, astrocytes were converted into neurons within the cerebrum, but in the spinal cord it seems to take longer time for conversion to occur (FIG. 25A and FIG. 25B). This global conversion of astrocytes into neurons also was observed in different regions of the brain with different efficiency (FIGS. 26 and 27).

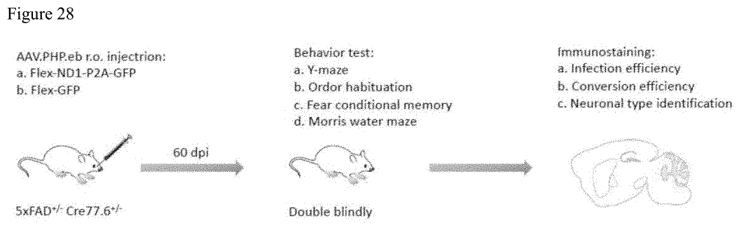

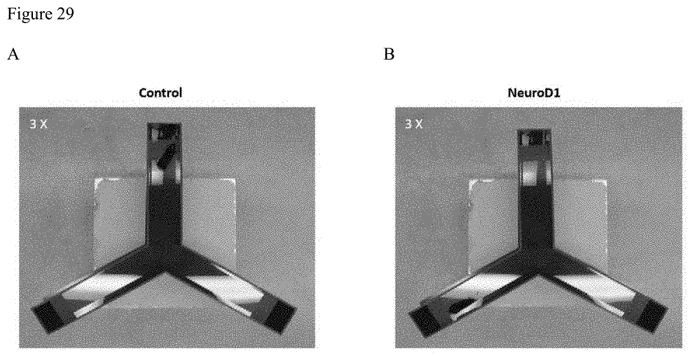

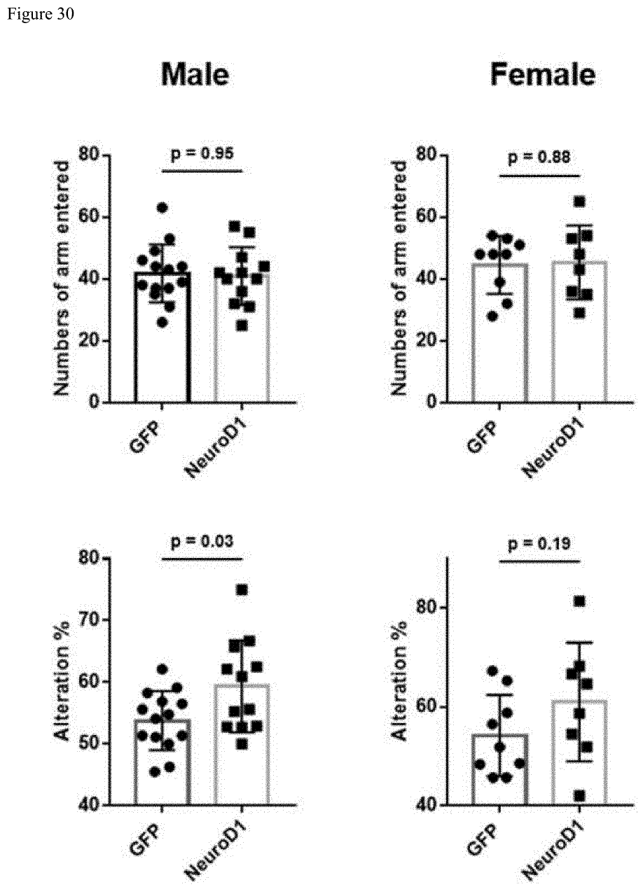

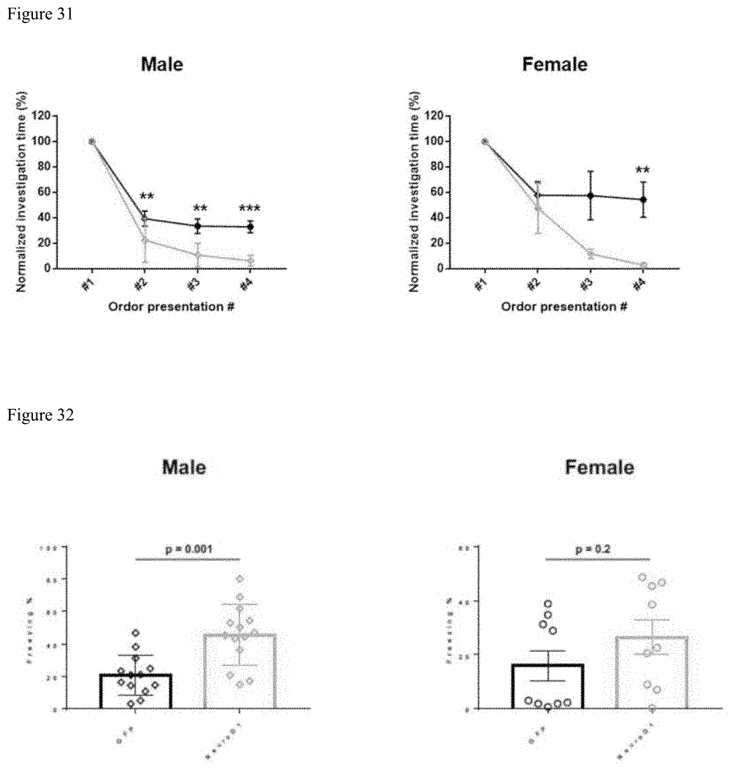

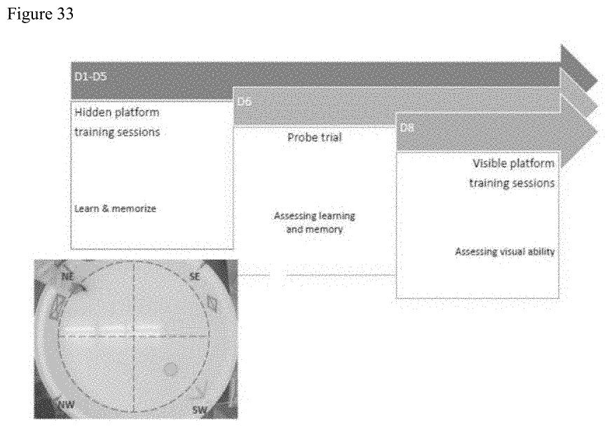

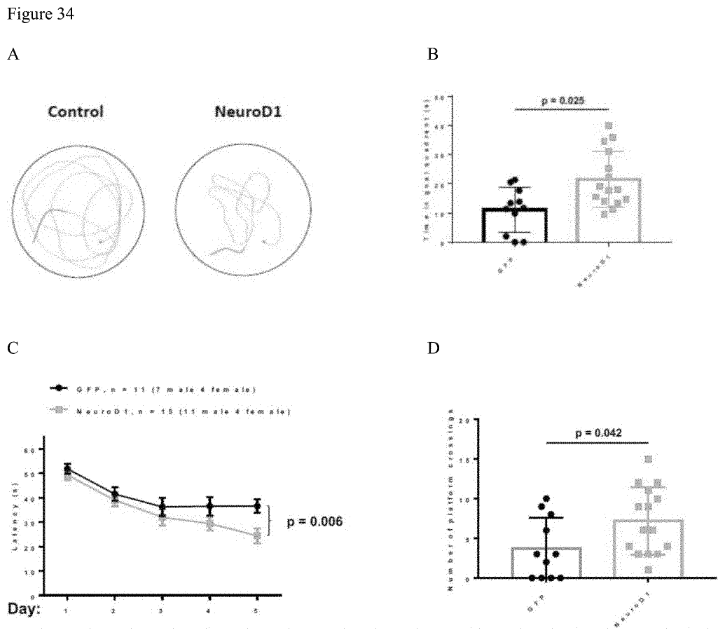

Retro-orbital injection of virus designed to express NeuroD1 (AAV.PHP.eb-CAG::Flex-ND1-P2A-GFP; about 2.0×10¹⁰ genome copies/mouse) successfully improved memory in an animal model of AD (FIG. 28) In particular, retro-orbital injection of virus designed to express NeuroD1 resulted in memory improvement as assessed using a Y maze memory assessment assay (FIG. 29A-FIG. 29B and FIG. 30), as assessed using an odor habituation assay (FIG. 31), as assessed using a fear conditional memory test (FIG. 32), and as assessed using a Morris Water Maze for assessing spatial learning and memory (FIG. 33 and FIG. 34A-FIG. 34D).

These results demonstrate that AAV PHP.eb retro-orbital injection is a good method to globally target astrocytes for conversion into neurons. These results also demonstrate that the efficiency of NeuroD1-mediated astrocyte-to-neuron conversion is different in different brain regions and that cortical and hippocampal astrocytes can be converted into neurons by NeuroD1 with high efficiency. In addition, these results demonstrate that global conversion of astrocytes into neurons can improve learning and memory performance in a mouse model for AD (i.e., 5×FAD mice).

It is very hard to produce billions of new neurons in the whole Alzheimer's brain by neural stem cell transplantation, which encouraged us to invent an alternative way to globally regenerate billions of neurons inside the Alzheimer's brain. As described herein, we developed a gene therapy strategy that can globally target astrocytes for in situ neuronal conversion in 5×FAD mice brain by retro-orbital injection of AAV PHP.eb virus. When NeuroD1 was overexpressed in those astrocytes, we found large numbers of converted neurons in different brain regions, especially in cortex and hippocampus. So far, through the global astrocyte-to-neuron conversion in 5×FAD mouse brain, we conclude some interesting findings. After retro-orbital injection of AAV PHP.eb virus into the 5×FAD^{+/-}/Cre77.6^{+/-} bigenic mice, we found that the reporter gene GFP was widely expressed in astrocytes throughout the different brain and spinal cord regions. 30 days post AAV PHP.eb-CAG::Flex-NeuroD1-P2A-GFP injection, we observed many GFP positive converted neurons in 5×FAD mouse brain. The efficiency of the NeuroD1-mediated astrocyte-to-neuron conversion is about 70-90 percent in cerebral cortex, piriform cortex, and hippocampus in 5×FAD mouse brain. 5×FAD^{+/-}/Cre77.6^{+/-} bigenic mice were treated with GFP or NeuroD1-GFP at age of 6 months. Then, we performed a series of behavior tests at age of 8 months (2 months post AAV PHP.eb injection) and found that global astrocyte-to-neuron conversion significantly improved learning and memory performance in 5×FAD mice as assessed by a Y-Maze assay, fear conditioning memory, odor habituation, and a Morris water maze.

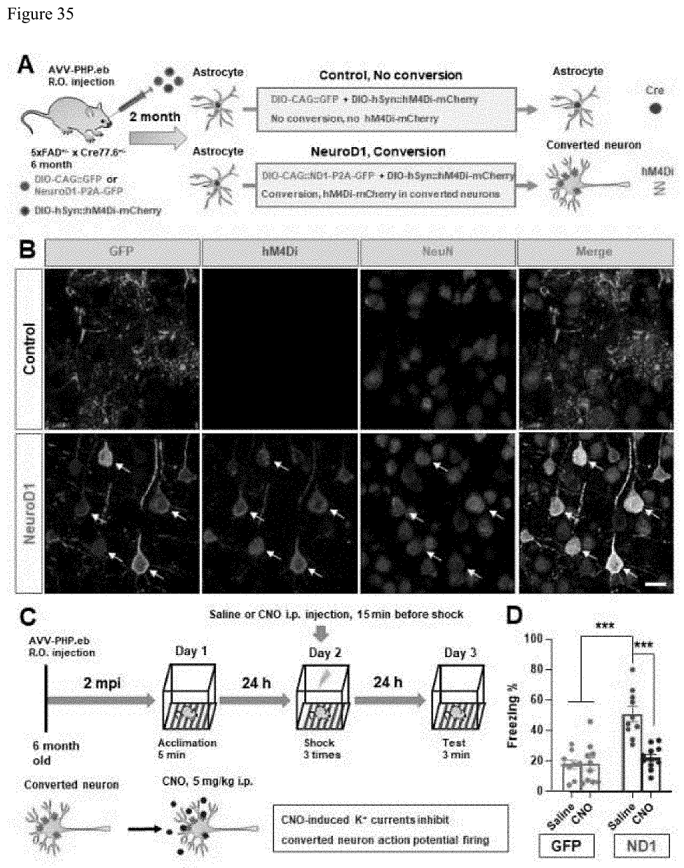

After NeuroD1 treatment exhibited improvement of cognitive functions in AD mice, we further designed experiments to inhibit the NeuroD1-converted neurons and examined whether the enhancement of memory would be reduced accordingly. For this purpose, we employed a chemogenetic method by expressing an inhibitory receptor hM4Di together with NeuroD1 so that converted neurons will express hM4Di. After confirming that NeuroD1-treatment enhanced the fear conditioning memory in AD mice, CNO was applied to activate the hM4Di receptors so that NeuroD1-converted neurons were inhibited from firing action potentials. Interestingly, we found that the memory enhancement was abolished after CNO silenced the NeuroD1-converted neurons (FIG. 35A-FIG. 35D). These results further demonstrate that the NeuroD1-converted neurons contributed to the memory enhancement in the AD mice.

### Example 4—Additional Embodiments

Embodiment 1. A method for treating a mammal having a neurological disorder in the brain, wherein said method comprises administering a composition comprising exogenous nucleic acid encoding a Neurogenic Differentiation 1 (NeuroD1) polypeptide or a biologically active fragment thereof to the brain of said mammal.
Embodiment 2. The method of embodiment 1, wherein said mammal is a human.
Embodiment 3. The method of embodiment 1, wherein said neurological disorder is Alzheimer's disease.
Embodiment 4. The method of embodiment 1, wherein said administering step comprises delivering an expression vector comprising a nucleic acid encoding NeuroD1 to the brain.
Embodiment 5. The method of embodiment 1, wherein said administering step comprises delivering a recombinant viral expression vector comprising a nucleic acid encoding NeuroD1 to the brain.
Embodiment 6. The method of embodiment 1, wherein said administering step comprises delivering a recombinant adeno-associated virus expression vector comprising a nucleic acid encoding NeuroD1 to the brain.
Embodiment 7. The method of embodiment 6, wherein the adeno-associated virus is an AAV.PHP.eB.
Embodiment 8. The method of any of embodiments 1-7, wherein said administering step comprises administering a recombinant expression vector comprising a nucleic acid sequence encoding NeuroD1 protein, wherein the nucleic acid sequence encoding NeuroD1 protein comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO:2 or SEQ ID NO:4, or a functional fragment thereof.
Embodiment 9. The method of any of embodiments 1-8, wherein said administering step comprises a stereotactic intracranial injection.
Embodiment 10. The method of embodiment 9, wherein said administering step comprises two or more stereotactic intracranial injections.
Embodiment 11. The method of any one of embodiments 1-8, wherein said administering step comprises an extracranial injection.
Embodiment 12. The method of embodiment 11, wherein said administering step comprises two or more extracranial injections.
Embodiment 13. The method of any one of embodiments 1-8, wherein said administering step comprises a retro-orbital injection.
Embodiment 14. A method of treating a mammal having Alzheimer's disease, wherein said method comprises administering a pharmaceutical composition comprising a pharmaceutically acceptable carrier containing adeno-associated virus particles comprising a nucleic acid encoding a NeuroD1 polypeptide or a biologically active fragment thereof to the brain of said mammal.
Embodiment 15. The method of embodiment 14, wherein the pharmaceutical composition comprises about 1 μL to about 500 μL of a pharmaceutically acceptable carrier containing adeno-associated virus particles at a concentration of 10¹⁰-10¹⁴ adeno-associated virus particles/mL of carrier.
Embodiment 16. The method of embodiment 14 or 15, wherein the pharmaceutical composition is injected in the brain of said mammal at a controlled flow rate of about 0.1 μL/minute to about 5 μL/minute.
Embodiment 17. A method for (1) reducing neurofibrillary tangles of hyperphosphorylated tau protein, (2) reducing aggregation of extracellular amyloid plaques, (3) reducing neuroinflammation, (4) reducing interleukin 1β (IL-1β), (5) generating new glutamatergic neurons, (6) increasing survival of GABAergic neurons, (7) generating new non-reactive astrocytes, (8) reducing the number of reactive astrocytes, or (9) improving memory within a mammal having Alzheimer's disease and in need of said (1), (2), (3), (4), (5), (6), (7), (8) or (9), wherein said method comprises administering a composition comprising exogenous nucleic acid encoding a NeuroD1 polypeptide or a biologically active fragment thereof to said mammal, wherein said (1) hyperphosphorylated neurofibrillary tau protein tangles are reduced, (2) aggregation of extracellular amyloid plaques is reduced, (3) neuroinflammation is reduced, (4) interleukin 1β (IL-1β) levels are reduced, (5) new glutamatergic neurons are generated, (6) survival of GABAergic neurons is increased, (7) new non-reactive astrocytes are generated, (8) the number of reactive astrocytes is reduced, or (9) said memory is improved.
Embodiment 18. The embodiment of 17, wherein said mammal is a human.
Embodiment 19. The method of any one of embodiments 17-18, wherein said administering step comprises delivering an expression vector comprising a nucleic acid encoding a NeuroD1 polypeptide.
Embodiment 20. The method of any one of embodiments 17-19, wherein said administering step comprises delivering a recombinant viral expression vector comprising a nucleic acid encoding a NeuroD1 polypeptide.
Embodiment 21. The method of any one of embodiments 17-20, wherein said administering step comprises delivering a recombinant adeno-associated virus expression vector comprising a nucleic acid encoding a NeuroD1 polypeptide.
Embodiment 22. The method of embodiment 21, wherein said recombinant adeno-associated virus expression vector is an AAV.PHP.eB expression vector.
Embodiment 23. The method of any of embodiments 17-22, wherein said administering step comprises administering a recombinant expression vector comprising a nucleic acid sequence encoding a NeuroD1 polypeptide, wherein said nucleic acid sequence encoding a NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO:2 or SEQ ID NO:4, or a functional fragment thereof.
Embodiment 24. The method of any of embodiments 17-23, wherein said administering step comprises a stereotactic intracranial injection.
Embodiment 25. The method of embodiment 24, wherein said administering step comprises two or more stereotactic intracranial injections.
Embodiment 26. The method of any one of embodiments 17-23, wherein said administering step comprises an extracranial injection.
Embodiment 27. The method of embodiment 26, wherein said administering step comprises two or more extracranial injections.
Embodiment 28. The method of any one of embodiments 17-23, wherein said administering step comprises a retro-orbital injection.

SEQUENCES

SEQ ID NO: 1-Human NeuroD1 nucleic acid sequence encoding human
NeuroD1 protein-1071 nucleotides, including stop codon
ATGACCAAATCGTACAGCGAGAGTGGGCTGATGGGCGAGCCTCAGCCCCAAGGTCCTCCAAGCT
GGACAGACGAGTGTCTCAGTTCTCAGGACGAGGAGCACGAGGCAGACAAGAAGGAGGACGACCT
CGAAGCCATGAACGCAGAGGAGGACTCACTGAGGAACGGGGGAGAGGAGGAGGACGAAGATGAG
GACCTGGAAGAGGAGGAAGAAGAGGAAGAGGAGGATGACGATCAAAAGCCCAAGAGACGCGGCC
CCAAAAAGAAGAAGATGACTAAGGCTCGCCTGGAGCGTTTTAAATTGAGACGCATGAAGGCTAA
CGCCCGGGAGCGGAACCGCATGCACGGACTGAACGCGGCGCTAGACAACCTGCGCAAGGTGGTG
CCTTGCTATTCTAAGACGCAGAAGCTGTCCAAAATCGAGACTCTGCGCTTGGCCAAGAACTACA
TCTGGGCTCTGTCGGAGATCCTGCGCTCAGGCAAAAGCCCAGACCTGGTCTCCTTCGTTCAGAC
GCTTTGCAAGGGCTTATCCCAACCCACCACCAACCTGGTTGCGGGCTGCCTGCAACTCAATCCT
CGGACTTTTCTGCCTGAGCAGAACCAGGACATGCCCCCCCACCTGCCGACGGCCAGCGCTTCCT
TCCCTGTACACCCCTACTCCTACCAGTCGCCTGGGCTGCCCAGTCCGCCTTACGGTACCATGGA
CAGCTCCCATGTCTTCCACGTTAAGCCTCCGCCGCACGCCTACAGCGCAGCGCTGGAGCCCTTC
TTTGAAAGCCCTCTGACTGATTGCACCAGCCCTTCCTTTGATGGACCCCTCAGCCCGCCGCTCA
GCATCAATGGCAACTTCTCTTTCAAACACGAACCGTCCGCCGAGTTTGAGAAAAATTATGCCTT
TACCATGCACTATCCTGCAGCGACACTGGCAGGGGCCCAAAGCCACGGATCAATCTTCTCAGGC
ACCGCTGCCCCTCGCTGCGAGATCCCCATAGACAATATTATGTCCTTCGATAGCCATTCACATC
ATGAGCGAGTCATGAGTGCCCAGCTCAATGCCATATTTCATGATTAG
SEQ ID NO: 2-Human NeuroD1 amino acid sequence-356 amino acids
encoded by SEQ ID NO: 1
MTKSYSESGLMGEPQPQGPPSWTDECLSSQDEEHEADKKEDDLEAMNAEEDSLRNGGEEEDEDE
DLEEEEEEEEEDDDQKPKRRGPKKKKMTKARLERFKLRRMKANARERNRMHGLNAALDNLRKVV
PCYSKTQKLSKIETLRLAKNYIWALSEILRSGKSPDLVSFVQTLCKGLSQPTTNLVAGCLQLNP
RTFLPEQNQDMPPHLPTASASFPVHPYSYQSPGLPSPPYGTMDSSHVFHVKPPPHAYSAALEPF
FESPLTDCTSPSFDGPLSPPLSINGNFSFKHEPSAEFEKNYAFTMHYPAATLAGAQSHGSIFSG
TAAPRCEIP1DNIMSFDSHSHHERVMSAQLNAIFHD
SEQ ID NO: 3-Mouse NeuroD1 nucleic acid sequence encoding mouse
NeuroD1 protein-1074 nucleotides, including stop codon
ATGACCAAATCATACAGCGAGAGCGGGCTGATGGGCGAGCCTCAGCCCCAAGGTCCCCCAAGCT
GGACAGATGAGTGTCTCAGTTCTCAGGACGAGGAACACGAGGCAGACAAGAAAGAGGACGAGCT
TGAAGCCATGATGCAGAGGAGGACTCTCTGAGAAACGGGGGAGAGGAGGAGGAGGAAGATGAG
GATCTAGAGGAAGAGGAGGAAGAAGAAGAGGAGGAGGAGGATCAAAAGCCCAAGAGACGGGGTC
CCAAAAAGAAAAAGATGACCAAGGCGCGCCTAGAACGTTTTAAATTAAGGCGCATGAAGGCCAA
CGCCCGCGAGCGGAACCGCATGCACGGGCTGAACGCGGCGCTGGACAACCTGCGCAAGGTGGTA
CCTTGCTACTCCAAGACCCAGAAACTGTCTAAAATAGAGACACTGCGCTTGGCCAAGAACTACA
TCTGGGCTCTGTCAGAGATCCTGCGCTCAGGCAAAAGCCCTGATCTGGTCTCCTTCGTACAGAC
GCTCTGCAAAGGTTTGTCCCAGCCCACTACCAATTTGGTCGCCGGCTGCCTGCAGCTCAACCCT
CGGACTTTCTTGCCTGAGCAGAACCCGGACATGCCCCCGCATCTGCCAACCGCCAGCGCTTCCT
TCCCGGTGCATCCCTACTCCTACCAGTCCCCTGGACTGCCCAGCCCGCCCTACGGCACCATGGA
CAGCTCCCACGTCTTCCACGTCAAGCCGCCGCCACACGCCTACAGCGCAGCTCTGGAGCCCTTC
TTTGAAAGCCCCCTAACTGACTGCACCAGCCCTTCCTTTGACGGACCCCTCAGCCCGCCGCTCA
GCATCAATGGCAACTTCTCTTTCAAACACGAACCATCCGCCGAGTTTGAAAAAAATTATGCCTT
TACCATGCACTACCCTGCAGCGACGCTGGCAGGGCCCCAAAGCCACGGATCAATCTTCTCTTCC
GGTGCCGCTGCCCCTCGCTGCGAGATCCCCATAGACAACATTATGTCTTTCGATAGCCATTCGC
ATCATGAGCGAGTCATGAGTGCCCAGCTTAATGCCATCTTTCACGATTAG
SEQ ID NO: 4-Mouse NeuroD1 amino acid sequence-357 amino acids
encoded by SEQ ID NO: 3
MTKSYSESGLMGEPQPQGPPSWTDECLSSQDEEHEADKKEDELEAMNAEEDSLRNGGEEEEEDE
DLEEEEEEEEEEEDQKPKRRGPKKKKMTKARLERFKLRRMKANARERNRMHGLNAALDNLRKVV
PCYSKTQKLSKIETLRLAKNYIWALSEILRSGKSPDLVSFVQTLCKGLSQPTTNLVAGCLQLNP
RTFLPEQNPDMPPHLPTASASFPVHPYSYQSPGLPSPPYGTMDSSHVFHVKPPPHAYSAALEPF
FESPLTDCTSPSFDGPLSPPLSINGNFSFKHEPSAEFEKNYAFTMHYPAATLAGPQSHGSIFSS
GAAAPRCEIP1DNIMSFDSHSHHERVMSAQLNAIFHD
Mouse LCN2 promoter-SEQ ID NO: 5
GCAGTGTGGAGACACACCCACTTTCCCCAAGGGCTCCTGCTCCCCCAAGTGATCCCCTTATCCT
CCGTGCTAAGATGACACCGAGGTTGCAGTCCTTACCTTTGAAAGCAGCCACAAGGGCGTGGGGG
TGCACACCTTTAATCCCAGCACTCGGGAGGCAGAGGCAGGCAGATTTCTGAGTTCGAGACCAGC
CTGGTCTACAAAGTGAATTCCAGGACAGCCAGGGCTATACAGAGAAACCCTGTCTTGAAAAAAA
AAGAGAAAGAAAAAAGAAAAAAAAAAATGAAAGCAGCCACATCTAAGGACTACGTGGCACAGGA
GAGGGTGAGTCCCTGAGAGTTCAGCTGCTGCCCTGTCTGTTCCTGTAAATGGCAGTGGGGTCAT
GGGAAAGTGAAGGGGCTCAAGGTATTGGACACTTCCAGGATAATCTTTTGGACGCCTCACCCTG
TGCCAGGACCAAGGCTGAGCTTGGCAGGCTCAGAACAGGGTGTCCTGTTCTTCCCTGTCTAAAA
CATTCACTCTCAGCTTGCTCACCCTTCCCCAGACAAGGAAGCTGCACAGGGTCTGGTGTTCAGA
TGGCTTTGGCTTACAGCAGGTGTGGGTGTGGGGTAGGAGGCAGGGGGTAGGGGTGGGGGAAGCC
TGTACTATACTCACTATCCTGTTTCTGACCCTCTAGGACTCCTACAGGGTTATGGGAGTGGACA
GGCAGTCCAGATCTGAGCTGCTGACCCACAAGCAGTGCCCTGTGCCTGCCAGAATCCAAAGCCC
TGGGAATGTCCCTCTGGTCCCCCTCTGTCCCCTGCAGCCCTTCCTGTTGCTCAACCTTGCACAG
TTCCGACCTGGGGGAGAGAGGGACAGAAATCTTGCCAAGTATTTCAACAGAATGTACTGGCAAT
TACTTCATGGCTTCCTGGACTTGGTAAAGGATGGACTACCCCGCCCAACAGGGGGGCTGGCAGC
CAGGTAGGCCCATAAAAAGCCCGCTGGGGAGTCCTCCTCACTCTCTGCTCTTCCTCCTCCAGCA
CACATCAGACCTAGTAGCTGTGGAAACCA
Human GFAP promoter-SEQ ID NO: 6
GTCTGCAAGCAGACCTGGCAGCATTGGGCTGGCCGCCCCCCAGGGCCTCCTCTTCATGCCCAGT
GAATGACTCACCTTGGCACAGACACAATGTTCGGGGTGGGCACAGTGCCTGCTTCCCGCCGCAC
CCCAGCCCCCCTCAAATGCCTTCCGAGAAGCCCATTGAGTAGGGGGCTTGCATTGCACCCCAGC
CTGACAGCCTGGCATCTTGGGATAAAAGCAGCACAGCCCCCTAGGGGCTGCCCTTGCTGTGTGG
CGCCACCGGCGGTGGAGAACAAGGCTCTATTCAGCCTGTGCCCAGGAAAGGGGATCAGGGGATG
CCCAGGCATGGACAGTGGGTGGCAGGGGGGGAGAGGAGGGCTGTCTGCTTCCCAGAAGTCCAAG
GACACAAATGGGTGAGGGGACTGGGCAGGGTTCTGACCCTGTGGGACCAGAGTGGAGGGCGTAG
ATGGACCTGAAGTCTCCAGGGACAACAGGGCCCAGGTCTCAGGCTCCTAGTTGGGCCCAGTGGC
TCCAGCGTTTCCAAACCCATCCATCCCCAGAGGTTCTTCCCATCTCTCCAGGCTGATGTGTGGG
AACTCGAGGAAATAAATCTCCAGTGGGAGACGGAGGGGTGGCCAGGGAAACGGGGCGCTGCAGG
AATAAAGACGAGCCAGCACAGCCAGCTCATGCGTAACGGCTTTGTGGAGCTGTCAAGGCCTGGT
CTCTGGGAGAGAGGCACAGGGAGGCCAGACAAGGAAGGGGTGACCTGGAGGGACAGATCCAGGG
GCTAAAGTCCTGATAAGGCAAGAGAGTGCCGGCCCCCTCTTGCCCTATCAGGACCTCCACTGCC
ACATAGAGGCCATGATTGACCCTTAGACAAAGGGCTGGTGTCCAATCCCAGCCCCCAGCCCCAG
AACTCCAGGGAATGAATGGGCAGAGAGCAGGAATGTGGGACATCTGTGTTCAAGGGAAGGACTC
CAGGAGTCTGCTGGGAATGAGGCCTAGTAGGAAATGAGGTGGCCCTTGAGGGTACAGAACAGGT
TCATTCTTCGCCAAATTCCCAGCACCTTGCAGGCACTTACAGCTGAGTGAGATAATGCCTGGGT
TATGAAATCAAAAAGTTGGAAAGCAGGTCAGAGGTCATCTGGTACAGCCCTTCCTTCCCTTTTT
TTTTTTTTTTTTTTGTGAGACAAGGTCTCTCTCTGTTGCCCAGGCTGGAGTGGCGCAAACACAG
CTCACTGCAGCCTCAACCTACTGGGCTCAAGCAATCCTCCAGCCTCAGCCTCCCAAAGTGCTGG
GATTACAAGCATGAGCCACCCCACTCAGCCCTTTCCTTCCTTTTTAATTGATGCATAATAATTG
TAAGTATTCATCATGGTCCAACCAACCCTTTCTTGACCCACCTTCCTAGAGAGAGGGTCCTCTT
GATTCAGCGGTCAGGGCCCCAGACCCATGGTCTGGCTCCAGGTACCACCTGCCTCATGCAGGAG
TTGGCGTGCCCAGGAAGCTCTGCCTCTGGGCACAGTGACCTCAGTGGGGTGAGGGGAGCTCTCC
CCATAGCTGGGCTGCGGCCCAACCCCACCCCCTCAGGCTATGCCAGGGGGTGTTGCCAGGGGCA
CCCGGGCATCGCCAGTCTAGCCCACTCCTTCATAAAGCCCTCGCATCCCAGGAGCGAGCAGAGC
CAGAGCAT
Mouse Aldh1L1 promoter-SEQ ID NO: 7
AACTGAGAGTGGAGGGGCACAGAAGAGCCCAAGAGGCTCCTTAGGTTGTGTGGAGGGTACAATA
TGTTTGGGCTGAGCAACCCAGAGCCAGACTTTGTCTGGCTGGTAAGAGACAGAGGTGCCTGCTA
TCACAATCCAAGGGTCTGCTTGAGGCAGAGCCAGTGCAAAGGATGTGGTTAGAGCCAGCCTGGT
GTACTGAAGAGGGGCGAAGAGCTTGAGTAAGGAGTCTCAGCGGTGGTTTGAGAGGCAGGGTGGT
TAATGGAGTAGCTGCAGGGGAGAATCCTTGGGAGGGAGCCTGCAGGACAGAGCTTTGGTCAGGA
AGTGATGGGCATGTCACTGGACCCTGTATTGTCTCTGACTTTTCTCAAGTAGGACAATGACTCT
GCCCAGGGAGGGGGTCTGTGACAAGGTGGAAGGGCCAGAGGAGAACTTCTGAGAAGAAAACCAG
AGGCCGTGAAGAGGTGGGAAGGGCATGGGATTCAGAACCTCAGGCCCACCAGGACACAACCCCA
GGTCCACAGCAGATGGGTGACCTTGCATGTCTCAGTCACCAGCATTGTGCTCCTTGCTTATCAC
GCTTGGGTGAAGGAAATGACCCAAATAGCATAAAGCCTGAAGGCCGGGACTAGGCCAGCTAGGG
CTTGCCCTTCCCTTCCCAGCTGCACTTTCCATAGGTCCCACCTTCAGCAGATTAGACCCGCCTC
CTGCTTCCTGCCTCCTTGCCTCCTCACTCATGGGTCTATGCCCACCTCCAGTCTCGGGACTGAG
GCTCACTGAAGTCCCATCGAGGTCTGGTCTGGTGAATCAGCGGCTGGCTCTGGGCCCTGGGCGA
CCAGTTAGGTTCCGGGCATGCTAGGCAATGAACTCTACCCGGAATTGGGGGTGCGGGGAGGCGG
GGAGGTCTCCAACCCAGCCTTTTGAGGACGTGCCTGTCGCTGCACGGTGCTTTTTATAGACGAT
GGTGGCCCATTTTGCAGAAGGGAAAGCCGGAGCCCTCTGGGGAGCAAGGTCCCCGCAAATGGAC
GGATGACCTGAGCTTGGTTCTGCCAGTCCACTTCCCAAATCCCTCACCCCATTCTAGGGACTAG
GGAAAGATCTCCTGATTGGTCATATCTGGGGGCCTGGCCGGAGGGCCTCCTATGATTGGAGAGA
TCTAGGCTGGGCGGGCCCTAGAGCCCGCCTCTTCTCTGCCTGGAGGAGGAGCACTGACCCTAAC
CCTCTCTGCACAAGACCCGAGCTTGTGCGCCCTTCTGGGAGCTTGCTGCCCCTGTGCTGACTGC
TGACAGCTGACTGACGCTCGCAGCTAGCAGGTACTTCTGGGTTGCTAGCCCAGAGCCCTGGGCC
GGTGACCCTGTTTTCCCTACTTCCCGTCTTTGACCTTGGGTAAGTTTCTTTTTCTTTTGTTTTT
GAGAGAGGCACCCAGATCCTCTCCACTACAGGCAGCCGCTGAACCTTGGATCCTCAGCTCCTGC
CCTGGGAACTACAGTTCCTGCCCTTTTTTTCCCACCTTGAGGGAGGTTTTCCCTGAGTAGCTTC
GACTATCCTGGAACAAGCTTTGTAGACCAGCCTGGGTCTCCGGAGAGTTGGGATTAAAGGCGTG
CACCACCACC
Human NG2 promoter-SEQ ID NO: 8
CTCTGGTTTCAAGACCAATACTCATAACCCCCACATGGACCAGGCACCATCACACCTGAGCACT
GCACTTAGGGTCAAAGACCTGGCCCCACATCTCAGCAGCTATGTAGACTAGCTCCAGTCCCTTA
ATCTCTCTCAGCCTCAGTTTCTTCATCTGCAAAACAGGTCTCAGTTTCGTTGCAAAGTATGAAG
TGCTGGGCTGTTACTGGTCAAAGGGAAGAGCTGGGAAGAGGGTGCAAGGTGGGGTTGGGCTGGA
GATGGGCTGGAGCAGATAGATGGAGGGACCTGAATGGAGGAAGTAAACCAAGGCCCGGTAACAT
TGGGACTGGACAGAGAACACGCAGATCCTCTAGGCACCGGAAGCTAAGTAACATTGCCCTTTCT
CCTCCTGTTTGGGACTAGGCTGATGTTGCTGCCTGGAAGGGAGCCAGCAGAAGGGCCCCAGCCT
GAAGCTGTTAGGTAGAAGCCAAATCCAGGGCCAGATTTCCAGGAGGCAGCCTCGGGAAGTTGAA
ACACCCGGATTCAGGGGTCAGGAGGCCTGGGCTTCTGGCACCAAACGGCCAGGGACCTACTTTC
CACCTGGAGTCTTGTAAGAGCCACTTTCAGCTTGAGCTGCACTTTCGTCCTCCATGAAATGGGG
GAGGGGATGCTCCTCACCCACCTTGCAAGGTTATTTTGAGGCAAATGTCATGGCGGGACTGAGA
ATTCTTCTGCCCTGCGAGGAAATCCAGACATCTCTCCCTTACAGACAGGGAGACTGAGGTGAGG
CCCTTCCAGGCAGAGAAGGTCACTGTTGCAGCCATGGGCAGTGCCCCACAGGACCTCGGGTGGT
GCCTCTGGAGTCTGGAGAAGTTCCTAGGGGACCTCCGAGGCAAAGCAGCCCAAAAGCCGCCTGT
GAGGGTGGCTGGTGTCTGTCCTTCCTCCTAAGGCTGGAGTGTGCCTGTGGAGGGGTCTCCTGAA
CTCCCGCAAAGGCAGAAAGGAGGGAAGTAGGGGCTGGGACAGTTCATGCCTCCTCCCTGAGGGG
GTCTCCCGGGCTCGGCTCTTGGGGCCAGAGTTCAGGGTGTCTGGGCCTCTCTATGACTTTGTTC
TAAGTCTTTAGGGTGGGGCTGGGGTCTGGCCCAGCTGCAAGGGCCCCCTCACCCCTGCCCCAGA
GAGGAACAGCCCCGCACGGGCCCTTTAAGAAGGTTGAGGGTGGGGGCAGGTGGGGGAGTCCAAG
CCTGAAACCCGAGCGGGCGCGCGGGTCTGCGCCTGCCCCGCCCCCGGAGTTAAGTGCGCGGACA
CCCGGAGCCGGCCCGCGCCCAGGAGCAGAGCCGCGCTCGCTCCACTCAGCTCCCAGCTCCCAGG
ACTCCGCTGGCTCCTCGCAAGTCCTGCCGCCCAGCCCGCCGGG
CAG::NeuroD1-IRES-GFP-SEQ ID NO: 9
GATCCGGCCATTAGCCATATTATTCATTGGTTATATAGCATAAATCAATATTGGCTATTGGCCA
TTGCATACGTTGTATCCATATCATAATATGTACATTTATATTGGCTCATGTCCAACATTACCGC
CATGTTGACATTGATTATTGACTAGTTATTAATAGTAATCAATTACGGGGTCATTAGTTCATAG
CCCATATATGGAGTTCCGCGTTACATAACTTACGGTAAATGGCCCGCCTGGCTGACCGCCCAAC
GACCCCCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTGTATCA
TATGCCAAGTACGCCCCCTATTGACGTCAATGACGGTAAATGGCCCGCCTGGCATTATGCCCAG
TACATGACCTTATGGGACTTTCCTACTTGGCAGTACATCTACGTATTAGTCATCGCTATTACCA
TGGTGATGCGGTTTTGGCAGTACATCAATGGGCGTGGATAGCGGTTTGACTCACGGGGATTTCC
AAGTCTCCACCCCATTGACGTCAATGGGAGTTTGTTTTGGCACCAAAATCAACGGGACTTTCCA
AAATGTCGTAACAACTCCGCCCCATTGACGCAAATGGGCGGTAGGCATGTACGGTGGGAGGTCT
ATATAAGCAGAGCTCAATAAAAGAGCCCACAACCCCTCACTCGGGGCGCCAGTCCTCCGATTGA
CTGAGTCGCCCGGGTACCCGTATTCCCAATAAAGCCTCTTGCTGTTTGCATCCGAATCGTGGTC
TCGCTGTTCCTTGGGAGGGTCTCCTCTGAGTGATTGACTACCCACGACGGGGGTCTTTCATTTG
GGGGCTCGTCCGGGATTTGGAGACCCCTGCCCAGGGACCACCGACCCACCACCGGGAGGTAAGC
TGGCCAGCAACTTATCTGTGTCTGTCCGATTGTCTAGTGTCTATGTTTGATGTTATGCGCCTGC
GTCTGTACTAGTTAGCTAACTAGCTCTGTATCTGGCGGACCCGTGGTGGAACTGACGAGTTCTG
AACACCCGGCCGCAACCCTGGGAGACGTCCCAGGGACTTTGGGGGCCGTTTTTGTGGCCCGACC
TGAGGAAGGGAGTCGATGTGGAATCCGACCCCGTCAGGATATGTGGTTCTGGTAGGAGACGAGA
ACCTAAAACAGTTCCCGCCTCCGTCTGAATTTTTGCTTTCGGTTTGGAACCGAAGCCGCGCGTC
TTGTCTGCTGCAGCGCTGCAGCATCGTTCTGTGTTGTCTCTGTCTGACTGTGTTTCTGTATTTG
TCTGAAAATTAGGGCCAGACTGTTACCACTCCCTTAAGTTTGACCTTAGGTCACTGGAAAGATG
TCGAGCGGATCGCTCACAACCAGTCGGTAGATGTCAAGAAGAGACGTTGGGTTACCTTCTGCTC
TGCAGAATGGCCAACCTTTAACGTCGGATGGCCGCGAGACGGCACCTTTAACCGAGACCTCATC
ACCCAGGTTAAGATCAAGGTCTTTTCACCTGGCCCGCATGGACACCCAGACCAGGTCCCCTACA
TCGTGACCTGGGAAGCCTTGGCTTTTGACCCCCCTCCCTGGGTCAAGCCCTTTGTACACCCTAA
GCCTCCGCCTCCTCTTCCTCCATCCGCCCCGTCTCTCCCCCTTGAACCTCCTCGTTCGACCCCG
CCTCGATCCTCCCTTTATCCAGCCCTCACTCCTTCTCTAGGCGCCGGAATTCGATGTCGACATT
GATTATTGACTAGTTATTAATAGTAATCAATTACGGGGTCATTAGTTCATAGCCCATATATGGA
GTTCCGCGTTACATAACTTACGGTAAATGGCCCGCCTGGCTGACCGCCCAACGACCCCCGCCCA
TTGACGTCAATAATGACGTATGTTCCCATAGTAACGCCAATAGGGACTTTCCATTGACGTCAAT
GGGTGGACTATTTACGGTAAACTGCCCACTTGGCAGTACATCAAGTGTATCATATGCCAAGTAC
GCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGGTCGAGGTG
AGCCCCACGTTCTGCTTCACTCTCCCCATCTCCCCCCCCTCCCCACCCCCAATTTTGTATTTAT
TTATTTTTTAATTATTTTGTGCAGCGATGGGGGCGGGGGGGGGGGGGGCGCGCGCCAGGCGGGG
CGGGGCGGGGCGAGGGGCGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAAAGCGAAGC
GCGCGGCGGGCGGGAGTCGCTGCGTTGCCTTCGCCCCGTGCCCCGCTCCGCGCCGCCTCGCGCC
GCCCGCCCCGGCTCTGACTGACCGCGTTACTCCCACAGGTGAGCGGGCGGGACGGCCCTTCTCC
TCCGGGCTGTAATTAGCGCTTGGTTTAATGACGGCTCGTTTCTTTTCTGTGGCTGCGTGAAAGC
CTTAAAGGGCTCCGGGAGGGCCCTTTGTGCGGGGGGGAGCGGCTCGGGGGGTGCGTGCGTGTGT
GTGTGCGTGGGGAGCGCCGCGTGCGGCCCGCGCTGCCCGGCGGCTGTGAGCGCTGCGGGCGCGG
CGCGGGGCTTTGTGCGCTCCGCGTGTGCGCGAGGGGAGCGCGGCCGGGGGCGGTGCCCCGCGGT
GCGGGGGGGCTGCGAGGGGAACAAAGGCTGCGTGCGGGGTGTGTGCGTGGGGGGGTGAGCAGGG
GGTGTGGGCGCGGCGGTCGGGCTGTAACCCCCCCCTGCACCCCCCTCCCCGAGTTGCTGAGCAC
GGCCCGGCTTCGGGTGCGGGGCTCCGTGCGGGGCGTGGCGCGGGGCTCGCCGTGCCGGGCGGGG
GGTGGCGGCAGGTGGGGGTGCCGGGCGGGGCGGGGCCGCCTCGGGCCGGGGAGGGCTCGGGGGA
GGGGCGCGGCGGCCCCGGAGCGCCGGCGGCTGTCGAGGCGCGGCGAGCCGCAGCCATTGCCTTT
TATGGTAATCGTGCGAGAGGGCGCAGGGACTTCCTTTGTCCCAAATCTGGCGGAGCCGAAATCT
GGGAGGCGCCGCCGCACCCCCTCTAGCGGGCGCGGGCGAAGCGGTGCGGCGCCGGCAGGAAGGA
AATGGGCGGGGAGGGCCTTCGTGCGTCGCCGCGCCGCCGTCCCCTTCTCCATCTCCAGCCTCGG
GGCTGCCGCAGGGGGACGGCTGCCTTCGGGGGGGACGGGGCAGGGCGGGGTTCGGCTTCTGGCG
TGTGACCGGCGGCTCTAGAGCCTCTGCTAACCATGTTCATGCCTTCTTCTTTTTCCTACAGCTC
CTGGGCAACGTGCTGGTTGTTGTGCTGTCTCATCATTTTGGCAAAGAATTCGCTAGCGGATCCG
GCCGCCTCGGCCACCGGTCGCCACCATCGCCACCATGACCAAATCATACAGCGAGAGCGGGCTG
ATGGGCGAGCCTCAGCCCCAAGGTCCCCCAAGCTGGACAGATGAGTGTCTCAGTTCTCAGGACG
AGGAACACGAGGCAGACAAGAAAGAGGACGAGCTTGAAGCCATGAATGCAGAGGAGGACTCTCT
GAGAAACGGGGGAGAGGAGGAGGAGGAAGATGAGGATCTAGAGGAAGAGGAGGAAGAAGAAGAG
GAGGAGGAGGATCAAAAGCCCAAGAGACGGGGTCCCAAAAAGAAAAAGATGACCAAGGCGCGCC
TAGAACGTTTTAAATTAAGGCGCATGAAGGCCAACGCCCGCGAGCGGAACCGCATGCACGGGCT
GAACGCGGCGCTGGACAACCTGCGCAAGGTGGTACCTTGCTACTCCAAGACCCAGAAACTGTCT
AAAATAGAGACACTGCGCTTGGCCAAGAACTACATCTGGGCTCTGTCAGAGATCCTGCGCTCAG
GCAAAAGCCCTGATCTGGTCTCCTTCGTACAGACGCTCTGCAAAGGTTTGTCCCAGCCCACTAC
CAATTTGGTCGCCGGCTGCCTGCAGCTCAACCCTCGGACTTTCTTGCCTGAGCAGAACCCGGAC
ATGCCCCCGCATCTGCCAACCGCCAGCGCTTCCTTCCCGGTGCATCCCTACTCCTACCAGTCCC
CTGGACTGCCCAGCCCGCCCTACGGCACCATGGACAGCTCCCACGTCTTCCACGTCAAGCCGCC
GCCACACGCCTACAGCGCAGCTCTGGAGCCCTTCTTTGAAAGCCCCCTAACTGACTGCACCAGC
CCTTCCTTTGACGGACCCCTCAGCCCGCCGCTCAGCATCAATGGCAACTTCTCTTTCAAACACG
AACCATCCGCCGAGTTTGAAAAAAATTATGCCTTTACCATGCACTACCCTGCAGCGACGCTGGC
AGGGCCCCAAAGCCACGGATCAATCTTCTCTTCCGGTGCCGCTGCCCCTCGCTGCGAGATCCCC
ATAGACAACATTATGTCTTTCGATAGCCATTCGCATCATGAGCGAGTCATGAGTGCCCAGCTTA
ATGCCATCTTTCACGATTAGGTTTAAACGCGGCCGCGCCCCTCTCCCTCCCCCCCCCCTAACGT
TACTGGCCGAAGCCGCTTGGAATAAGGCCGGTGTGCGTTTGTCTATATGTTATTTTCCACCATA
TTGCCGTCTTTTGGCAATGTGAGGGCCCGGAAACCTGGCCCTGTCTTCTTGACGAGCATTCCTA
GGGGTCTTTCCCCTCTCGCCAAAGGAATGCAAGGTCTGTTGAATGTCGTGAAGGAAGCAGTTCC
TCTGGAAGCTTCTTGAAGACAAACAACGTCTGTAGCGACCCTTTGCAGGCAGCGGAACCCCCCA
CCTGGCGACAGGTGCCTCTGCGGCCAAAAGCCACGTGTATAAGATACACCTGCAAAGGCGGCAC
AACCCCAGTGCCACGTTGTGAGTTGGATAGTTGTGGAAAGAGTCAAATGGCTCTCCTCAAGCGT
ATTCAACAAGGGGCTGAAGGATGCCCAGAAGGTACCCCATTGTATGGGATCTGATCTGGGGCCT
CGGTGCACATGCTTTACATGTGTTTAGTCGAGGTTAAAAAAACGTCTAGGCCCCCCGAACCACG
GGGACGTGGTTTTCCTTTGAAAAACACGATGATAATATGGCCACAACCATGGTGAGCAAGGGCG
AGGAGCTGTTCACCGGGGTGGTGCCCATCCTGGTCGAGCTGGACGGCGACGTAAACGGCCACAA
GTTCAGCGTGTCCGGCGAGGGCGAGGGCGATGCCACCTACGGCAAGCTGACCCTGAAGTTCATC
TGCACCACCGGCAAGCTGCCCGTGCCCTGGCCCACCCTCGTGACCACCCTGACCTACGGCGTGC
AGTGCTTCAGCCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGACCCGCGCCGAG
GTGAAGTTCGAGGGCGACACCCTGGTGAACCGCATCGAGCTGAAGGGCATCGACTTCAAGGAGG
ACGGCAACATCCTGGGGCACAAGCTGGAGTACAACTACAACAGCCACAACGTCTATATCATGGC
CGACAAGCAGAAGAACGGCATCAAGGTGAACTTCAAGATCCGCCACAACATCGAGGACGGCAGC
GTGCAGCTCGCCGACCACTACCAGCAGAACACCCCCATCGGCGACGGCCCCGTGCTGCTGCCCG
ACAACCACTACCTGAGCACCCAGTCCGCCCTGAGCAAAGACCCCAACGAGAAGCGCGATCACAT
GGTCCTGCTGGAGTTCGTGACCGCCGCCGGGATCACTCTCGGCATGGACGAGCTGTACAAGTAA
GTCGACAATCAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGCTTCCCGTAT
GGCTTTCATTTTCTCCTCCTTGTATAAATCCTGGTTGCTGTCTCTTTATGAGGAGTTGTGGCCC
GTTGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCGGA
ACTCATCGCCGCCTGCCTTGCCCGCTGCTGGACAGGGGCTCGGCTGTTGGGCACTGACAATTCC
GTGGTGTTGTCGGGGAAGCTGACGTCCTTTCCATGGCTGCTCGCCTGTGTTGCCACCTGGATTC
TGCGCGGGACGTCCTTCTGCTACGTCCCTTCGGCCCTCAATCCAGCGGACCTTCCTTCCCGCGG
CCTGCTGCCGGCTCTGCGGCCTCTTCCGCGTCTTCGCCTTCGCCCTCAGACGAGTCGGATCTCC
CTTTGGGCCGCCTCCCCGCCTGGAATTCGAGCTCGAGCTTGTTAACATCGATAAAATAAAAGAT
TTTATTTAGTCTCCAGAAAAAGGGGGGAATGAAAGACCCCACCTGTAGGTTTGGCAAGCTAGCT
TAAGTAACGCCATTTTGCAAGGCATGGAAAAATACATAACTGAGAATAGAGAAGTTCAGATCAA
GGTCAGGAACAGATGGAACAGCTGAATATGGGCCAAACAGGATATCTGTGGTAAGCAGTTCCTG
CCCCGGCTCAGGGCCAAGAACAGATGGAACAGCTGAATATGGGCCAAACAGGATATCTGTGGTA
AGCAGTTCCTGCCCCGGCTCAGGGCCAAGAACAGATGGTCCCCAGATGCGGTCCAGCCCTCAGC
AGTTTCTAGAGAACCATCAGATGTTTCCAGGGTGCCCCAAGGACCTGAAATGACCCTGTGCCTT
ATTTGAACTAACCAATCAGTTCGCTTCTCGCTTCTGTTCGCGCGCTTCTGCTCCCCGAGCTCAA
TAAAAGAGCCCACAACCCCTCACTCGGGGCGCCAGTCCTCCGATTGACTGAGTCGCCCGGGTAC
CCGTGTATCCAATAAACCCTCTTGCAGTTGCATCCGACTTGTGGTCTCGCTGTTCCTTGGGAGG
GTCTCCTCTGAGTGATTGACTACCCGTCAGCGGGGGTCTTTCATTTCCGACTTGTGGTCTCGCT
GCCTTGGGAGGGTCTCCTCTGAGTGATTGACTACCCGTCAGCGGGGGTCTTCACATGCAGCATG
TATCAAAATTAATTTGGTTTTTTTTCTTAAGTATTTACATTAAATGGCCATAGTTGCATTAATG
AATCGGCCAACGCGCGGGGAGAGGCGGTTTGCGTATTGGCGCTCTTCCGCTTCCTCGCTCACTG
ACTCGCTGCGCTCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGCCAGCAAAAGGCC
AGGAACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCGACCGCTGCGC
CTTATCCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAAGTGGT
GGCCTAACTACGGCTACACTAGAAGGACAGTATTTGGTATCTGCGCTCTGCTGAAGCCAGTTAC
CTTCGGAAAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTTGATCTTTT
CTACGGGGTCTGACGCTCAGTGGAACGAAAACTCACGTTAAGGGATTTTGGTCATGAGATTATC
AAAAAGGATCTTCACCTAGATCCTTTTAAATTAAAAATGAAGTTTGCGGCCGGCCGCAAATCAA
TCTAAAGTATATATGAGTAAACTTGGTCTGACAGTTACCAATGCTTAATCAGTGAGGCACCTAT
CTCAGCGATCTGTCTATTTCGTTCATCCATAGTTGCCTGACTCCCCGTCGTGTAGATAACTACG
ATACGGGAGGGCTTACCATCTGGCCCCAGTGCTGCAATGATACCGCGAGACCCACGCTCACCGG
CTCCAGATTTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGTCGTTTGGTA
TGGCTTCATTCAGCTCCGGTTCCCAACGATCAAGGCGAGTTACATGATCCCCCATGTTGTGCAA
AAAAGCGGTTAGCTCCTTCGGTCCTCCGATCGTTGTCAGAAGTAAGTTGGCCGCAGTGTTATCA
CTCATGGTTATGGCAGCACTGCATAATTCTCTTACTGTCATGCCATCCGTAAGATGCTTTTCTG
TGACTGGTGAGTACTCAACCAAGTCATTCTGAGAATAGTGTATGCGGCGACCGAGTTGCTCTTG
CCCGGCGTCAACACGGGATAATACCGCGCCACATAGCAGAACTTTAAAAGTGCTCATCATTGGA
AAACGTTCTTCGGGGCGAAAACTCTCAAGGATCTTACCGCTGTTGAGATCCAGTTCGATGTAAC
CCACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCATA
CTCTTCCTTTTTCAAT

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-13. (canceled)

14. A method of treating a mammal having Alzheimer's disease, wherein said method comprises administering a pharmaceutical composition comprising a pharmaceutically acceptable carrier containing adeno-associated virus particles comprising a nucleic acid encoding a NeuroD1 polypeptide or a biologically active fragment thereof to the brain of said mammal.

15. The method of claim 14, wherein the pharmaceutical composition comprises about 1 μL to about 500 μL of a pharmaceutically acceptable carrier containing adeno-associated virus particles at a concentration of 10¹⁰-10¹⁴ adeno-associated virus particles/mL of carrier.

16. The method of claim 14 or 15, wherein the pharmaceutical composition is injected in the brain of said mammal at a controlled flow rate of about 0.1 μL/minute to about 5 μL/minute.

17. A method for (1) reducing neurofibrillary tangles of hyperphosphorylated tau protein, (2) reducing aggregation of extracellular amyloid plaques, (3) reducing neuroinflammation, (4) reducing interleukin 1β (IL-1β), (5) generating new glutamatergic neurons, (6) increasing survival of GABAergic neurons, (7) generating new non-reactive astrocytes, (8) reducing the number of reactive astrocytes, or (9) improving memory within a mammal having Alzheimer's disease and in need of said (1), (2), (3), (4), (5), (6), (7), (8) or (9), wherein said method comprises administering a composition comprising exogenous nucleic acid encoding a NeuroD1 polypeptide or a biologically active fragment thereof to said mammal, wherein said (1) hyperphosphorylated neurofibrillary tau protein tangles are reduced, (2) aggregation of extracellular amyloid plaques is reduced, (3) neuroinflammation is reduced, (4) interleukin 1β (IL-1β) levels are reduced, (5) new glutamatergic neurons are generated, (6) survival of GABAergic neurons is increased, (7) new non-reactive astrocytes are generated, (8) the number of reactive astrocytes is reduced, or (9) said memory is improved.

18. The claim of 17, wherein said mammal is a human.

19. The method of claim 17, wherein said administering step comprises delivering an expression vector comprising a nucleic acid encoding a NeuroD1 polypeptide.

20. The method of claim 17, wherein said administering step comprises delivering a recombinant viral expression vector comprising a nucleic acid encoding a NeuroD1 polypeptide.

21. The method of claim 17, wherein said administering step comprises delivering a recombinant adeno-associated virus expression vector comprising a nucleic acid encoding a NeuroD1 polypeptide.

22. The method of claim 21, wherein said recombinant adeno-associated virus expression vector is an AAV.PHP.eB expression vector.

23. The method of claim 17, wherein said administering step comprises administering a recombinant expression vector comprising a nucleic acid sequence encoding a NeuroD1 polypeptide, wherein said nucleic acid sequence encoding a NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO:2 or SEQ ID NO:4, or a functional fragment thereof.

24. The method of claim 17, wherein said administering step comprises a stereotactic intracranial injection.

25. The method of claim 24, wherein said administering step comprises two or more stereotactic intracranial injections.

26. The method of claim 17, wherein said administering step comprises an extracranial injection.

27. The method of claim 26, wherein said administering step comprises two or more extracranial injections.

28. The method of claim 17, wherein said administering step comprises a retro-orbital injection.


SEQUENCE LISTING (9 sequences)

SEQ ID NO: 1 - DNA, 1071 bp, Homo sapiens
atgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattag

SEQ ID NO: 2 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 3 - DNA, 1074 bp, Mus musculus
atgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactgtctaaaatagagacactgcgcttggccaagaactacatctgggctctgtcagagatcctgcgctcaggcaaaagccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccagtcccctggactgcccagcccgccctacggcaccatggacagctcccacgtcttccacgtcaagccgccgccacacgcctacagcgcagctctggagcccttctttgaaagccccctaactgactgcaccagcccttcctttgacggacccctcagcccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattag

SEQ ID NO: 4 - protein, 357 aa, Mus musculus
MTKSYSESGLMGEPQPQGPPSWTDECLSSQDEEHEADKKEDELEAMNAEEDSLRNGGEEEEEDEDLEEEEEEEEEEEDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNPDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGPQSHGSIFSSGAAAPRCEIPIDNIMSFDSHSHHERVMSAQLNAIFHD

SEQ ID NO: 5 - DNA, 1053 bp, Mus musculus
gcagtgtggagacacacccactttccccaagggctcctgctcccccaagtgatccccttatcctccgtgctaagatgacaccgaggttgcagtccttacctttgaaagcagccacaagggcgtgggggtgcacacctttaatcccagcactcgggaggcagaggcaggcagatttctgagttcgagaccagcctggtctacaaagtgaattccaggacagccagggctatacagagaaaccctgtcttgaaaaaaaaagagaaagaaaaaagaaaaaaaaaaatgaaagcagccacatctaaggactacgtggcacaggagagggtgagtccctgagagttcagctgctgccctgtctgttcctgtaaatggcagtggggtcatgggaaagtgaaggggctcaaggtattggacacttccaggataatcttttggacgcctcaccctgtgccaggaccaaggctgagcttggcaggctcagaacagggtgtcctgttcttccctgtctaaaacattcactctcagcttgctcacccttccccagacaaggaagctgcacagggtctggtgttcagatggctttggcttacagcaggtgtgggtgtggggtaggaggcagggggtaggggtgggggaagcctgtactatactcactatcctgtttctgaccctctaggactcctacagggttatgggagtggacaggcagtccagatctgagctgctgacccacaagcagtgccctgtgcctgccagaatccaaagccctgggaatgtccctctggtccccctctgtcccctgcagcccttcctgttgctcaaccttgcacagttccgacctgggggagagagggacagaaatcttgccaagtatttcaacagaatgtactggcaattacttcatggcttcctggacttggtaaaggatggactaccccgcccaacaggggggctggcagccaggtaggcccataaaaagcccgctggggagtcctcctcactctctgctcttcctcctccagcacacatcagacctagtagctgtggaaacca

SEQ ID NO: 6 - DNA, 1672 bp, Homo sapiens
gtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagtagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgattcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcat

SEQ ID NO: 7 - DNA, 1610 bp, Mus musculus
aactgagagtggaggggcacagaagagcccaagaggctccttaggttgtgtggagggtacaatatgtttgggctgagcaacccagagccagactttgtctggctggtaagagacagaggtgcctgctatcacaatccaagggtctgcttgaggcagagccagtgcaaaggatgtggttagagccagcctggtgtactgaagaggggcgaagagcttgagtaaggagtctcagcggtggtttgagaggcagggtggttaatggagtagctgcaggggagaatccttgggagggagcctgcaggacagagctttggtcaggaagtgatgggcatgtcactggaccctgtattgtctctgacttttctcaagtaggacaatgactctgcccagggagggggtctgtgacaaggtggaagggccagaggagaacttctgagaagaaaaccagaggccgtgaagaggtgggaagggcatgggattcagaacctcaggcccaccaggacacaaccccaggtccacagcagatgggtgaccttgcatgtctcagtcaccagcattgtgctccttgcttatcacgcttgggtgaaggaaatgacccaaatagcataaagcctgaaggccgggactaggccagctagggcttgcccttcccttcccagctgcactttccataggtcccaccttcagcagattagacccgcctcctgcttcctgcctccttgcctcctcactcatgggtctatgcccacctccagtctcgggactgaggctcactgaagtcccatcgaggtctggtctggtgaatcagcggctggctctgggccctgggcgaccagttaggttccgggcatgctaggcaatgaactctacccggaattgggggtgcggggaggcggggaggtctccaacccagccttttgaggacgtgcctgtcgctgcacggtgctttttatagacgatggtggcccattttgcagaagggaaagccggagccctctggggagcaaggtccccgcaaatggacggatgacctgagcttggttctgccagtccacttcccaaatccctcaccccattctagggactagggaaagatctcctgattggtcatatctgggggcctggccggagggcctcctatgattggagagatctaggctgggcgggccctagagcccgcctcttctctgcctggaggaggagcactgaccctaaccctctctgcacaagacccgagcttgtgcgcccttctgggagcttgctgcccctgtgctgactgctgacagctgactgacgctcgcagctagcaggtacttctgggttgctagcccagagccctgggccggtgaccctgttttccctacttcccgtctttgaccttgggtaagtttctttttcttttgtttttgagagaggcacccagatcctctccactacaggcagccgctgaaccttggatcctcagctcctgccctgggaactacagttcctgccctttttttcccaccttgagggaggttttccctgagtagcttcgactatcctggaacaagctttgtagaccagcctgggtctccggagagttgggattaaaggcgtgcaccaccacc

SEQ ID NO: 8 - DNA, 1387 bp, Homo sapiens
ctctggtttcaagaccaatactcataacccccacatggaccaggcaccatcacacctgagcactgcacttagggtcaaagacctggccccacatctcagcagctatgtagactagctccagtcccttaatctctctcagcctcagtttcttcatctgcaaaacaggtctcagtttcgttgcaaagtatgaagtgctgggctgttactggtcaaagggaagagctgggaagagggtgcaaggtggggttgggctggagatgggctggagcagatagatggagggacctgaatggaggaagtaaaccaaggcccggtaacattgggactggacagagaacacgcagatcctctaggcaccggaagctaagtaacattgccctttctcctcctgtttgggactaggctgatgttgctgcctggaagggagccagcagaagggccccagcctgaagctgttaggtagaagccaaatccagggccagatttccaggaggcagcctcgggaagttgaaacacccggattcaggggtcaggaggcctgggcttctggcaccaaacggccagggacctactttccacctggagtcttgtaagagccactttcagcttgagctgcactttcgtcctccatgaaatgggggaggggatgctcctcacccaccttgcaaggttattttgaggcaaatgtcatggcgggactgagaattcttctgccctgcgaggaaatccagacatctctcccttacagacagggagactgaggtgaggcccttccaggcagagaaggtcactgttgcagccatgggcagtgccccacaggacctcgggtggtgcctctggagtctggagaagttcctaggggacctccgaggcaaagcagcccaaaagccgcctgtgagggtggctggtgtctgtccttcctcctaaggctggagtgtgcctgtggaggggtctcctgaactcccgcaaaggcagaaaggagggaagtaggggctgggacagttcatgcctcctccctgagggggtctcccgggctcggctcttggggccagagttcagggtgtctgggcctctctatgactttgttctaagtctttagggtggggctggggtctggcccagctgcaagggccccctcacccctgccccagagaggaacagccccgcacgggccctttaagaaggttgagggtgggggcaggtgggggagtccaagcctgaaacccgagcgggcgcgcgggtctgcgcctgccccgcccccggagttaagtgcgcggacacccggagccggcccgcgcccaggagcagagccgcgctcgctccactcagctcccagctcccaggactccgctggctcctcgcaagtcctgccgcccagcccgccggg

SEQ ID NO: 9 - DNA, 9232 bp, Artificial
  vector construct
gatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcaataaaagagcccacaacccctcactcggggcgccagtcctccgattgactgagtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcgatgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcgctagcggatccggccgcctcggccaccggtcgccaccatcgccaccatgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactgtctaaaatagagacactgcgcttggccaagaactacatctgggctctgtcagagatcctgcgctcaggcaaaagccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccagtcccctggactgcccagcccgccctacggcaccatggacagctcccacgtcttccacgtcaagccgccgccacacgcctacagcgcagctctggagcccttctttgaaagccccctaactgactgcaccagcccttcctttgacggacccctcagcccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattaggtttaaacgcggccgcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcgagcttgttaacatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttccgacttgtggtctcgctgccttgggagggtctcctctgagtgattgactacccgtcagcgggggtcttcacatgcagcatgtatcaaaattaatttggttttttttcttaagtatttacattaaatggccatagttgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagtttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat